SEQUENCE LISTING (91 sequences)

SEQ ID NO: 1 - protein, 111 aa, Artificial Sequence
  synthetic polypeptide
DIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIK

SEQ ID NO: 2 - protein, 111 aa, Artificial Sequence
  synthetic polypeptide
DIVLTQSPASLAVSLGQRATISCRASKSVSTSSYSYMHWYQQKPGQPPKLLIKYASYLESGVPARFSGSGSGTDFHLNIHPVEEEDAATYYCQHSREFPWTFGGGTKLELK

SEQ ID NO: 3 - protein, 111 aa, Artificial Sequence
  synthetic polypeptide
DIQMTQSPSSLSASVGDRVTITCRASKSVSTSSYSYLAWYQQKPEKAPKLLIKYASYLQSGVPSRFSGSGSGTDFTLTISSLQPEDFATYYCQHSREFPWTFGAGTKLELK

SEQ ID NO: 4 - protein, 111 aa, Artificial Sequence
  synthetic polypeptide
DIQMTQSPSSLSASVGDRVTISCRASKSVSTSSYSYMHWYQQKPEKAPKLLIKYASYLQSGVPSRFSGSGSGTDFTLTISSLQPEDVATYYCQHSREFPWTFGAGTKLELK

SEQ ID NO: 5 - protein, 111 aa, Artificial Sequence
  synthetic polypeptide
DIVLTQSPASLAVSPGQRATITCRASKSVSTSSYSYMHWYQQKPGQPPKLLIYYASYLESGVPARFSGSGSGTDFTLTINPVEANDTANYYCQHSREFPWTFGQGTKVEIK

SEQ ID NO: 6 - protein, 116 aa, Artificial Sequence
  synthetic polypeptide
EVQLVESGGGLVKPGGSRKLSCAASGFTFSDYGMHWVRQAPEKGLEWVAYISSGSSTIYYADTVKGRFTISRDNAKNTLFLQMTSLRSEDTAMYYCARRGLLLDYWGQGTTLTVSS

SEQ ID NO: 7 - protein, 116 aa, Artificial Sequence
  synthetic polypeptide
EVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSS

SEQ ID NO: 8 - protein, 5 aa, Artificial Sequence
  synthetic polypeptide
DYGMH

SEQ ID NO: 9 - protein, 116 aa, Artificial Sequence
  synthetic polypeptide
EVQLVQSGGGLIQPGGSLRLSCAASGFTFSNYGMHWVRQAPGKGLEWVSYISSGSSTIYYADSVKGRFTISRDNSKNTLYLQMNSLRAEDTAVYYCARRGLLLDYWGQGTTVTVSS

SEQ ID NO: 10 - protein, 17 aa, Artificial Sequence
  synthetic polypeptide
YISSGSSTIYYADTVKG

SEQ ID NO: 11 - protein, 116 aa, Artificial Sequence
  synthetic polypeptide
EVQLVESGGGLIQPGGSLRLSCAASGFTFSNYGMHWVRQAPGKGLEWVSYISSGSSTIYYADSVKGRFTISRDNSKNTLYLQMTSLRAEDTAVYYCARRGLLLDYWGQGTTLTVSS

SEQ ID NO: 12 - protein, 112 aa, Artificial Sequence
  synthetic polypeptide
DIVLTQSPASLAAVSLGERATISYRASKSVSTSGYSYMHWNQQKPGQAPRLLIYLVSNLESGVPARFSGSGSGTDFTLNIHPVEEEDAATYYCQHIRELDTFFGGGTKLEIK

SEQ ID NO: 13 - protein, 15 aa, Artificial Sequence
  synthetic polypeptide
RASKSVSTSGYSYMH

SEQ ID NO: 14 - protein, 7 aa, Artificial Sequence
  synthetic polypeptide
LVSNLES

SEQ ID NO: 15 - protein, 9 aa, Artificial Sequence
  synthetic polypeptide
QHIRELDTF

SEQ ID NO: 16 - protein, 120 aa, Artificial Sequence
  synthetic polypeptide
QLKLVESGGGLVKPGGSLKLSCAASGFTFSSYTMSWVRQTPAKRLEWVATISSGGGSTYYPDSVKGRFTISRDNARNTLYLQMSSLRSEDTAMYYCARRAYSKRGAMDYWGQGTSVTVSS

SEQ ID NO: 17 - protein, 5 aa, Artificial Sequence
  synthetic polypeptide
SYTMS

SEQ ID NO: 18 - protein, 17 aa, Artificial Sequence
  synthetic polypeptide
TISSGGGSTYYPDSVKG

SEQ ID NO: 19 - protein, 11 aa, Artificial Sequence
  synthetic polypeptide
RAYSKRGAMDY

SEQ ID NO: 20 - protein, 7 aa, Artificial Sequence
  synthetic polypeptide
GQSSRSS

SEQ ID NO: 21 - protein, 18 aa, Artificial Sequence
  synthetic polypeptide
GQSSRSSSGGGSSGGGGS

SEQ ID NO: 22 - protein, 4 aa, Artificial Sequence
  synthetic polypeptide
GSGS

SEQ ID NO: 23 - protein, 4 aa, Artificial Sequence
  synthetic polypeptide
GGGS

SEQ ID NO: 24 - protein, 10 aa, Artificial Sequence
  synthetic polypeptide
GGGGSGGGGS

SEQ ID NO: 25 - protein, 20 aa, Artificial Sequence
  synthetic polypeptide
GGGGSGGGGSGGGGSGGGGS

SEQ ID NO: 26 - protein, 15 aa, Artificial Sequence
  synthetic polypeptide
GGGGSGGGGSGGGGS

SEQ ID NO: 27 - protein, 274 aa, Artificial Sequence
  synthetic polypeptide
AGIHDIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRADAAPGGGGSGGGGSGGGGSEVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSSLEQKLISEEDLNSAVDHHHHHH

SEQ ID NO: 28 - protein, 541 aa, Artificial Sequence
  synthetic polypeptide
AGIHDIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRADAAPGGGGSGGGGSGGGGSEVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSSASTKGPSVFPLAPLESSGSDIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRADAAPGGGGSGGGGSGGGGSEVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSSLEQKLISEEDLNSAVDHHHHHH

SEQ ID NO: 29 - protein, 808 aa, Artificial Sequence
  synthetic polypeptide
AGIHDIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRADAAPGGGGSGGGGSGGGGSEVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSSASTKGPSVFPLAPLESSGSDIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRADAAPGGGGSGGGGSGGGGSEVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSSASTKGPSVFPLAPLESSGSDIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRADAAPGGGGSGGGGSGGGGSEVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSSLEQKLISEEDLNSAVDHHHHHH

SEQ ID NO: 30 - protein, 5 aa, Artificial Sequence
  synthetic polypeptide
NYGMH

SEQ ID NO: 31 - protein, 17 aa, Artificial Sequence
  synthetic polypeptide
YISSSSSTIYYADSVKG

SEQ ID NO: 32 - protein, 17 aa, Artificial Sequence
  synthetic polypeptide
YISSGSSTIYYADSVKG

SEQ ID NO: 33 - protein, 7 aa, Artificial Sequence
  synthetic polypeptide
RGLLLDY

SEQ ID NO: 34 - protein, 15 aa, Artificial Sequence
  synthetic polypeptide
RASKSVSTSSYSYMH

SEQ ID NO: 35 - protein, 15 aa, Artificial Sequence
  synthetic polypeptide
RASKTVSTSSYSYMH

SEQ ID NO: 36 - protein, 7 aa, Artificial Sequence
  synthetic polypeptide
YASYLES

SEQ ID NO: 37 - protein, 9 aa, Artificial Sequence
  synthetic polypeptide
QHSREFPWT

SEQ ID NO: 38 - protein, 8 aa, Artificial Sequence
  synthetic polypeptide
GFTFSNYG

SEQ ID NO: 39 - protein, 8 aa, Artificial Sequence
  synthetic polypeptide
ISSSSSTI

SEQ ID NO: 40 - protein, 8 aa, Artificial Sequence
  synthetic polypeptide
ISSGSSTI

SEQ ID NO: 41 - protein, 9 aa, Artificial Sequence
  synthetic polypeptide
ARRGLLLDY

SEQ ID NO: 42 - protein, 10 aa, Artificial Sequence
  synthetic polypeptide
KSVSTSSYSY

SEQ ID NO: 43 - protein, 10 aa, Artificial Sequence
  synthetic polypeptide
KTVSTSSYSY

SEQ ID NO: 44 - protein, 3 aa, Artificial Sequence
  synthetic polypeptide
YAS

SEQ ID NO: 45 - protein, 116 aa, Artificial Sequence
  synthetic polypeptide
EVQLQESGGGVVQPGGSLRLSCAASGFTFSNYGMHWIRQAPGKGLEWVSYISSGSSTIYYADSVKGRFTISRDNSKNTLYLQMNSLRSEDTAVYYCARRGLLLDYWGQGTLVTVSS

SEQ ID NO: 46 - protein, 116 aa, Artificial Sequence
  synthetic polypeptide
EVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSS

SEQ ID NO: 47 - protein, 116 aa, Artificial Sequence
  synthetic polypeptide
EVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 48 - protein, 116 aa, Artificial Sequence
  synthetic polypeptide
EVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 49 - protein, 116 aa, Artificial Sequence
  synthetic polypeptide
EVQLVESGGGLVQPGGSLRLSCSASGFTFSNYGMHWVRQAPGKGLEYVSYISSGSSTIYYADTVKGRFTISRDNSKNTLYLQMSSLRAEDTAVYYCVKRGLLLDYWGQGTLVTVSS

SEQ ID NO: 50 - protein, 116 aa, Artificial Sequence
  synthetic polypeptide
EVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSS

SEQ ID NO: 51 - protein, 116 aa, Artificial Sequence
  synthetic polypeptide
EVQLVESGGGVVQPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 52 - protein, 116 aa, Artificial Sequence
  synthetic polypeptide
EVQLVESGGGDVKPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 53 - protein, 111 aa, Artificial Sequence
  synthetic polypeptide
DIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIK

SEQ ID NO: 54 - protein, 111 aa, Artificial Sequence
  synthetic polypeptide
DIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIK

SEQ ID NO: 55 - protein, 111 aa, Artificial Sequence
  synthetic polypeptide
DIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIK

SEQ ID NO: 56 - protein, 111 aa, Artificial Sequence
  synthetic polypeptide
DIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIK

SEQ ID NO: 57 - protein, 111 aa, Artificial Sequence
  synthetic polypeptide
DIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIK

SEQ ID NO: 58 - protein, 111 aa, Artificial Sequence
  synthetic polypeptide
DIQMTQSPSSLSASLGDRATITCRASKTVSTSSYSYMHWYQQKPGQAPKLLIKYASYLESGVPSRFSGSGSGTDFTLTISSLQPEDFATYYCQHSREFPWTFGQGTKVEIK

SEQ ID NO: 59 - protein, 21 aa, Artificial Sequence
  synthetic polypeptide
RADAAPGGGGSGGGGSGGGGS

SEQ ID NO: 60 - protein, 19 aa, Artificial Sequence
  synthetic polypeptide
ASTKGPSVFPLAPLESSGS

SEQ ID NO: 61 - protein, 515 aa, Artificial Sequence
  synthetic polypeptide
DIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSS

SEQ ID NO: 62 - protein, 515 aa, Artificial Sequence
  synthetic polypeptide
DIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 63 - protein, 515 aa, Artificial Sequence
  synthetic polypeptide
DIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 64 - protein, 515 aa, Artificial Sequence
  synthetic polypeptide
DIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSS

SEQ ID NO: 65 - protein, 515 aa, Artificial Sequence
  synthetic polypeptide
DIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 66 - protein, 515 aa, Artificial Sequence
  synthetic polypeptide
DIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 67 - protein, 515 aa, Artificial Sequence
  synthetic polypeptide
DIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSSASTKGPSVFPLAPLESSGSDIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSS

SEQ ID NO: 68 - protein, 515 aa, Artificial Sequence
  synthetic polypeptide
DIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 69 - protein, 515 aa, Artificial Sequence
  synthetic polypeptide
DIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 70 - protein, 515 aa, Artificial Sequence
  synthetic polypeptide
DIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSSASTKGPSVFPLAPLESSGSDIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSS

SEQ ID NO: 71 - protein, 515 aa, Artificial Sequence
  synthetic polypeptide
DIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGVVQPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGVVQPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 72 - protein, 515 aa, Artificial Sequence
  synthetic polypeptide
DIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGDVKPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGDVKPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 73 - protein, 515 aa, Artificial Sequence
  synthetic polypeptide
DIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSSASTKGPSVFPLAPLESSGSDIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSS

SEQ ID NO: 74 - protein, 515 aa, Artificial Sequence
  synthetic polypeptide
DIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSSASTKGPSVFPLAPLESSGSDIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 75 - protein, 515 aa, Artificial Sequence
  synthetic polypeptide
DIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSSASTKGPSVFPLAPLESSGSDIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 76 - protein, 515 aa, Artificial Sequence
  synthetic polypeptide
DIQMTQSPSSLSASLGDRATITCRASKTVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSSASTKGPSVFPLAPLESSGSDIQMTQSPSSLSASLGDRATITCRASKTVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSS

SEQ ID NO: 77 - protein, 446 aa, Artificial Sequence
  synthetic polypeptide
EVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 98 aa, Artificial Sequence
  synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 79 - protein, 12 aa, Artificial Sequence
  synthetic polypeptide
EPKSCDKTHTCP

SEQ ID NO: 80 - protein, 113 aa, Artificial Sequence
  synthetic polypeptide
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 81 - protein, 107 aa, Artificial Sequence
  synthetic polypeptide
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 446 aa, Artificial Sequence
  synthetic polypeptide
EVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYDSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 113 aa, Artificial Sequence
  synthetic polypeptide
PCPAPELLGGPSVFLFPPKPKDTLMISRTPEVTCVVVDVSHEDPEVKFNWYVDGVEVHNAKTKPREEQYDSTYRVVSVLTVLHQDWLNGKEYKCKVSNKALPAPIEKTISKAK

SEQ ID NO: 84 - protein, 446 aa, Artificial Sequence
  synthetic polypeptide
EVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYDSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 113 aa, Artificial Sequence
  synthetic polypeptide
PCPAPEAAGGPSVFLFPPKPKDTLMISRTPEVTCVVVDVSHEDPEVKFNWYVDGVEVHNAKTKPREEQYDSTYRVVSVLTVLHQDWLNGKEYKCKVSNKALPAPIEKTISKAK

SEQ ID NO: 86 - protein, 113 aa, Artificial Sequence
  synthetic polypeptide
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 87 - protein, 218 aa, Artificial Sequence
  synthetic polypeptide
DIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 88 - protein, 242 aa, Artificial Sequence
  synthetic polypeptide
DIVLTQSPASLAVSPGQRATITCRASKSVSTSSYSYMHWYQQKPGQPPKLLIYYASYLESGVPARFSGSGSGTDFTLTINPVEANDTANYYCQHSREFPWTFGQGTKVEIKGGGGSGGGGSGGGGSEVQLVESGGGLVQPGGSLRLSCSASGFTFSNYGMHWVRQAPGKGLEYVSYISSGSSTIYYADTVKGRFTISRDNSKNTLYLQMSSLRAEDTAVYYCVKRGLLLDYWGQGTLVTVSS

SEQ ID NO: 89 - protein, 8 aa, Artificial Sequence
  synthetic polypeptide
GFTFSDYG

SEQ ID NO: 90 - protein, 7 aa, Artificial Sequence
  synthetic polypeptide
YASYLQS

SEQ ID NO: 91 - protein, 15 aa, Artificial Sequence
  synthetic polypeptide
RASKSVSTSSYSYLA

(19) United States
(12) Patent Application Publication
(10) Pub. No.: US 2024/0166770 A1
(43) Pub. Date: May 23, 2024

(54) COMPOSITIONS AND METHODS FOR INHIBITION OF CELL-PENETRATING ANTIBODIES

(71) Applicant: Yale University, New Haven, CT (US)

(72) Inventors: James Hansen, Guilford, CT (US); Jiangbing Zhou, Cheshire, CT (US)

(21) Appl. No.: 17/769,642

(22) PCT Filed: Oct. 19, 2020
(86) PCT No.: PCT/US2020/056285

§ 371 Date: Apr. 15, 2022

Related U.S. Application Data
(60) Provisional application No. 62/923,015, filed on Oct. 18, 2019.

(51) Int. Cl. C07K 16/44; A61K 31/519; A61K 39/00; A61P 35/00; C12N 15/113

(52) U.S. Cl. CPC C07K 16/44; A61K 31/519; A61P 35/00; C12N 15/113; A61K 2039/505; C07K 2317/24; C07K 2317/622; C12N 2310/14

(57) ABSTRACT

Compositions and methods of treating autoimmune diseases by administering a subject in need thereof an effective amount of an inhibitor of a nucleoside transporter are provided. Typically, the autoimmune disease has one or more symptoms or pathologies dependent on or otherwise caused by cell penetrating antibodies that transduced into or through cells at in-part by the nucleoside transporter. In specific embodiments, the autoimmune disease is a form of lupus, for example central nervous system lupus. In preferred embodiments, the inhibitor is dipyridamole or a pharmaceutically active analogue thereof. Compositions, formulations, and dosage forms including an effective amount of dipyridamole or analogue to reduce cellular transduction or transcellular transport of the cell penetrating antibody are also provided. The compositions can be employed in the disclosed methods. Exemplary dosages ranges and dosage regimens are also provided.

### CROSS-REFERENCE TO RELATED APPLICATION

This application claims the benefit of and priority to U.S. Ser. No. 62/923,015 filed Oct. 18, 2019 and which is incorporated by referenced herein in its entirety.

### REFERENCE TO THE SEQUENCE LISTING

The Sequence Listing submitted as a text file named “YU_7810_PCT_ST25” created on Oct. 15, 2020, and having a size of 143,536 bytes is hereby incorporated by reference pursuant to 37 C.F.R. § 1.52(e)(5).

### FIELD OF THE INVENTION

This invention is generally related to compositions and methods of use thereof for reducing cell penetration and/or transcellular transport of cell penetrating antibodies and the treatment of diseases and disorders associated thereof.

### BACKGROUND OF THE INVENTION

Aberrant production of autoantibodies reactive against self-antigens results in inflammation and tissue damage that is characteristic of autoimmune diseases such as systemic lupus erythematosus (SLE), scleroderma, Sjogren's syndrome, Hashimoto's thyroiditis, multiple sclerosis, and many others. While most antibodies are targeted to extracellular antigens such as cell surface receptors or circulating factors, a select subset of autoantibodies has the unusual ability to penetrate live cells where they can target intracellular antigens. For example, some antinuclear antibodies (ANAs) penetrate live cells and localize to nuclei, and cause functional perturbations in autoimmune disease (Yanase and Madaio, in Autoimmune Reactions, S. Paul, Ed. (Humana Press, Totowa, NJ, 1999), pp. 293-304; Rhodes and Isenberg, Trends Immunol 38, 916-926 (2017); Ying-Chyi et al., Eur J Immunol 38, 3178-3190 (2008)). Anti-dsDNA antibodies are highly specific for systemic lupus erythematosus (SLE), and elevated titers are detected in ˜70% of SLE patients, compared to 0.5% in healthy individuals or those presenting with other autoimmune disorders (e.g. rheumatoid arthritis) (Rahman and Isenberg, N Engl J Med 358, 929-939 (2008); Isenberg et al., Arthritis Rheum 28, 999-1007 (1985)). The specific contributions of anti-dsDNA antibodies to lupus pathophysiology are unknown, but these antibodies are included in the ACR (American College of Rheumatology) and SLICC (Systemic Lupus International Collaborating Clinics) SLE classification criteria, and are associated with dermatological and renal manifestations of lupus (Yu et al., J Autoimmun 48-49, 10-3 (2014)).

The remains a need for compositions and methods of reducing cellular penetration and/or transcellular transport of autoantibodies.

Thus, it is an object of the invention to identify mechanisms leading to cellular penetration and/or transcellular transport of autoantibodies, and compositions and methods for reducing the same.

### SUMMARY OF THE INVENTION

A modified version of the 3E10 lupus autoantibody (h3E10 di-scFv (SEQ ID NO:70)) crosses the BBB and suppresses the growth of brain tumors. The mechanism by which this modified antibody crosses the BBB has been unclear. The experiments described below show that the nucleoside transporter ENT2 is expressed in human brain endothelial cells, which contribute to the blood-brain barrier (BBB), and regulate nucleoside concentrations in the central nervous system (CNS). h3E10 di-scFv can cross a Transwell filter model of the human BBB. The drug dipyridamole, which inhibits nucleoside transporters including ENT2, inhibits transport of h3E10 di-scFv across the Transwell filter model of the BBB, and inhibits localization of h3E10 di-scFv into brain tumors in vivo.

These results indicate that ENT2 facilitates cellular penetration of h3E10 di-scFv and transcellular transport across the BBB and into the brain to impact brain tumors. Thus, strategies for modulating the use of cell penetrating antibodies in cancer therapy, and, for example, reducing infiltration of the cell penetrating antibodies in the brain are provided. For example, methods of reducing transcellular transport of an antibody into cells or into or through a tissue in a subject can include administering the subject an effective amount of an inhibitor of a nucleoside transporter to reduce transduction into cells and/or transcellular transport of the antibody into or through the tissue. In some embodiments, the cells are BBB cells, for example, human endothelial cells. In some embodiments, the tissue is brain tissue.

In some embodiments, the subject is administered the antibody in an effective amount to inhibit DNA repair or directly damage DNA separately or together with the inhibitor of the nucleoside transporter.

In some embodiments, the subject has cancer, and optionally the antibody is a treatment for the cancer. The antibody can be, for example, 3E10, 5C6, fragments and fusions of 3E10 and 5C6, and variants and humanized forms of 3E10, 5C6, and fragments and fusions of 3E10 and 5C6. In some embodiments, the antibody a humanized 3E10 di-scFv, for example, SEQ ID NO:70, or variant thereof with 90% sequence identity thereto.

Furthermore, 3E10 is a lupus anti-DNA autoantibody. About 40% of lupus patients develop the neurologic syndrome of “CNS lupus.” It has been unclear how autoantibodies are accessing the brain in lupus to contribute to CNS lupus. The experiments described below support the conclusion that nucleoside transporters such as ENT2 in the BBB and other tissues where they are expressed play a role in cell penetration and/or transcellular transport of autoantibodies. Thus, methods and compositions for treating autoimmune diseases are also provided. For example, methods of treating an autoimmune disease can include administering to a subject in need thereof an effective amount of an inhibitor of a nucleoside transporter to reduce cellular transduction, or preferably transcellular transport of one or more cell penetrating antibodies into or through cells or a tissue of the subject, for example, the brain. In some embodiments, the inhibitor reduces transport of the antibody or antibodies across the blood-brain barrier (BBB).

Typically, the autoimmune disease has one or more symptoms or pathology dependent on or otherwise caused by one or more cell penetrating antibodies, which may be, but are not necessarily, nuclear penetrating antibodies. The antibodies typically bind to nucleic acids, nucleotides, nucleotides, or a combination thereof. Typically, the antibodies are internalized and/or transcellularly transported at least in part by the nucleotide transporter.

Exemplary autoimmune diseases include, but are not limited to, systemic lupus erythematosus (lupus or SLE), central nervous system (CNS) lupus, systemic sclerosis (scleroderma), Graves' disease, myasthenia gravis, autoimmune hemolytic anemia, and pemphigus vulgaris, and additionally may contribute to the severity of disease in other autoimmune diseases such as rheumatoid arthritis, autoimmune thrombocytopenia, autoimmune atrophic gastritis of pernicious anemia, myasthenia gravis, Goodpasture's syndrome, diabetes mellitus, multiple sclerosis, Hashimoto's thyroiditis, Crohn's disease, and Sjögren's syndrome, primary biliary cirrhosis, primary sclerosing cholangitis, psoriasis, psoriatic arthritis, POEMS syndrome, dermatomyositis, inclusion body myositis, inflammatory myopathies, vasculitis syndromes including but not limited to Churg-Strauss Syndrome, Wegener granulomatosis, Behcet's disease, Buerger's disease, Kawasaki disease, Takayasu's arteritis, Henoch-Schonlein purpura, Giant cell arteritis, polyarteritis nodosa, and combinations thereof.

In an embodiment, the subject has lupus or SLE. In a particularly preferred embodiments, the subject has CNS lupus.

Methods of treating lupus by administering to a subject in need thereof effective amount of an inhibitor of a nucleoside transporter to reduce one or more symptoms of the lupus, for example CNS lupus, are also provided.

Inhibitors of nucleoside transporters are also provided. The inhibitor of the nucleoside transporter can inhibit expression or activity of one or more of ENT1, ENT2, ENT3, or ENT4, optionally at least or only ENT2. The inhibitor can be, for example, a purine nucleoside analogue, a pyrimidopyrimidine or pteridine derivative, or a flazine calcium channel blocker. In preferred embodiments, the inhibitor is dipyridamole or a tautomer, geometrical isomer, optically active form, enantiomeric mixture thereof, pharmaceutically acceptable salt or pharmaceutically active analogue thereof. Exemplary analogues include the compounds of formula I and Tables 1-5. Other inhibitors include peptides, binding proteins such as inhibitory antibodies and fragments thereof, and oligonucleotide inhibitor such as antisense RNA or DNA, siRNA or siDNA, miRNA, miRNA mimic, shRNA or DNA and chimeric antisense DNA or RNA.

The inhibitor can be administered to a subject in need thereof by a parenteral, enteral, transdermal, or transmucosal route of administration, and may be used in combination with other active agents.

Compositions including an effective amount the disclosed inhibitors to reduce one or more symptoms of an autoimmune disease, particularly CNS lupus, in a subject in need thereof are also provided.

### BRIEF DESCRIPTION OF THE DRAWINGS

FIGS. 1A-1F are dot plots of weeks 1-6, respectively, showing absolute radiance efficiencies of brain metastases tracked by IVIS in mice treated with tail vein injection of control (PBS) or h3E10 di-scFv (20 mg/kg) 3×/week for 4 weeks.

FIG. 2 is a plot showing % survival of mice treated with tail vein injection of control (PBS) or h3E10 di-scFv (20 mg/kg) 3×/week for 4 weeks.

FIG. 3 is a plot showing body weight of mice treated with tail vein injection of control (PBS) or h3E10 di-scFv (20 mg/kg) 3×/week for 4 weeks.

FIG. 4 is a plot showing absolute radiance efficiencies of brain metastases tracked by IVIS in mice treated with tail vein injection of control (PBS) or h3E10 di-scFv (20 mg/kg) for 1 week or 4 weeks.

FIG. 5 is a bar graph showing hCMEC/D3 cells transfected with control or ENT2-targeting siRNA were treated with control buffer or h3E10 di-scFv alone, and then immunostained for h3E10 di-scFv and quantified by ImageJ. *P<0.01, n=3. Successful knockdown of ENT2 was confirmed by RT-PCR

FIG. 6 is a bar graph showing hCMEC/D3 BECs immunostained for h3E10 di-scFv after treatment h3E10 di-scFv alone (left), or h3E10 di-scFv+50 NM DP (right) and quantified by ImageJ. *P<0.01, n=3.

FIG. 7 is an illustration of a Transwell model/assay utilized in Example 2.

FIG. 8 is a bar graph quantifying an autoradiogram of Western blot images of h3E10 di-scFv content in basolateral chambers (+BBB) relative to control blank filters treated with h3E10 di-scFv (−BBB).

FIG. 9 is a line graph showing quantification of h3E10 di-scFv content in basolateral chambers at each time point, relative to the 30 minute time point in the absence of DP.

FIG. 10 is a dot plot quantifying the radiance efficacy measured in a series of IVIS image detecting h3E10 di-scFv (labeled with IR750) twenty-four hours after treatment with tail vein and intraperitoneal injection of control buffer (N=2), tail vein injection of h3E10 di-scFvIR750 and intraperitoneal injection of control buffer (N=4), or tail vein injection of h3E10 di-scFvIR750 and intraperitoneal injection of DP (70 mg/kg) (N=4).

### DETAILED DESCRIPTION OF THE INVENTION

### I. Definitions

As used herein, the term “single chain Fv” or “scFv” as used herein means a single chain variable fragment that includes a light chain variable region (V_{L}) and a heavy chain variable region (V_{H}) in a single polypeptide chain joined by a linker which enables the scFv to form the desired structure for antigen binding (i.e., for the V_{H} and V_{L} of the single polypeptide chain to associate with one another to form a Fv). The V_{L} and V_{H} regions may be derived from the parent antibody or may be chemically or recombinantly synthesized.

As used herein, the term “variable region” is intended to distinguish such domain of the immunoglobulin from domains that are broadly shared by antibodies (such as an antibody Fc domain). The variable region includes a “hypervariable region” whose residues are responsible for antigen binding. The hypervariable region includes amino acid residues from a “Complementarity Determining Region” or “CDR” (i.e., typically at approximately residues 24-34 (L1), 50-56 (L2) and 89-97 (L3) in the light chain variable domain and at approximately residues 27-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, MD. (1991)) and/or those residues from a “hypervariable loop” (i.e., residues 26-32 (L1), 50-52 (L2) and 91-96 (L3) in the light chain variable domain and 26-32 (H1), 53-55 (H2) and 96-101 (H3) in the heavy chain variable domain; Chothia and Lesk, 1987, J. Mol. Biol. 196:901-917).

As used herein, the term “Framework Region” or “FR” residues are those variable domain residues other than the hypervariable region residues as herein defined.

As used herein, the term “antibody” refers to natural or synthetic antibodies that bind a target antigen. The term includes polyclonal and monoclonal antibodies. In addition to intact immunoglobulin molecules, also included in the term “antibodies” are fragments or polymers of those immunoglobulin molecules, and human or humanized versions of immunoglobulin molecules that bind the target antigen.

The term “cell penetrating antibody” refers to an immunoglobulin protein, fragment, or variant thereof that is transported across the cell membrane (i.e., into the cytoplasm) of living mammalian cells. The antibody can be transported into the cytoplasm of the cells without the aid of a carrier or conjugate. In some embodiments, the antibody, fragment, or variant thereof is conjugated to a cell-penetrating moiety, such as a cell penetrating peptide.

As used herein, the term “nuclear penetrating antibody” refers to an antibody, or antigen binding fragment or molecule thereof that is transported into the nucleus of living mammalian cells and binds to a target therein (e.g., a nuclear localized ligand). Exemplary targets include, but are not limited proteins and nucleic acids. An antibody that binds to DNA (e.g., single-stranded and/or double-stranded DNA) can be referred to as an anti-DNA antibody. In some embodiments, a nuclear penetrating antibody is transported into the nucleus of a cell without the aid of a carrier or conjugate. In another embodiment, a nuclear penetrating antibody is conjugated to a cell and/or nuclear-penetrating moiety, such as a cell penetrating peptide. One of skill in the art will appreciate that the term “nuclear penetrating” can be used in the context of the present disclosure to refer to other particles having a targeting moiety that targets a nuclear ligand such as scFv. For example, the term can be used to refer to a scFv that is transported into the nucleus of a cell without the aid of a carrier or conjugate and binds a nuclear ligand (e.g., single-stranded and/or double-stranded DNA, RNA, protein, etc.).

As used herein, the term “specifically binds” refers to the binding of an antibody to its cognate antigen (for example DNA) while not significantly binding to other antigens. Preferably, an antibody “specifically binds” to an antigen with an affinity constant (Ka) greater than about 10⁵ mol⁻¹ (e.g., 10⁶ mol⁻¹, 10⁷ mol⁻¹, 10⁸ mol⁻¹, 10⁹ mol⁻¹, 10¹⁰ mol⁻¹, 10¹¹ mol⁻¹, and 10¹² mol⁻¹ or more) with that second molecule.

As used herein, the term “monoclonal antibody” or “MAb” refers to an antibody obtained from a substantially homogeneous population of antibodies, i.e., the individual antibodies within the population are identical except for possible naturally occurring mutations that may be present in a small subset of the antibody molecules.

As used herein, the term “DNA repair” refers to a collection of processes by which a cell identifies and corrects damage to DNA molecules. Single-strand defects are repaired by base excision repair (BER), nucleotide excision repair (NER), or mismatch repair (MMR). Double-strand breaks are repaired by non-homologous end joining (NHEJ), microhomology-mediated end joining (MMEJ), or homologous recombination. After DNA damage, cell cycle checkpoints are activated, which pause the cell cycle to give the cell time to repair the damage before continuing to divide. Checkpoint mediator proteins include BRCA1, MDC1, 53BP1, p53, ATM, ATR, CHK1, CHK2, and p21.

As used herein, the term “impaired DNA repair” refers to a state in which a mutated cell or a cell with altered gene expression is incapable of DNA repair or has reduced activity or efficiency of one or more DNA repair pathways or takes longer to repair damage to its DNA as compared to a wild type cell.

As used herein, the term “tumor” or “neoplasm” refers to an abnormal mass of tissue containing neoplastic cells. Neoplasms and tumors may be benign, premalignant, or malignant.

As used herein, the term “cancer” or “malignant neoplasm” refers to a cell that displays uncontrolled growth and division, invasion of adjacent tissues, and often metastasizes to other locations of the body.

As used herein, the term “inhibit” means to decrease an activity, response, condition, disease, or other biological parameter. This can include, but is not limited to, the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

As used herein, the term “fusion protein” refers to a polypeptide formed by the joining of two or more polypeptides through a peptide bond formed between the amino terminus of one polypeptide and the carboxyl terminus of another polypeptide or through linking of one polypeptide to another through reactions between amino acid side chains (for example disulfide bonds between cysteine residues on each polypeptide). The fusion protein can be formed by the chemical coupling of the constituent polypeptides or it can be expressed as a single polypeptide from a nucleic acid sequence encoding the single contiguous fusion protein. Fusion proteins can be prepared using conventional techniques in molecular biology to join the two genes in frame into a single nucleic acid sequence, and then expressing the nucleic acid in an appropriate host cell under conditions in which the fusion protein is produced.

As used herein, the term “variant” refers to a polypeptide or polynucleotide that differs from a reference polypeptide or polynucleotide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

Modifications and changes can be made in the structure of the polypeptides of in disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and cofactors. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly where the biological functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, 95%, 96%, 97%, 98%, 99%, or more sequence identity to the polypeptide of interest.

As used herein, the term “percent (%) sequence identity” is defined as the percentage of nucleotides or amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For purposes herein, the % sequence identity of a given nucleotides or amino acids sequence C to, with, or against a given nucleic acid sequence D (which can alternatively be phrased as a given sequence C that has or includes a certain % sequence identity to, with, or against a given sequence D) is calculated as follows:

100 times the fraction W/Z,

where W is the number of nucleotides or amino acids scored as identical matches by the sequence alignment program in that program's alignment of C and D, and where Z is the total number of nucleotides or amino acids in D. It will be appreciated that where the length of sequence C is not equal to the length of sequence D, the % sequence identity of C to D will not equal the % sequence identity of D to C.

As used herein, the term “sustained release” refers to release of a substance over an extended period of time in contrast to a bolus type administration in which the entire amount of the substance is made biologically available at one time.

As used herein, the phrase “pharmaceutically acceptable” refers to compositions, polymers and other materials and/or dosage forms which are, within the scope of sound medical judgment, suitable for use in contact with the tissues of human beings and animals without excessive toxicity, irritation, allergic response, or other adverse events, commensurate with a reasonable benefit/risk ratio.

As used herein, the phrase “pharmaceutically acceptable carrier” refers to pharmaceutically acceptable materials, compositions or vehicles, such as a liquid or solid filler, diluent, stabilizers, solvent or encapsulating matrix involved in carrying or transporting any subject composition, from one organ, or portion of the body, to another organ, or portion of the body. Each carrier must be “acceptable” in the sense of being compatible with the other ingredients of a subject composition and not injurious to the patient.

As used herein, the phrase “pharmaceutically acceptable salts” is art-recognized, and includes relatively non-toxic, inorganic and organic acid addition salts of compounds. Examples of pharmaceutically acceptable salts include those derived from mineral acids, such as hydrochloric acid and sulfuric acid, and those derived from organic acids, such as ethanesulfonic acid, benzenesulfonic acid, and p-toluenesulfonic acid. Examples of suitable inorganic bases for the formation of salts include the hydroxides, carbonates, and bicarbonates of ammonia, sodium, lithium, potassium, calcium, magnesium, aluminum, and zinc. Salts may also be formed with suitable organic bases, including those that are non-toxic and strong enough to form such salts.

As used herein, the term “individual,” “host,” “subject,” and “patient” are used interchangeably to refer to any individual who is the target of administration or treatment. The subject can be a vertebrate, for example, a mammal Thus, the subject can be a human or veterinary patient. In an example, the subject has an autoimmune disease such as lupus.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, “active agent” refers to a physiologically or pharmacologically active substance that acts locally and/or systemically in the body. An active agent is a substance that is administered to a patient for the treatment (e.g., therapeutic agent), prevention (e.g., prophylactic agent), or diagnosis (e.g., diagnostic agent) of a disease or disorder.

As used herein, the terms “effective amount” or “therapeutically effective amount” means a dosage sufficient to alleviate one or more symptoms of a disorder, disease, or condition being treated, or to otherwise provide a desired pharmacologic and/or physiologic effect. The precise dosage will vary according to a variety of factors such as subject-dependent variables (e.g., age, immune system health, weight, etc.), the disease or disorder being treated, as well as the route of administration, the pharmacokinetics of the agent being administered and the pharmacodynamic effects of the active.

As used herein, the term “prevention” or “preventing” means to administer a composition to a subject or a system at risk for or having a predisposition for one or more symptom caused by a disease or disorder to cause cessation of a particular symptom of the disease or disorder, a reduction or prevention of one or more symptoms of the disease or disorder, a reduction in the severity of the disease or disorder, the complete ablation of the disease or disorder, stabilization or delay of the development or progression of the disease or disorder.

The term “subject” or “patient” refers to any individual who is the target of administration. The subject can be a vertebrate, for example, a mammal. Thus, the subject can be a human. The subject can be domesticated, agricultural, or zoo- or circus-maintained animals. Domesticated animals include, for example, dogs, cats, rabbits, ferrets, guinea pigs, hamsters, pigs, monkeys or other primates, and gerbils. Agricultural animals include, for example, horses, mules, donkeys, burros, cattle, cows, pigs, sheep, and alligators. Zoo- or circus-maintained animals include, for example, lions, tigers, bears, camels, giraffes, hippopotamuses, and rhinoceroses. The term does not denote a particular age or sex. Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other forms the values may range in value either above or below the stated value in a range of approx. +/−5%; in other forms the values may range in value either above or below the stated value in a range of approx. +/−2%; in other forms the values may range in value either above or below the stated value in a range of approx. +/−1%. The ranges are intended to be made clear by context, and no further limitation is implied.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a ligand is disclosed and discussed and a number of modifications that can be made to a number of molecules including the ligand are discussed, each and every combination and permutation of ligand and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials.

These concepts apply to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

All methods described herein can be performed in any suitable order unless otherwise indicated or otherwise clearly contradicted by context. The use of any and all examples, or exemplary language (e.g., “such as”) provided herein, is intended merely to better illuminate the embodiments and does not pose a limitation on the scope of the embodiments unless otherwise claimed. No language in the specification should be construed as indicating any non-claimed element as essential to the practice of the invention.

### II. Compositions

The disclosed compositions typically are, or include, one or more nucleoside transporter inhibitors. In mammalian cells, nucleoside transporters are subdivided into two major classes based on their energy requirements; Na+-dependent secondary active concentrative transporters (CNTs) and Na+-independent equilibrative transporters (ENTs) (Wang, et al., Biochem Pharmacol. 2013; 86(11):1531-1540. doi:10.1016/j.bcp.2013.08.063, which is specifically incorporated by reference herein in its entirety).

In preferred embodiments, the inhibitor inhibits one or more ENTs. In some embodiments, the inhibitor inhibits only ENT2. Mammalian genomes encode 4 members of the equilibrative nucleoside transporter family that share the ability to transport adenosine and a likely 11-transmembrane (TM) helix topology, but differ in their abilities to transport other nucleosides and nucleobases. ENT1 and ENT2, are cell surface proteins that can regulate adenosine signaling. Human ENT1 (hENT1) transports a wide range of purine and pyrimidine nucleosides, but is unable to transport the pyrimidine base uracil. Human ENT2 (hENT2) exhibits similar substrate specificity to hENT1, although with a lower apparent affinity except in the case of inosine. It differs from hENT1 in also being able to transport purine and pyrimidine nucleobases. ENT3 has been shown to be a lysosomal transporter and functions as a pH-dependent transporter. ENT4, which was initially found to function as a polyspecific organic cation transporter and designated plasma membrane monoamine transporter (PMAT), was later shown to also transport adenosine at acidic pH.

In preferred embodiments, the inhibitor inhibits one or more of ENT1, ENT2, and ENT4, most preferably at least ENT2. In some embodiments, the inhibitor is specific for ENT2.

Several chemical classes have been shown to inhibit ENTs. Three classes of inhibitors include (1) purine nucleoside analogues of which NBMPR is the prototype, (2) pyrimidopyrimidine and pteridine derivatives such as dipyridamole, and (3) flazine calcium channel blockers represented by lidoflazine (Buolamwini, Curr Med Chem., 1997; 4:35-66, which is specifically incorporated by reference herein in its entirety). NBMPR is a potent inhibitor of hENT1 (IC50 of 0.4-8 nM) while it is a moderate inhibitor of hENT2 (IC50 of 2.8 μM) (Ward, et al. J Biol Chem. 2000; 275:8375-81, SenGupta, et al., Biochemistry-Us. 2002; 41:1512-9). Likewise, dipyridamole and dilazep inhibit hENT1 more potently (Ki values of 48 nM and 19 nM, respectively) than hENT2 (Ki values of 6.2 μM and 134 μM, respectively) (Visser, et al. J Biol Chem. 2002; 277:395-401). Both NBMPR and dipyridamole are moderate inhibitors of hENT4 (Barnes, et al., Circ Res. 2006; 99:510-9). Intranuclear transduction of mouse 3E10 Fv was shown to be dependent on ENT2, and inhibited by NBMPR (Weisbart et al., Sci Rep 5:12022. doi: 10.1038/srep12022. (2015), Zack et al., J Immunol 157, 2082-2088 (1996), Hansen et al., J Biol Chem 282, 20790-20793 (2007)).

NBMPR has immunosuppressive and mutagenic activities deriving from its 6-mercaptopurine metabolite (Benedict, et al., Cancer Res. 1977; 37:2209-13, Cass, et al., Cancer Res. 1975; 35:1187-93, Elion, et al., Symposium on immunosuppressive drugs. Biochemistry and pharmacology of purine analogues. Federation proceedings. 1967; 26:898). Flazines are nonspecific, having calcium channel antagonist activity, poor oral absorption and a short duration of action (Grover, et al., J Pharmacol Exp Ther. 1994; 268:90-6), Buolamwini, et al., Curr Med Chem. 1997; 4:35-66, Kates, et al., J Thorac Cardiovasc Surg. 1983; 85:278-86). Thus, in some embodiments, the inhibitor is not NBMPR, is not a flazine, or a not NBMPR or a flazine.

In preferred embodiments, the inhibitor is a pyrimidopyrimidine and pteridine derivatives such as dipyridamole or a tautomer, geometrical isomer, optically active form, enantiomeric mixture thereof, pharmaceutically acceptable salt or pharmaceutically active analogue thereof.

Dipyridamole has the structure:

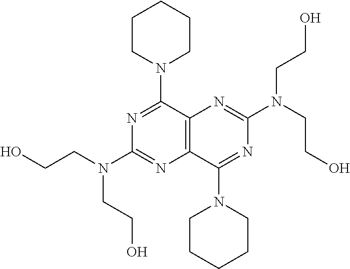 [SMILES: OCCN(CCO)c1nc(N2CCCCC2)c2nc(N(CCO)CCO)nc(N3CCCCC3)c2n1]

Dipyridamole (also referred to herein as DPM or DP) possesses beneficial properties and broad pharmacological effects (Wang, et al., Biochem Pharmacol. 2013; 86(11):1531-1540. doi:10.1016/j.bcp.2013.08.063). It is a safe drug that has been used for a long time in humans to prevent strokes and other vascular diseases due to its antiplatelet and vasodilating activities as a nucleoside transport inhibitor and a non-selective phosphodiesterase inhibitor (Jones, et al., Health Technol Assess. 2004; 8:iii-iv. 1-196, Chakrabarti, et al., Vascul Pharmacol. 2008; 48:143-9). Dipyridamole inhibits ENT1, ENT2 and ENT4, but blocks ENT1 better than ENT2 and only weakly inhibits ENT4.

Analogues of dipyridamole with various inhibitory activities toward ENTs are known in the art. See, for example, hENT1 of a series of dipyridamole analogs (Wang, et al., Biochem Pharmacol. 2013; 86(11):1531-1540. doi:10.1016/j.bcp.2013.08.063, Lin, et al., J Med Chem. 2007; 50:3906-20, Lin, et al., Bioconjug Chem. 2011; 22(6):1221-7, Curtin, et al., British Journal of Cancer (1999) 80(11), 1738-1746, WO 1998/043974, and U.S. Pat. No. 6,297,250, each of which is incorporated by reference.

For example, in some embodiments, the inhibitor is a pyrimidopyrimidine compound that modulates or inhibits transport of nucleosides or purines across cell membranes.

The compounds can have the general structural formula I

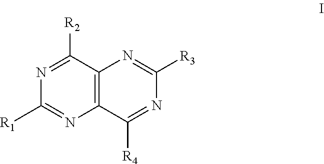

or pharmaceutically acceptable salts thereof,
characterized in that in structural formula I
R₁ is chloro and R₃ is diethanolamino, or
R₁ and R₃ are identical and are selected from allyl, halo, diethanolamino, solketalo and a group having the formula -0-R_{z} or —NHR_{z},
R_{z} being selected from alkyl, hydroxyalkyl, alkoxyalkyl, dialkoxyalkyl and 2-oxo-alkyl wherein the or each alkyl and/or alkoxy moiety has less than six carbon atoms, and
R₂ and R are identical and are selected from piperidino, N-tetrahydroisoquinolyl, and a benzylamino group having the structural formula II

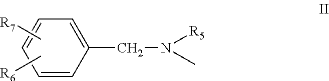

where
R₅ is H, or an optionally substituted alkyl or benzyl group, and
R₆ and R₇ represent H or optional substituents in the aromatic nucleus selected from halo, alkyl, alkoxy, hydroxy, trifluoromethyl, azido, cyano, nitro, carboxyl, carboxylic ester, amino or a substituted amino NRₓR_{y} where
Rₓ and R_{y} each represent hydrogen or alkyl, subject to the provisos that (a) if R₁ and R₃ are both chloro or diethanolamino,
R₂ and R₄ are not both benzylamino, i.e. R₂ and R₄ do not correspond to structure II with R₅, R₆ and R₇ each being hydrogen, and
(b) if R₂ and R are both piperidino, Rₓ and R₃ are not both chloro, diethanolamino, solketalo or (2,3-dtmethoxy) propoxy.

The term solketalo is used herein to denote the group 2,2-dimethyl-1,3-dtoxolane-4-methox.

When R₁ and/or R is halo, this will preferably be chloro.

Also, in some embodiments, when R₂ and R₄ are piperidino, R₁ and R₃ will be selected from allyl, methoxy, ethoxy, n-propoxy, iso-propoxy, iso-butoxy, 3-methylbutoxy, 2-oxo-n-propoxy, 2,2-diethoxy-n-propoxy, (2-methoxy-1-methyl) ethoxy, 2-methoxyethoxy, 2-hydroxy-propoxy, 2-hydroxyethoxy and 3-hydroxypropoxy; when R₂ and R₄ are N-tetrahydroisoquinolyl, R₁ and R₃ will each be selected from diethanolamino and chloro; and when R₂ and R₄ are each a benzylamino group of structural formulae II, R₁ and R₃ will be selected from 2-methoxyethoxy, propoxy, 2-hydroxypropoxy, diethanolamino, solketalo, chloro, 2-hydroxyethoxy and 3-hydroxypropoxy.

In some embodiments R₂ and R are in fact each provided by a benzylamino group of formula II, and preferably at least one of R₆ and R₇ will be alkoxy, especially methoxy, which will most preferably be substituted in the 4-position but which can alternatively or additionally (in disubstituted derivatives) be substituted in the 3-position. When R₅ is alkyl, it is preferably methyl.

In general, all alkyl groups when present as such, or as a moiety in other groups such as alkoxy, will be lower alkyl groups composed of 1-6 carbon atoms, preferably 1-5 carbon atoms, and more usually 1-4 carbon atoms with Ci, C₂ and C₃.

Within the series of dipyridamole analogue compounds wherein there are benzylamino groups conforming to structural formulae II, compounds of particular interest include compounds where the combination of substituents R₅, R₆ and R₇ is selected from the following combinations:

| R₅ | R₆ | R₇ |
|---|---|---|
| H | 4-OMe | H |
| Me | 4-OMe | H |
| H | 4-OMe | 3-OMe |
| Me | 4-OMe | 3-OMe |
| H | H | H |
| Me | H | H |
| benzyl | H | H |
| H | 4-CF₃ | H |

Examples of specific compounds within this series include:
(A1) 2,6-di-(3-hydroxypropoxy)-4,8-di-(N-4-methoxybenzyl-N-methylamino)pyrimido pyrimidine
(A2) 2,6-bis-diethanolamino-4,8-di-(4-methoxybenzylamino)pyrimidopyrimidine
(A3) 2,6-bis-diethanolamino-4,8-bis-(3,4-dimethoxybenzylamino)pyrimidopyrimidin e
(A4) 2,6-di-(2-hydroxypropoxy)-4,8-di-(4-methoxybenzylamino)pyrimidopyrimidine
(A5) 2,6-di(-3-hydroxypropoxy)-4,8-di(-4-methoxybenzylamino)pyrimidopyrimidine
(A6) 2,6-di-(2-hydroxyethoxy)-4,8-di-(N-4-methoxybenzyl-N-methylamino)pyrimidop yrimidine
(A7) 2-chloro-6-diethanolamino-4,8-bis[(3,4-dimethoxybenzyl)amino]pyrimidopyrim idine
(A8) 2,6-di(-3-hydroxypropoxy)-4,8-bis[(-3,4-dimethoxybenzyl)-N-methylamino]pyr imidopyrimidine
(A9) 2,6-bis(diethanolamino)-4,8-di(4-chlorobenzylamino)pyrimidopyrimidine
(A10) 2,6-di-(3-hydroxypropoxy)-4,8-di-(N-benzylmethylamino)pyrimidopyrimidine
(A11) 2,6-di(-2-hydroxyethoxy)-4,8-di(N-methylbenzylamino)pyrimidopyrimidine
(A12) 2,6-di(-2-methoxyethoxy)-4,8-dibenzylaminopyrimidopyrimidine
(A13) 2,6-di-(2-methoxyethoxy)-4,8-di-(N-benzyl-N-methylamino)pyrimidopyrimidine
(A14) 2,6-disolketalo-4,8-dibenzylaminopyrimidopyrimidine
(A15) 2,6-bis-diethanolamino-4,8-di-(4-trifluoromethylbenzylamino)pyrimidopyrimi dine
(A16) 2,6-di(-2-methoxyethoxy)-4,8-bis(dibenzylamino pyrimidopyrimidine
(A17) 2,6-dipropoxy-4,8-di-(N-benzyl-N-methylamino)pyrimidopyrimidine
(A18) 2,6-di(-2-hydroxyethyl)amino-4,8-di(4-methoxybenzyl)aminopyrimidopyrimidin e
(A19) 2,6-di(-2-hydroxyethylamino)-4,8-dibenzylaminopyrimidopyrimidine
(A20) 2,6-bis-diethanolamino-4,8-di-(N-methyl-N-[4-methoxybenzyl])aminopyrimidop yrimidine
(A21) 2,6-di-(2-hydroxyethyl)amino-4,8-bis-(3,4-dimethoxybenzyl)aminopyrimidopyr imidine
(A22) 2,6-Di-(3-hydroxypropoxy)-4,8-di-(N-[4-methoxybenzyl]-N-methyl)aminopyrimi dopyrimidine
(A23) 2,6-Di-(2-hydroxyethoxy)-4,8-di-(N-benzyl-N-methyl)aminopyrimidopyrimidine

It has been found that in this 4,8-dibenzylamino series of compounds, nucleoside transport inhibitory activity is usually enhanced by one or more alkoxy substituents, preferably methoxy substituents, and/or by N-methylation of the benzylamino group. Also, in this 4,8-dibenzylamino series at least one of R₁ and R₃ will usually be selected from diethanolamino, 2-hydroxypropoxy and 3-hydroxypropoxy in the most preferred compounds. When R₂ and R₄ are N-tetrahydroisoquinolyl, in a presently preferred embodiment R₁ and R₃ are both diethanolamino.

In the series of compounds in which R₂ and R₄ are provided by a piperidino group, compounds of particular interest include
(B1) (2-hydroxypropoxy)-4,8-dipiperidinopyrimidopyrimidine
(B2) (3-hydroxypropoxy)-4,8-dipiperidinopyrimidopyrimidine
(B3) (2-methoxyethoxy)-4,8-dipiperidinopyrimidopyrimidine
(B4) (2-hydroxyethoxy)-4,8-dipiperidinopyrimidopyrimidine
(B5) 2,6-bisl2,2-diethoxyl-n-propoxy-4,8-dipiperidinopyrimidopyrimidine
(B6) 2,6-di[2-oxo]-n-propoxy-4,8-dipiperidinopyrimidopyrimidine
(B7) 2,6-dimethoxy-4,8-dipiperidinopyrimidopyrimidine
(B8) 2,6-diethoxy-4,8-dipiperidinopyrimidopyrimidine
(B9) 2,6-di-3-methylbutoxy-4,8-dipiperidinopyrimidopyrimidine
(B10) (2-methoxy-1-methyl)ethoxy-4,8-dipiperidinopyrimidopyrimidine
(B11) 2,6-diallyl-4,8-dipiperidinopyrimidopyrimidine
(B12) 2,6-di-n-propoxy-4,8-dipiperidinopyrimidopyrimidine
(B13) 2,6-di-iso-propoxy-4,8-dipiperidinopyrimidopyrimidine
(B14) 2,6-di-iso-butoxy-4,8-dipiperidinopyrimidopyrimidine

Additional exemplary analogs are provided in the tables below:

TABLE 1
hENT1, 2 and 4 Inhibitory Activity of Dipyridamole Analogues
with different ring systems at the 4- and 8-positions (Wang, et al.,
Biochem Pharmacol. 2013;86(11):1531-1540.
doi:10.1016/j.bcp.2013.08.063)

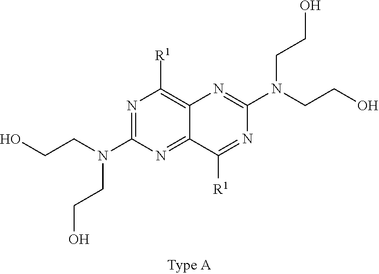

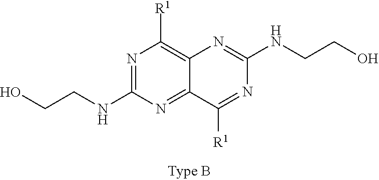

| Comp. (WL) | Type | R¹ | hENT1 IC₅₀ (nM) | hENT2 IC₅₀ (nM) | hENT4 IC₅₀ (nM) |
|---|---|---|---|---|---|
| NBMPR | — | — | 0.65 | 1,350 | 2330 |
| Dipyridamole | A | 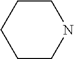 | 15.1 | 308 | 2797 |
| 1 | B | | 496 | 7,440 | 2196 |
| 2 | A | 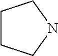 | 47.1 | 556 | — |
| 3 | B | | 115.9 | N.D.¹ | 3906 |
| 4 | A | 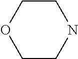 | 204.8 | 4,120 | N.D. |
| 5 | B | | 5,660 | N.D. | N.D. |
| 6 | A | 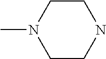 | N.D. | N.D. | N.D. |
| 7 | A | 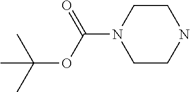 | 579.8 | 2,230 | N.D. |
| 8* | A | — | 937.1 | 3,660 | 12960 |
| 9 | A | 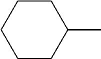 | 165.9 | N.D. | N.D. |
| 10 | A | 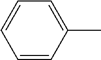 | 513.2 | 1,380 | 12000 |
| 11 | A | 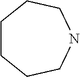 | 11.1 | 117 | 379 |
| 12 | B | | 465.7 | N.D. | 595 |
| 13 | A | 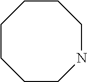 | 0.97 | 90.8 | 404 |
| 14 | B | | 217 | 5,200 | 181 |
| 15 | A | 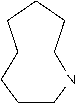 | 70.8 | 2,350 | 1320 |
| 16 | B | | 167.4 | 1,390 | 513 |
| 17 | A | 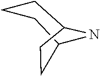 | 241.9 | 323 | — |
| 18 | B | | N.D. | N.D. | 324 |
| 19 | A | 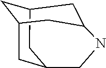 | 393 | N.D. | 200 |
| 20 | B | | 1,432 | N.D. | 602 |

¹N.D. not determined

TABLE 2
hENT1, 2 and 4 Inhibitory Activity of Dipyridamole Analogues
with opening chain analogues (Wang, et al., Biochem Pharmacol.
2013;86(11):1531-1540. doi:10.1016/j.bcp.2013.08.063)

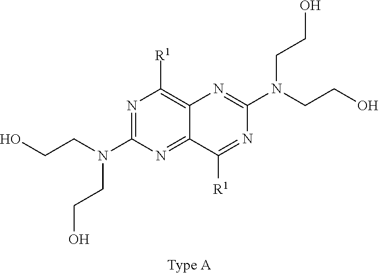

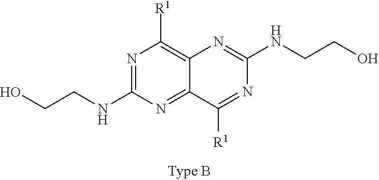

| Comp. (WL) | Type | R¹ | hENT1 IC₅₀ (nM) | hENT2 IC₅₀ (nM) | hENT4 IC₅₀ (nM) |
|---|---|---|---|---|---|
| 21 | A | Me₂N | 1,837 | 3,250 | 59566 |
| 22 | B | | N.D. | N.D. | — |
| 23 | A | Et₂N | 207.2 | 2,927 | 4121 |
| 24 | B | | 612.5 | 4,223 | 3130 |
| 25 | A | (n-Propyl)₂N | 197 | 3,830 | 234 |
| 26 | B | | 7,459 | N.D. | 97.7 |
| 27 | A | (n-Butyl)₂N | N.D. | 3,310 | 2960 |
| 28 | B | | N.D. | 1,312 | 871 |
| 29 | A | (iso-Butyl)₂N | 2,627 | 1,962 | 1710 |
| 30 | B | | 5,945 | 1,406 | 74.4 |
| 31 | A | (n-Pentyl)₂N | N.D. | N.D. | 45700 |
| 32 | B | | N.D. | N.D. | N.D. |
| 33 | A | (iso-Pentyl)₂N | N.D. | N.D. | 44200 |
| 34 | B | | N.D. | N.D. | — |
| 35 | A | 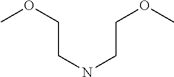 | 538.4 | 2,086 | 103514 |
| 36 | B | | 4,217 | 8,061 | 80352 |
| 37 | A | 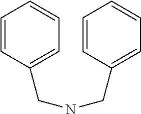 | N.D. | N.D. | 68400 |
| 38 | B | | N.D. | N.D. | N.D. |

TABLE 3
hENT1, 2 and 4 Inhibitory Activity of Dipyridamole Analogues
with free hydrogen on the nitrogen of 4- and 8-position substituents
(Wang, et al., Biochem Pharmacol. 2013;86(11):1531-1540.
doi:10.1016/j.bcp.2013.08.063)

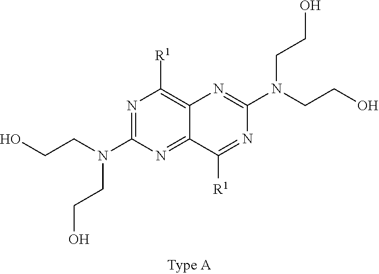

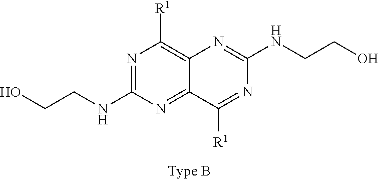

| Comp. (WL) | Type | R¹ | hENT1 IC₅₀ (nM) | hENT2 IC₅₀ (nM) | hENT4 IC₅₀ (nM) |
|---|---|---|---|---|---|
| 39 | A | NH₂ | N.D. | N.D. | N.D. |
| 40 | A | MeNH | 8,461 | N.D. | N.D. |
| 41 | B | | N.D. | N.D. | N.D. |
| 42 | A | EtNH | 768.3 | N.D. | N.D. |
| 43 | B | | N.D. | N.D. | 38459 |
| 44 | A | n-PropylNH | 427.7 | 3,500 | 16405 |
| 45 | B | | 3,877 | N.D. | 10115 |
| 46 | A | iso-PropylNH | 124.1 | 3,020 | 26002 |
| 47 | B | | 5,873 | N.D. | 11614 |
| 48 | A | n-ButylNH | 217 | 2,850 | 1883 |
| 49 | B | | 1,069 | 2,200 | 3870 |
| 50 | A | iso-ButylNH | 125.7 | 1,491 | 3055 |
| 51 | B | | 784.2 | 1,490 | 8660 |
| 52 | A | tert-ButylNH | 25.1 | N.D. | 767 |
| 53 | B | | 6,543 | N.D. | 3273 |
| 54 | A | n-PentylNH | 284.4 | 4,787 | 603 |
| 55 | B | | N.D. | N.D. | 3067 |
| 56 | A | iso-PentylNH | 142.6 | 1,954 | 495 |
| 57 | B | | 1,173 | N.D. | 3760 |
| 58 | A | tert-PentylNH | 78.9 | N.D. | 818 |
| 59 | B | | 2,607 | N.D. | 1339 |
| 60 | A | 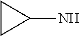 | 303.8 | 2,708 | 37600 |
| 61 | B | | N.D. | N.D. | N.D. |
| 62 | A | 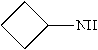 | 123.3 | 727.2 | 15668 |
| 63 | B | | 1,857 | 7,754 | — |
| 64 | A | 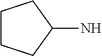 | 20.5 | 1,320 | 12162 |
| 65 | B | | N.D. | N.D. | N.D. |
| 66 | A | 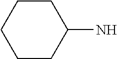 | 85.8 | N.D. | 124000 |
| 67 | B | | N.D. | N.D. | 65313 |
| 68 | A | 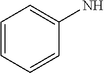 | 1,119 | N.D. | N.D. |
| 69 | B | | N.D. | N.D. | N.D. |
| 70 | A | 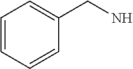 | N.D. | N.D. | N.D. |
| 71 | B | | N.D. | N.D. | 1760 |

TABLE 4
hENT1, 2 and 4 Inhibitory Activity of Dipyridamole Analogues
with modification at hydroxyl groups of dipyridamole (Wang, et al.,
Biochem Pharmacol. 2013;86(11):1531-1540.
doi:10.1016/j.bcp.2013.08.063)

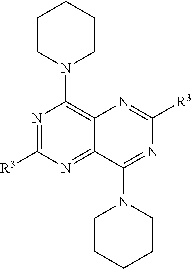

| Comp. (WL) | R³ | hENT1 IC₅₀ (nM) | hENT2 IC₅₀ (nM) | hENT4 IC₅₀ (nM) |
|---|---|---|---|---|
| 72 | OCH₂CH₂OH | 289.6 | 1,785 | — |
| 73 | 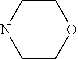 | 1,638 | N.D. | N.D. |
| 74 | N(CH₂CH₂OOCH)₂ | 69.1 | 532 | N.D. |
| 75 | N(CH₂CH₂OOCCH₃)₂ | 238 | 708 | 14125 |
| 76 | N(CH₂CH₂OCH₃)₂ | 207.7 | 1,470 | 1820 |
| 77 | N(CH₂CH₂OCH₂CH₃)₂ | 767.7 | N.D. | 29864 |
| 78 | N(CH₂CH₂OCH₂CH₂CH₃)₂ | N.D. | N.D. | 139637 |
| 79 | 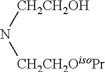 | 67.5 | 175 | N.D. |

TABLE 5
Dipyridamole analogues (Curtin, et al., British Journal of Cancer
(1999) 80(11), 1738-1746)

| Compound | Structure |
|---|---|
| Dipyridamole 2,6-bis-(diethanolamino)-4,8- di-piperidino- pyrimidopyrimidine | 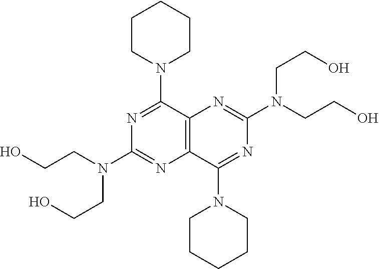 [SMILES: OCCN(CCO)c1nc(N2CCCCC2)c2nc(N(CCO)CCO)nc(N3CCCCC3)c2n1] |
| NU3026 (RR)^{c} 2,6-di-(2,2-dimethyl-1,3- dioxolan-4-yl)-methoxy- 4,8-di-piperidinopyrimido- pyrimidine | 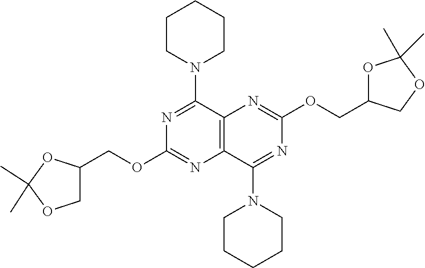 |
| NU3059 2,6-bis-(2,3-dimethoxypropoxy)- 4,8-di-piperidino- pyrimidopyrimidine | 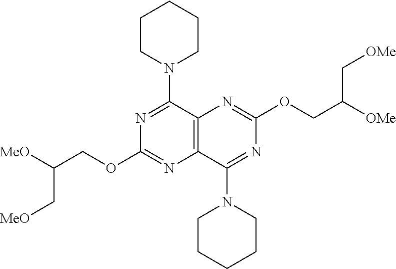 [SMILES: COCC(COc1nc(N2CCCCC2)c2nc(OCC(COC)OC)nc(N3CCCCC3)c2n1)OC] |
| NU3060 2,6-bis[N,N-di(2-methoxy)ethyl]- 4,8-di-piperidino- pyrimidopyrimidine | 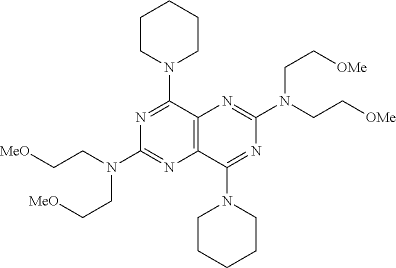 [SMILES: COCCN(CCOC)c1nc(N2CCCCC2)c2nc(N(CCOC)CCOC)nc(N3CCCCC3)c2n1] |

In some embodiments, the nucleoside transporter inhibitors is an oligonucleotide inhibitor. The oligonucleotide inhibitors can be designed to target and reduced expression or translation of one or more nucleic acids (e.g., DNA including genomic DNA, RNA including mRNA, etc.) encoding a nucleoside transporter. Exemplary oligonucleotide inhibitors include isolated or synthetic antisense RNA or DNA, siRNA or siDNA, miRNA, miRNA mimics, short hairpin RNA (shRNA) or DNA (shDNA) and Chimeric Antisense DNA or RNA. The term “antisense” as used herein means a sequence of nucleotides complementary to and therefore capable of binding to a coding sequence, which may be either that of the strand of a DNA double helix that undergoes transcription, or that of a messenger RNA molecule. The terms “short hairpin RNA” or “shRNA” refer to an RNA structure having a duplex region and a loop region. The term small interfering RNA (siRNA), sometimes known as short interfering RNA or silencing RNA, is a class of double-stranded RNA molecules, 20-25 base pairs in length. A siRNA that inhibits or prevents translation to a particular protein is indicated by the protein name coupled with the term siRNA. The term “microRNA” (abbreviated miRNA) is a small non-coding RNA molecule (containing about 22 nucleotides) found in plants, animals and some viruses, that functions in RNA silencing and post-transcriptional regulation of gene expression. The prefix “miR” is followed by a dash and a number, the latter often indicating order of naming Different miRNAs with nearly identical sequences except for one or two nucleotides are annotated with an additional lower case letter.

In other embodiments, the inhibitor is an antibody or an antigen binding fragment or fusion protein thereof binds to a nucleoside transporter such as one or more of ENT1, ENT2, ENT3, and/or ENT4.

### III. Formulations

The disclosed compounds can be formulated in a pharmaceutical composition. Pharmaceutical compositions can be for administration by parenteral (intramuscular, intraperitoneal, intravenous (IV) or subcutaneous injection), enteral, transdermal (either passively or using iontophoresis or electroporation), or transmucosal (nasal, pulmonary, vaginal, rectal, or sublingual) routes of administration or using bioerodible inserts and can be formulated in dosage forms appropriate for each route of administration.

The compositions can be administered systemically, regionally, or locally.

Drugs can be formulated for immediate release, extended release, or modified release. A delayed release dosage form is one that releases a drug (or drugs) at a time other than promptly after administration. An extended release dosage form is one that allows at least a two-fold reduction in dosing frequency as compared to that drug presented as a conventional dosage form (e.g. as a solution or prompt drug-releasing, conventional solid dosage form). A modified release dosage form is one for which the drug release characteristics of time course and/or location are chosen to accomplish therapeutic or convenience objectives not offered by conventional dosage forms such as solutions, ointments, or promptly dissolving dosage forms. Delayed release and extended release dosage forms and their combinations are types of modified release dosage forms.

Formulations are prepared using a pharmaceutically acceptable carrier composed of materials that are considered safe and effective and may be administered to an individual without causing undesirable biological side effects or unwanted interactions.

The compositions can include one or more excipients. Excipients are all components present in the pharmaceutical formulation other than the active pharmaceutical agent or agent(s) (i.e., nucleoside transporter inhibitors) being delivered. The term excipient includes, but is not limited to, diluents, binders, lubricants, disintegrants, fillers, and coating compositions. Excipient also includes all components of the coating composition which may include plasticizers, pigments, solubilizes, colorants, stabilizing agents, and glidants. The delayed release dosage formulations may be prepared as described in references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, M D, 2000, and “Pharmaceutical dosage forms and drug delivery systems”, 6th Edition, Ansel et. al., (Media, PA: Williams and Wilkins, 1995) which provides information on excipients, materials, equipment and process for preparing tablets and capsules and delayed release dosage forms of tablets, capsules, and granules. See also, Handbook Of Pharmaceutical Excipients, sixth edition, Ed. By Raymond, et al., (2009).

The compound can be administered to a subject with or without the aid of a delivery vehicle. Appropriate delivery vehicles for the compounds are known in the art and can be selected to suit the particular active agent. For example, in some embodiments, the active agent(s) and/or other pharmaceutical ingredient(s) is/are incorporated into or encapsulated by, conjugated to, or otherwise bound to, a nanoparticle, microparticle, micelle, polymeric micelle, polymersome, microbubble, liposome, synthetic lipoprotein particle, dendrimer, or carbon nanotube. For example, the compositions can be incorporated into a vehicle such as polymeric particles or conjugated to dendrimer(s) which provide controlled release of the active agent(s). In some embodiments, release of the drug(s) is controlled by diffusion of the active agent(s) out of the microparticles and/or degradation or erosion of the polymeric particles by hydrolysis, osmotic release, and/or enzymatic degradation. In some embodiments, composition is administered as in situ gel forming depot that releases the active agent.

Suitable polymers include ethylcellulose and other natural or synthetic cellulose derivatives. Polymers which are slowly soluble and form a gel in an aqueous environment, such as hydroxypropyl methylcellulose or polyethylene oxide, may also be suitable as materials for drug containing microparticles or particles. Other polymers include, but are not limited to, polyanhydrides, poly (ester anhydrides), polycaprolactones, polyhydroxy acids, such as polylactide (PLA), polyglycolide (PGA), poly(lactide-co-glycolide) (PLGA), poly-3-hydroxybut rate (PHB) and copolymers thereof, poly-4-hydroxybutyrate (P4HB) and copolymers thereof, polycaprolactone and copolymers thereof, and combinations thereof. In some embodiments, both agents are incorporated into the same particles and are formulated for release at different times and/or over different time periods. For example, in some embodiments, one of the agents is released entirely from the particles before release of the second agent begins. In other embodiments, release of the first agent begins followed by release of the second agent before the all of the first agent is released. In still other embodiments, both agents are released at the same time over the same period of time or over different periods of time.

A. Formulations for Parenteral Administration

Compounds and pharmaceutical compositions thereof can be administered in an aqueous solution, by parenteral injection. The formulation may also be in the form of a suspension or emulsion. In general, pharmaceutical compositions are provided including effective amounts of the active agent(s) and optionally include pharmaceutically acceptable diluents, preservatives, solubilizers, emulsifiers, adjuvants, excipients, and/or carriers. Such compositions include diluents sterile water, buffered saline of various buffer content (e.g., Tris-HCl, acetate, phosphate), pH and ionic strength; and optionally, additives such as detergents and solubilizing agents (e.g., TWEEN® 20, TWEEN® 80 also referred to as POLYSORBATE® 20 or 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, medium chain triglycerides (MCT), gelatin, and injectable organic esters such as ethyl oleate. The formulations may be lyophilized and reconstituted/resuspended immediately before use. The formulation may be sterilized by, for example, filtration through a bacteria retaining filter, by incorporating sterilizing agents into the compositions, or by irradiating the compositions.

B. Oral Immediate Release Formulations

Suitable oral dosage forms include tablets, capsules, solutions, suspensions, syrups, wafers, and lozenges. Tablets can be made using compression or molding techniques well known in the art. Gelatin or non-gelatin capsules can be prepared as hard or soft capsule shells, which can encapsulate liquid, solid, and semi-solid fill materials, using techniques well known in the art.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), Zein, shellac, and polysaccharides.

Additionally, the coating material may contain conventional carriers such as plasticizers, pigments, colorants, glidants, stabilization agents, pore formers and surfactants.

Optional pharmaceutically acceptable excipients present in the drug-containing tablets, beads, granules or particles include, but are not limited to, diluents, binders, lubricants, disintegrants, colorants, stabilizers, and surfactants.

Diluents, also termed “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powder sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydorxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp).

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, POLOXAMER® 401, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine and lauryl sulfobetaine.

If desired, the tablets, beads granules or particles may also contain minor amount of nontoxic auxiliary substances such as wetting or emulsifying agents, dyes, pH buffering agents, and preservatives.

C. Extended Release Dosage Forms

The extended release formulations can be prepared as diffusion or osmotic systems, for example, as described in “Remington—The science and practice of pharmacy” (20th ed., Lippincott Williams & Wilkins, Baltimore, MD, 2000). A diffusion system typically consists of two types of devices, reservoir and matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but not limited to, methyl acrylate-methyl methacrylate, polyvinyl chloride, and polyethylene. Hydrophilic polymers include, but are not limited to, methylcellulose, hydroxypropylcellulose, hydroxypropylmethylcellulose, sodium carboxymethylcellulose, and carbopol 934, polyethylene oxides. Fatty compounds include, but are not limited to, various waxes such as carnauba wax and glyceryl tristearate.

Alternatively, extended release formulations can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above could be combined in a final dosage form comprising single or multiple units. Examples of multiple units include multilayer tablets, capsules containing tablets, beads, granules, etc.

An immediate release portion can be added to the extended release system by means of either applying an immediate release layer on top of the extended release core using coating or compression process or in a multiple unit system such as a capsule containing extended and immediate release beads.

Extended release tablets containing hydrophilic polymers are prepared by techniques commonly known in the art such as direct compression, wet granulation, or dry granulation processes. Their formulations usually incorporate polymers, diluents, binders, and lubricants as well as the active pharmaceutical ingredient. The usual diluents include inert powdered substances such as any of many different kinds of starch, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidine can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In a congealing method, the drug is mixed with a wax material and either spray-congealed or congealed and screened and processed.

D. Delayed Release Dosage Forms Delayed release formulations are created by coating a solid dosage form with a film of a polymer which is insoluble in the acid environment of the stomach, and soluble in the neutral environment of small intestines.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename EUDRAGIT®. (Rohm Pharma; Westerstadt, Germany), including EUDRAGIT®. L30D-55 and L100-55 (soluble at pH 5.5 and above), EUDRAGIT®. L-100 (soluble at pH 6.0 and above), EUDRAGIT®. S (soluble at pH 7.0 and above, as a result of a higher degree of esterification), and EUDRAGITS®. NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

Methods of Manufacturing

As will be appreciated by those skilled in the art and as described in the pertinent texts and literature, a number of methods are available for preparing drug-containing tablets, beads, granules or particles that provide a variety of drug release profiles. Such methods include, but are not limited to, the following: coating a drug or drug-containing composition with an appropriate coating material, typically although not necessarily incorporating a polymeric material, increasing drug particle size, placing the drug within a matrix, and forming complexes of the drug with a suitable complexing agent.

The delayed release dosage units may be coated with the delayed release polymer coating using conventional techniques, e.g., using a conventional coating pan, an airless spray technique, fluidized bed coating equipment (with or without a Wurster insert). For detailed information concerning materials, equipment and processes for preparing tablets and delayed release dosage forms, see Pharmaceutical Dosage Forms: Tablets, eds. Lieberman et al. (New York: Marcel Dekker, Inc., 1989), and Ansel et al., Pharmaceutical Dosage Forms and Drug Delivery Systems, 6.sup.th Ed. (Media, PA: Williams & Wilkins, 1995).

A preferred method for preparing extended release tablets is by compressing a drug-containing blend, e.g., blend of granules, prepared using a direct blend, wet-granulation, or dry-granulation process. Extended release tablets may also be molded rather than compressed, starting with a moist material containing a suitable water-soluble lubricant. However, tablets are preferably manufactured using compression rather than molding. A preferred method for forming extended release drug-containing blend is to mix drug particles directly with one or more excipients such as diluents (or fillers), binders, disintegrants, lubricants, glidants, and colorants. As an alternative to direct blending, a drug-containing blend may be prepared by using wet-granulation or dry-granulation processes. Beads containing the active agent may also be prepared by any one of a number of conventional techniques, typically starting from a fluid dispersion. For example, a typical method for preparing drug-containing beads involves dispersing or dissolving the active agent in a coating suspension or solution containing pharmaceutical excipients such as polyvinylpyrrolidone, methylcellulose, talc, metallic stearates, silicone dioxide, plasticizers or the like. The admixture is used to coat a bead core such as a sugar sphere (or so-called “non-pareil”) having a size of approximately 60 to 20 mesh.

An alternative procedure for preparing drug beads is by blending drug with one or more pharmaceutically acceptable excipients, such as microcrystalline cellulose, lactose, cellulose, polyvinyl pyrrolidone, talc, magnesium stearate, a disintegrant, etc., extruding the blend, spheronizing the extrudate, drying and optionally coating to form the immediate release beads.

E. Formulations for Mucosal and Pulmonary Administration

Active agent(s) and compositions thereof can be formulated for pulmonary or mucosal administration. The administration can include delivery of the composition to the lungs, nasal, oral (sublingual, buccal), vaginal, or rectal mucosa. In a particular embodiment, the composition is formulated for and delivered to the subject sublingually.

In one embodiment, the compounds are formulated for pulmonary delivery, such as intranasal administration or oral inhalation. The respiratory tract is the structure involved in the exchange of gases between the atmosphere and the blood stream. The lungs are branching structures ultimately ending with the alveoli where the exchange of gases occurs. The alveolar surface area is the largest in the respiratory system and is where drug absorption occurs. The alveoli are covered by a thin epithelium without cilia or a mucus blanket and secrete surfactant phospholipids. The respiratory tract encompasses the upper airways, including the oropharynx and larynx, followed by the lower airways, which include the trachea followed by bifurcations into the bronchi and bronchioli. The upper and lower airways are called the conducting airways. The terminal bronchioli then divide into respiratory bronchiole, which then lead to the ultimate respiratory zone, the alveoli, or deep lung. The deep lung, or alveoli, is the primary target of inhaled therapeutic aerosols for systemic drug delivery.

Pulmonary administration of therapeutic compositions comprised of low molecular weight drugs has been observed, for example, beta-androgenic antagonists to treat asthma. Other therapeutic agents that are active in the lungs have been administered systemically and targeted via pulmonary absorption. Nasal delivery is considered to be a promising technique for administration of therapeutics for the following reasons: the nose has a large surface area available for drug absorption due to the coverage of the epithelial surface by numerous microvilli, the subepithelial layer is highly vascularized, the venous blood from the nose passes directly into the systemic circulation and therefore avoids the loss of drug by first-pass metabolism in the liver, it offers lower doses, more rapid attainment of therapeutic blood levels, quicker onset of pharmacological activity, fewer side effects, high total blood flow per cm³, porous endothelial basement membrane, and it is easily accessible.

The term aerosol as used herein refers to any preparation of a fine mist of particles, which can be in solution or a suspension, whether or not it is produced using a propellant. Aerosols can be produced using standard techniques, such as ultrasonication or high-pressure treatment.

Carriers for pulmonary formulations can be divided into those for dry powder formulations and for administration as solutions. Aerosols for the delivery of therapeutic agents to the respiratory tract are known in the art. For administration via the upper respiratory tract, the formulation can be formulated into a solution, e.g., water or isotonic saline, buffered or un-buffered, or as a suspension, for intranasal administration as drops or as a spray. Preferably, such solutions or suspensions are isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. For example, a representative nasal decongestant is described as being buffered to a pH of about 6.2. One skilled in the art can readily determine a suitable saline content and pH for an innocuous aqueous solution for nasal and/or upper respiratory administration.

Preferably, the aqueous solution is water, physiologically acceptable aqueous solutions containing salts and/or buffers, such as phosphate buffered saline (PBS), or any other aqueous solution acceptable for administration to an animal or human. Such solutions are well known to a person skilled in the art and include, but are not limited to, distilled water, de-ionized water, pure or ultrapure water, saline, phosphate-buffered saline (PBS). Other suitable aqueous vehicles include, but are not limited to, Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

In another embodiment, solvents that are low toxicity organic (i.e. nonaqueous) class 3 residual solvents, such as ethanol, acetone, ethyl acetate, tetrahydrofuran, ethyl ether, and propanol may be used for the formulations. The solvent is selected based on its ability to readily aerosolize the formulation. The solvent should not detrimentally react with the compounds. An appropriate solvent should be used that dissolves the compounds or forms a suspension of the compounds. The solvent should be sufficiently volatile to enable formation of an aerosol of the solution or suspension. Additional solvents or aerosolizing agents, such as freons, can be added as desired to increase the volatility of the solution or suspension.

In one embodiment, compositions may contain minor amounts of polymers, surfactants, or other excipients well known to those of the art. In this context, “minor amounts” means no excipients are present that might affect or mediate uptake of the compounds in the lungs and that the excipients that are present are present in amount that do not adversely affect uptake of compounds in the lungs.

Dry lipid powders can be directly dispersed in ethanol because of their hydrophobic character. For lipids stored in organic solvents such as chloroform, the desired quantity of solution is placed in a vial, and the chloroform is evaporated under a stream of nitrogen to form a dry thin film on the surface of a glass vial. The film swells easily when reconstituted with ethanol. To fully disperse the lipid molecules in the organic solvent, the suspension is sonicated. Nonaqueous suspensions of lipids can also be prepared in absolute ethanol using a reusable PARI LC Jet+ nebulizer (PARI Respiratory Equipment, Monterey, CA).

Dry powder formulations (“DPFs”) with large particle size have improved flowability characteristics, such as less aggregation, easier aerosolization, and potentially less phagocytosis. Dry powder aerosols for inhalation therapy are generally produced with mean diameters primarily in the range of less than 5 microns, although a preferred range is between one and ten microns in aerodynamic diameter. Large “carrier” particles (containing no drug) have been co-delivered with therapeutic aerosols to aid in achieving efficient aerosolization among other possible benefits.

Polymeric particles may be prepared using single and double emulsion solvent evaporation, spray drying, solvent extraction, solvent evaporation, phase separation, simple and complex coacervation, interfacial polymerization, and other methods well known to those of ordinary skill in the art. Particles may be made using methods for making microspheres or microcapsules known in the art. The preferred methods of manufacture are by spray drying and freeze drying, which entails using a solution containing the surfactant, spraying to form droplets of the desired size, and removing the solvent.

The particles may be fabricated with the appropriate material, surface roughness, diameter and tap density for localized delivery to selected regions of the respiratory tract such as the deep lung or upper airways. For example, higher density or larger particles may be used for upper airway delivery. Similarly, a mixture of different sized particles, provided with the same or different active agents may be administered to target different regions of the lung in one administration.

F. Topical and Transdermal Formulations

Transdermal formulations may also be prepared. These will typically be gels, ointments, lotions, sprays, or patches, all of which can be prepared using standard technology. In some embodiments additional or alternative formulations are administered topically or transdermally using microneedles. Transdermal formulations can include penetration enhancers.

A “gel” is a colloid in which the dispersed phase has combined with the continuous phase to produce a semisolid material, such as jelly.

An “oil” is a composition containing at least 95% wt of a lipophilic substance. Examples of lipophilic substances include but are not limited to naturally occurring and synthetic oils, fats, fatty acids, lecithins, triglycerides and combinations thereof.

A “continuous phase” refers to the liquid in which solids are suspended or droplets of another liquid are dispersed, and is sometimes called the external phase. This also refers to the fluid phase of a colloid within which solid or fluid particles are distributed. If the continuous phase is water (or another hydrophilic solvent), water-soluble or hydrophilic drugs will dissolve in the continuous phase (as opposed to being dispersed). In a multiphase formulation (e.g., an emulsion), the discreet phase is suspended or dispersed in the continuous phase.

An “emulsion” is a composition containing a mixture of non-miscible components homogenously blended together. In particular embodiments, the non-miscible components include a lipophilic component and an aqueous component. An emulsion is a preparation of one liquid dispersed as small droplets within a continuous (bulk) phase. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

“Emollients” are an externally applied agent that softens or soothes skin and are generally known in the art and listed in compendia, such as the “Handbook of Pharmaceutical Excipients”, 4^{th} Ed., Pharmaceutical Press, 2003. These include, without limitation, almond oil, castor oil, ceratonia extract, cetostearoyl alcohol, cetyl alcohol, cetyl esters wax, cholesterol, cottonseed oil, cyclomethicone, ethylene glycol palmitostearate, glycerin, glycerin monostearate, glyceryl monooleate, isopropyl myristate, isopropyl palmitate, lanolin, lecithin, light mineral oil, medium-chain triglycerides, mineral oil and lanolin alcohols, petrolatum, petrolatum and lanolin alcohols, soybean oil, starch, stearyl alcohol, sunflower oil, xylitol and combinations thereof. In one embodiment, the emollients are ethylhexylstearate and ethylhexyl palmitate.

“Surfactants” are surface-active agents that lower surface tension and thereby increase the emulsifying, foaming, dispersing, spreading and wetting properties of a product. Suitable non-ionic surfactants include emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In one embodiment, the non-ionic surfactant is stearyl alcohol.

“Emulsifiers” are surface active substances which promote the suspension of one liquid in another and promote the formation of a stable mixture, or emulsion, of oil and water. Common emulsifiers are: metallic soaps, certain animal and vegetable oils, and various polar compounds. Suitable emulsifiers include acacia, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In one embodiment, the emulsifier is glycerol stearate.

A “lotion” is a low- to medium-viscosity liquid formulation. A lotion can contain finely powdered substances that are in soluble in the dispersion medium through the use of suspending agents and dispersing agents. Alternatively, lotions can have as the dispersed phase liquid substances that are immiscible with the vehicle and are usually dispersed by means of emulsifying agents or other suitable stabilizers. In one embodiment, the lotion is in the form of an emulsion having a viscosity of between 100 and 1000 centistokes. The fluidity of lotions permits rapid and uniform application over a wide surface area. Lotions are typically intended to dry on the skin leaving a thin coat of their medicinal components on the skin's surface.

A “cream” is a viscous liquid or semi-solid emulsion of either the “oil-in-water” or “water-in-oil type”. Creams may contain emulsifying agents and/or other stabilizing agents. In one embodiment, the formulation is in the form of a cream having a viscosity of greater than 1000 centistokes, typically in the range of 20,000-50,000 centistokes. Creams are often time preferred over ointments as they are generally easier to spread and easier to remove.

An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. The oil phase may consist at least in part of a propellant, such as an HFA propellant. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

A sub-set of emulsions are the self-emulsifying drug delivery systems (SEDDS). These drug delivery systems are typically capsules (hard shell or soft shell) comprised of the drug dispersed or dissolved in a mixture of surfactant(s) and lipophillic liquids such as oils or other water immiscible liquids. When the capsule is exposed to an aqueous environment and the outer gelatin shell dissolves, contact between the aqueous medium and the capsule contents instantly generates very small emulsion droplets. These typically are in the size range of micelles or nanoparticles. No mixing force is required to generate the emulsion as is typically the case in emulsion formulation processes.

The basic difference between a cream and a lotion is the viscosity, which is dependent on the amount/use of various oils and the percentage of water used to prepare the formulations. Creams are typically thicker than lotions, may have various uses and often one uses more varied oils/butters, depending upon the desired effect upon the skin. In a cream formulation, the water-base percentage is about 60-75% and the oil-base is about 20-30% of the total, with the other percentages being the emulsifier agent, preservatives and additives for a total of 100%.

An “ointment” is a semisolid preparation containing an ointment base and optionally one or more active agents. Examples of suitable ointment bases include hydrocarbon bases (e.g., petrolatum, white petrolatum, yellow ointment, and mineral oil); absorption bases (hydrophilic petrolatum, anhydrous lanolin, lanolin, and cold cream); water-removable bases (e.g., hydrophilic ointment), and water-soluble bases (e.g., polyethylene glycol ointments). Pastes typically differ from ointments in that they contain a larger percentage of solids. Pastes are typically more absorptive and less greasy that ointments prepared with the same components.

A “gel” is a semisolid system containing dispersions of small or large molecules in a liquid vehicle that is rendered semisolid by the action of a thickening agent or polymeric material dissolved or suspended in the liquid vehicle. The liquid may include a lipophilic component, an aqueous component or both. Some emulsions may be gels or otherwise include a gel component. Some gels, however, are not emulsions because they do not contain a homogenized blend of immiscible components.

Suitable gelling agents include, but are not limited to, modified celluloses, such as hydroxypropyl cellulose and hydroxyethyl cellulose; Carbopol homopolymers and copolymers; and combinations thereof. Suitable solvents in the liquid vehicle include, but are not limited to, diglycol monoethyl ether; alklene glycols, such as propylene glycol; dimethyl isosorbide; alcohols, such as isopropyl alcohol and ethanol. The solvents are typically selected for their ability to dissolve the drug. Other additives, which improve the skin feel and/or emolliency of the formulation, may also be incorporated. Examples of such additives include, but are not limited, isopropyl myristate, ethyl acetate, C12-C15 alkyl benzoates, mineral oil, squalane, cyclomethicone, capric/caprylic triglycerides, and combinations thereof.

Foams consist of an emulsion in combination with a gaseous propellant. The gaseous propellant consists primarily of hydrofluoroalkanes (HFAs). Suitable propellants include HFAs such as 1,1,1,2-tetrafluoroethane (HFA 134a) and 1,1,1,2,3,3,3-heptafluoropropane (HFA 227), but mixtures and admixtures of these and other HFAs that are currently approved or may become approved for medical use are suitable. The propellants preferably are not hydrocarbon propellant gases which can produce flammable or explosive vapors during spraying. Furthermore, the compositions preferably contain no volatile alcohols, which can produce flammable or explosive vapors during use.

Buffers are used to control pH of a composition. Preferably, the buffers buffer the composition from a pH of about 4 to a pH of about 7.5, more preferably from a pH of about 4 to a pH of about 7, and most preferably from a pH of about 5 to a pH of about 7. In a preferred embodiment, the buffer is triethanolamine.

Preservatives can be used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, and thimerosal.

Additional agents that can be added to the formulation include penetration enhancers. In some embodiments, the penetration enhancer increases the solubility of the drug, improves transdermal delivery of the drug across the skin, in particular across the stratum corneum, or a combination thereof. Some penetration enhancers cause dermal irritation, dermal toxicity and dermal allergies. However, the more commonly used ones include urea, (carbonyldiamide), imidurea, N, N-diethylformamide, N-methyl-2-pyrrolidone, 1-dodecal-azacyclopheptane-2-one, calcium thioglycate, 2-pyrrolidone, N,N-diethyl-m-toluamide, oleic acid and its ester derivatives, such as methyl, ethyl, propyl, isopropyl, butyl, vinyl and glycerylmonooleate, sorbitan esters, such as sorbitan monolaurate and sorbitan monooleate, other fatty acid esters such as isopropyl laurate, isopropyl myristate, isopropyl palmitate, diisopropyl adipate, propylene glycol monolaurate, propylene glycol monooleatea and non-ionic detergents such as BRIJ® 76 (stearyl poly(10 oxyethylene ether), BRIJ® 78 (stearyl poly(20)oxyethylene ether), BRIJ® 96 (oleyl poly(10)oxyethylene ether), and BRIJ® 721 (stearyl poly (21) oxyethylene ether) (ICI Americas Inc. Corp.). Chemical penetrations and methods of increasing transdermal drug delivery are described in Inayat, et al., Tropical Journal of Pharmaceutical Research, 8(2):173-179 (2009) and Fox, et al., Molecules, 16:10507-10540 (2011). In some embodiments, the penetration enhancer is, or includes, an alcohol such ethanol, or others disclosed herein or known in the art.

Delivery of drugs by the transdermal route has been known for many years. Advantages of a transdermal drug delivery compared to other types of medication delivery such as oral, intravenous, intramuscular, etc., include avoidance of hepatic first pass metabolism, ability to discontinue administration by removal of the system, the ability to control drug delivery for a longer time than the usual gastrointestinal transit of oral dosage form, and the ability to modify the properties of the biological barrier to absorption.

Controlled release transdermal devices rely for their effect on delivery of a known flux of drug to the skin for a prolonged period of time, generally a day, several days, or a week. Two mechanisms are used to regulate the drug flux: either the drug is contained within a drug reservoir, which is separated from the skin of the wearer by a synthetic membrane, through which the drug diffuses; or the drug is held dissolved or suspended in a polymer matrix, through which the drug diffuses to the skin. Devices incorporating a reservoir will deliver a steady drug flux across the membrane as long as excess undissolved drug remains in the reservoir; matrix or monolithic devices are typically characterized by a falling drug flux with time, as the matrix layers closer to the skin are depleted of drug. Usually, reservoir patches include a porous membrane covering the reservoir of medication which can control release, while heat melting thin layers of medication embedded in the polymer matrix (e.g., the adhesive layer), can control release of drug from matrix or monolithic devices. Accordingly, the active agent can be released from a patch in a controlled fashion without necessarily being in a controlled release formulation.

Patches can include a liner which protects the patch during storage and is removed prior to use; drug or drug solution in direct contact with release liner; adhesive which serves to adhere the components of the patch together along with adhering the patch to the skin; one or more membranes, which can separate other layers, control the release of the drug from the reservoir and multi-layer patches, etc., and backing which protects the patch from the outer environment.

Common types of transdermal patches include, but are not limited to, single-layer drug-in-adhesive patches, wherein the adhesive layer contains the drug and serves to adhere the various layers of the patch together, along with the entire system to the skin, but is also responsible for the releasing of the drug; multi-layer drug-in-adhesive, wherein which is similar to a single-layer drug-in-adhesive patch, but contains multiple layers, for example, a layer for immediate release of the drug and another layer for control release of drug from the reservoir; reservoir patches wherein the drug layer is a liquid compartment containing a drug solution or suspension separated by the adhesive layer; matrix patches, wherein a drug layer of a semisolid matrix containing a drug solution or suspension which is surrounded and partially overlaid by the adhesive layer; and vapor patches, wherein an adhesive layer not only serves to adhere the various layers together but also to release vapor. Methods for making transdermal patches are described in U.S. Pat. Nos. 6,461,644, 6,676,961, 5,985,311, and 5,948,433.

### IV. Method of Use

A. Methods of Treatment

The results below show that compounds that inhibit nucleoside transport can reduce translocation of cell penetrating antibodies into the cytosol of cells, and/or transcellular transport through cells. Thus, such compounds can be administered to a subject in need thereof in an effective amount to reduce translocation antibodies into the cytosol of cells and/or reduce transcellular transport through cells, are provided. The subject is can be, for example, an animal such as a human, dog, cat, cattle, sheep, pig, etc. Typically, translocation and/or transcellular transport is reduced in an amount effective to reduce one or more symptoms or conditions caused by, or associated with, a cell-penetrating antibody or antibodies. The antibody or antibodies can be autoantibodies. The antibodies can be anti-DNA antibodies. Exemplary anti-DNA/anti-nucleosome antibodies are known in the art (see, e.g., Shuster A. M. et. al., Science, v. 256, 1992, pp. 665-667, Isenberg, et al., Rheumatology, 46 (7):1052-1056 (2007))). For example, autoantibodies to single or double stranded deoxyribonucleic acid (DNA) are frequently identified in the serum of patients with systemic lupus erythematosus (SLE) and are often implicated in disease pathogenesis. The anti-bodies can be, but need not be, nuclear penetrating antibodies (e.g., anti-nuclear antibodies).

The mechanisms of cellular internalization by autoantibodies are diverse. Some are taken into cells through electrostatic interactions or FcR-mediated endocytosis, while others utilize mechanisms based on association with cell surface myosin or calreticulin, followed by endocytosis (Ying-Chyi et al., Eur J Immunol 38, 3178-3190 (2008), Yanase et al., J Clin Invest 100, 25-31 (1997)). Mouse 3E10 penetrates cells in an Fc-independent mechanism as evidenced by the ability of 3E10 fragments lacking an Fc to penetrate cells (Weisbart et al., Sci Rep 5:12022. doi: 10.1038/srep12022. (2015), Zack et al., J Immunol 157, 2082-2088 (1996), Hansen et al., J Biol Chem 282, 20790-20793 (2007)). Thus, typically the subject treated according to the disclosed methods have (e.g., an autoantibody) or will be administered (e.g., a therapeutic antibody) at least one antibody that utilizes a nucleoside transporter for cell transduction and/or transcellular transport, for which reduced cell transduction and/or transcellular transport is desired.

The precise dosage will vary according to a variety of factors such as subject-dependent variables (e.g., age, immune system health, clinical symptoms etc.). Exemplary dosages, symptoms, pharmacologic, and physiologic effects are discussed in more detail below.

In some embodiments, the compounds are administered in bolus, pulsatile, delayed release, dosage escalation, or dosage de-escalation fashion.

1. Exemplary Dosages

Typically, the disclosed compositions are administered in an effective therapeutic dose that can be between, for example, 100 μg and 5 g, or between 100 μg and 3 g, between 100 μg and 1 g, or between 150 μg and 500 mg, or between 200 μg and 200 mg per day, or between 1 mg and 35 mg, or between 2 mg and 35 mg, or between 3 mg and 20 mg.

For example, dipyridamole is commercially available in, e.g., tablets for oral administration, contains 25 mg, 50 mg, or 75 mg dipyridamole. The recommended dose is 75 to 100 mg four times daily when used as adjunctive in prophylaxis of thromboembolism after cardiac valve replacement (Dipyridamole FDA prescribing information).

In some embodiments, the disclosed methods include administration of similar dosages to those known for treating other diseases and conditions such as those discussed above. In some embodiments, the dosages are different.

2. Combination Therapy

In some embodiments, the inhibitor is administered in combination with one or more additional active agents. The combination therapies can include administration of the active agents together in the same admixture, or in separate admixtures. Therefore, in some embodiments, the pharmaceutical composition includes two, three, or more active agents. Such formulations typically include an effective amount of nucleoside transporter inhibitor. The different active agents can have the same or different mechanisms of action. In some embodiments, the combination results in an additive effect on the treatment of the disease or disorder. In some embodiments, the combinations results in a more than additive effect on the treatment of the disease or disorder.

The pharmaceutical compositions can be formulated as a pharmaceutical dosage unit, also referred to as a unit dosage form, which can include a single effective dose of nucleoside transporter inhibitor. Exemplary combination therapies are discussed in more detail below.

B. Diseases and Disorders to be Treated

Nuclear penetrating antibodies are believed to play a role in various autoimmune disorders such as systemic lupus erythematosus and scleroderma (e.g. Mok and Lau J Clin Pathol. 56:481-490 (2003); DeFranco, Immunol Cell Biol., 94(10): 918-924 (2016); Silosi, et al., Rom J Morphol Embryol, 57(2 Suppl):633-638 (2016)). The experiments discussed in more detail below indicate that inhibition of a nucleoside transporter such as ENT2 may provide an effective means for treating these disorders. Thus, the disclosed compositions and methods can be used to treat autoimmune diseases, particularly autoimmune diseases that have symptoms or pathology dependent on or otherwise caused by cell penetration or transcellular transport of antibodies, for example, cell penetrating and/or transcellular transported autoantibodies.

Autoantibodies are responsible for disease manifestations in a variety of autoimmune diseases, including, systemic lupus erythematosus (lupus or SLE), systemic sclerosis (scleroderma), Graves' disease, myastenia gravis, autoimmune hemolytic anemia, and pemphigus vulgaris, and additionally may contribute to the severity of disease in other autoimmune diseases such as rheumatoid arthritis (DeFranco, Immunol Cell Biol., 94(10): 918-924 (2016)). Other diseases with an autoimmune component include, but are not limited to, primary biliary cirrhosis, primary sclerosing cholangitis, psoriasis, psoriatic arthritis, POEMS syndrome, dermatomyositis, inclusion body myositis, inflammatory myopathies, vasculitis syndromes including but not limited to Churg-Strauss Syndrome, Wegener granulomatosis, Behcet's disease, Buerger's disease, Kawasaki disease, Takayasu's arteritis, Henoch-Schonlein purpura, Giant cell arteritis, and polyarteritis nodosa.

Autoimmune diseases can be mediated principally by autoantibodies or a combination of autoantibodies and T lymphocytes (i.e., non-principal diseases), and can be organ-specific or systemic (Silosi, et al., Rom J Morphol Embryol, 57(2 Suppl):633-638 (2016)). Thus, in some embodiments, the compositions and methods are used to treat a principal organ-specific autoimmune disease, a principal specific autoimmune disease, a non-principal organ-specific autoimmune disease, or a non-principal specific autoimmune disease.

Exemplary principal organ-specific autoimmune diseases include, but are not limited to, autoimmune hemolytic anemia, autoimmune thrombocytopenia, autoimmune atrophic gastritis of pernicious anemia, myastenia gravis; and Goodpasture's syndrome.

Exemplary principal systemic disease autoimmune diseases include, but are not limited to, systemic lupus erythematosus (lupus or SLE).

In some embodiments, the subject has nephritis and the method reduces the nephritis, or prevents advancement of the nephritis. Nephritis is inflammation of the kidneys and may involve the glomeruli, tubules, or interstitial tissue surrounding the glomeruli and tubules. Nephritis is often caused by infections, and toxins, but is often caused by autoimmune disorders, such as SLE, that affect the major organs like kidneys. In some embodiments, the subject has lupus nephritis. Preferably the method improves kidney function, particularly in subjects with lupus.

Exemplary non-principal organ-specific disease autoimmune diseases involving both T lymphocytes and antibodies include, but are not limited to, diabetes mellitus, multiple sclerosis, Hashimoto's thyroiditis, and Crohn's disease.

Exemplary non-principal systemic disease autoimmune diseases involving both T lymphocytes and antibodies include, but are not limited to, rheumatoid arthritis, systemic sclerosis, and Sjögren's syndrome.

In organ-specific autoimmune diseases (such as myasthenia gravis or pemphigus), autoantibodies directly bind to and injure target organs. In some diseases, the autoimmune aggression results in the complete and irreversible loss of function of the targeted tissue (e.g., Hashimoto's thyroiditis or insulin-dependent diabetes). The autoimmune reactions may cause persistent lesions inducing an overstimulation or inhibition of its function (e.g., Graves-Basedow disease or myasthenia gravis). In other autoimmune conditions, the pathogenic events are multiple and produce destruction of several tissues (e.g., SLE).

1. Lupus

In some embodiments, the compositions and methods are used to treat a type or form of lupus.

Lupus is a chronic inflammatory disease that can affect many different parts of the body, and can cause damage to, for example, the skin, joints, kidneys, lungs, blood cells, heart, and brain. People with lupus may experience periods of flares when symptoms show up, and periods of remission when symptoms are under control. During a lupus flare, symptoms such as exhaustion, weight loss, fever, and anemia often occur. Lupus can cause damage to many parts of the body, potentially leading to the following complications: kidney failure, blood problems, such as anemia (low red blood cell count), bleeding, or clotting, high blood pressure, vasculitis (inflammation of the blood vessels), memory problems, behavior changes or hallucinations, seizures, stroke, heart disease or heart attack, lung conditions, such as pleurisy (inflammation of the chest cavity lining) or pneumonia, infections, cancer, and avascular necrosis (death of bone tissue due to a lack of blood supply).

Types of lupus include, systemic lupus erythematosus, or SLE (which is the most common form of lupus), discoid lupus erythematosus (which leads to a skin rash), subacute cutaneous lupus erythematosus (which leads to skin sores on areas of the body exposed to the sun), neonatal lupus (which affects newborns), and drug-induced lupus (which can be caused by certain medicines).

The presence of circulating autoantibodies reactive against DNA (anti-DNA antibodies) is a hallmark laboratory finding in patients with systemic lupus erythematosus (SLE). Although the precise role of anti-DNA antibodies in SLE is unclear, it has been proposed that the antibodies play an active role in SLE pathophysiology. Select lupus anti-DNA autoantibodies can penetrate into live cell nuclei and inhibit DNA repair or directly damage DNA.

In some embodiments, the subject has central nervous system (CNS) lupus. CNS lupus refers to several different neurological and/or behavioral clinical syndromes in patients with systemic lupus erythematosus (SLE) (Venuturupalli and Metzger, “CNS Lupus: Neurologic and psychiatric manifestations of Systemic Lupus Erythematosus,” Lupus International, (2011). The neuropsychiatric manifestations of lupus can vary from mild to severe and are often difficult to distinguish from other conditions and etiologies. Any location within the central nervous system (e.g., brain and spinal cord) may be affected with a variety of presentations from mild cognitive dysfunction to seizures, stroke or coma.

A variety of pathological processes may be involved in CNS lupus. The blood supply to a location or locations in the brain can be disturbed by autoimmune vasculitis (blood vessel inflammation), or clots formed as a result of antiphospholipid antibodies, or emboli originating from a cardiac source. Some subjects experience hyperviscosity, which may disrupt blood flow. Anti-neuronal antibodies also may be produced in some lupus patients, and these may have direct effects on the cells of the brain (neurons) and alter their function. The choroid plexus, a part of the brain that is the source of cerebrospinal fluid (CSF—a fluid bathing brain and spinal cord) may be involved thus causing diffuse problems. Several cytokines such as interleukin-1, interleukin-6 and interferon-γ are increased in CNS lupus and these have a direct effect on the neurons and can interfere with their function. Alterations in hormones produced in the hypothalamus, pituitary and adrenal glands (the HPA axis) are common in lupus and can cause some of the CNS perturbations. A number of secondary factors, including, for example, infection (lupus patients are more prone to certain types of infections), medications (several drugs such as corticosteroids have significant CNS toxicity), hypertension, electrolyte imbalances, uremia (renal failure), thyroid disease, atherosclerotic strokes, subdural hematomas, and fibromyalgia may also contribute to CNS-related symptoms.

The experiments below illustrate that dipyridamole reduces translocation of h3E10 di-scFv across the blood-brain barrier. Thus, dipyridamole and other nucleoside transporter inhibitors, particularly ENT2 nucleoside transporter inhibitors may be particular effective for reducing or prevent CNS lupus, or symptoms thereof. This is particularly true where one or more of the symptoms of the CNS lupus is tied to one or more autoantibodies that are dependent on a nucleoside transporter such as ENT2 to infiltration into the brain.

In some embodiments, the subject is administered a compound that inhibits a nucleoside transporter in combination with one or more additional active agents traditionally used to treat lupus. Traditional treatment for lupus includes non-steroidal anti-inflammatory drugs (NSAIDs), corticosteroids, immunosuppressants, hydroxychloroquine, and methotrexate. Disease-modifying antirheumatic drugs (DMARDs) are used preventively to reduce the incidence of flares, the progress of the disease, and the need for steroid use. When flares occur, they can be treated with corticosteroids. DMARDs commonly in use include antimalarials such as hydroxychloroquine and immunosuppressants (e.g. methotrexate and azathioprine). In more severe cases, immune modulators (such as corticosteroids and immunosuppressants) can be used to control the disease and prevent recurrence of symptoms. Steroid usage may lead a subject to develop Cushing's syndrome, symptoms of which may include obesity, puffy round face, diabetes mellitus, increased appetite, difficulty sleeping and osteoporosis. Subjects can also experience chronic pain, leading to administration of prescription analgesics including opioids if over-the-counter NSAIDs are insufficient. Intravenous immunoglobulins can be used to control SLE with organ involvement, or vasculitis. It is believed that they reduce antibody production or promote the clearance of immune complexes from the body, even though their mechanism of action is not well understood.

Having lupus can increase an individual's risk for cancer. Thus, in some embodiments, the subject has both an autoimmune disease such as lupus and a cancer. In some embodiments, the subject is administered a compound that inhibits a nucleoside transporter in combination with one or more anti-cancer agents. Additional therapeutic agents include conventional cancer therapeutics such as chemotherapeutic agents, cytokines, chemokines, and radiation therapy. The majority of chemotherapeutic drugs can be divided in to: alkylating agents, antimetabolites, anthracyclines, plant alkaloids, topoisomerase inhibitors, and other antitumor agents. All of these drugs affect cell division or DNA synthesis and function in some way. Additional therapeutics include monoclonal antibodies and the new tyrosine kinase inhibitors e.g. imatinib mesylate (GLEEVEC® or GLIVEC®), which directly targets a molecular abnormality in certain types of cancer (chronic myelogenous leukemia, gastrointestinal stromal tumors).

Representative chemotherapeutic agents include, but are not limited to cisplatin, carboplatin, oxaliplatin, mechlorethamine, cyclophosphamide, chlorambucil, vincristine, vinblastine, vinorelbine, vindesine, taxol and derivatives thereof, irinotecan, topotecan, amsacrine, etoposide, etoposide phosphate, teniposide, epipodophyllotoxins, trastuzumab (HERCEPTIN®), cetuximab, and rituximab (RITUXAN® or MABTHERA®), bevacizumab (AVASTIN®), nivolumab, ipilimumab, pemrolizumab, immune checkpoint inhibitors, and combinations thereof.

2. Scleroderma

In some embodiments, the compositions and methods are used to treat a form or type of scleroderma.

Scleroderma is a chronic connective tissue disease generally classified as one of the autoimmune rheumatic diseases. Patients with scleroderma can have specific antibodies (ANA, anticentromere, or antitopoisomerase) in their blood that suggest autoimmunity. Symptoms can generally include thickened skin that can involve scarring, blood vessel problems, varying degrees of inflammation and pain, and is associated with an overactive immune system.

Scleroderma can be classified in terms of the degree and location of the skin and organ involvement. Accordingly, scleroderma has been categorized into two major groups, localized scleroderma and systemic sclerosis, which can be further subdivided into either diffuse or limited forms based on the location and extent of skin involvement. Localized scleroderma skin changes are in isolated areas, either as morphea patches or linear scleroderma. Morphea is scleroderma that is localized to a patchy area of the skin that becomes hardened and slightly pigmented. Sometimes morphea can cause multiple lesions in the skin. Morphea is not associated with disease elsewhere within the body, only in the involved skin areas. Linear scleroderma is scleroderma that is localized usually to a lower extremity, frequently presenting as a strip of hardening skin down the leg of a child. Linear scleroderma in children can stunt bone growth of the affected limb. Sometimes linear scleroderma is associated with a “satellite” area of a patch of localized scleroderma skin, such as on the abdomen.

The widespread type of scleroderma involves internal organs in addition to the skin. This type, called systemic sclerosis, is subcategorized by the extent of skin involvement as either diffuse or limited. The diffuse form of scleroderma (diffuse systemic sclerosis) involves symmetric thickening of skin of the extremities, face, and trunk (chest, back, abdomen, or flanks) that can rapidly progress to hardening after an early inflammatory phase. Organ disease can occur early on and be serious and significantly decrease life expectancy. Organs affected include the esophagus, bowels, and scarring (fibrosis) of the lungs, heart, and kidneys. High blood pressure can be troublesome and can lead to kidney failure (renal crisis).

The limited form of scleroderma tends to have far less skin involvement with skin thickening confined to the skin of the fingers, hands, and face. The skin changes and other features of disease tend to occur more slowly than in the diffuse form. Because characteristic clinical features can occur in patients with the limited form of scleroderma, this form has taken another name that is composed of the first initials of the common components. Thus, this form is also called the “CREST” variant (subset thereof, e.g., CRST, REST, or ST) of scleroderma. CREST syndrome represents the following features: Calcinosis (the formation of tiny deposits of calcium in the skin), Raynaud's phenomenon (the spasm of the tiny arterial vessels supplying blood to the fingers, toes, nose, tongue, or ears), Esophagus disease (characterized by poorly functioning muscle of the lower two-thirds of the esophagus), Sclerodactyly (localized thickening and tightness of the skin of the fingers or toes), and Telangiectasias (tiny red areas, frequently on the face, hands, and in the mouth behind the lips).

Some subjects have scleroderma and one or more other connective tissue diseases, such as rheumatoid arthritis, systemic lupus erythematosus, and polymyositis. Features of scleroderma along with features of polymyositis, systemic lupus erythematosus, and certain abnormal blood tests, can lead to a diagnosis of mixed connective tissue disease (MCTD).

In some embodiments, the subject is administered a compound that inhibits a nucleoside transporter in combination with one or more additional active agents traditionally used to treat scleroderma. Current therapies use medications that focus on the four main features of the disease: inflammation, autoimmunity, vascular disease, and tissue fibrosis. Thus, subjects with scleroderma may be administered one or more anti-inflammatory agents, immunosuppressants, therapies for treating vascular disease, and/or anti-fibrotic agents. Anti-inflammatory medication include, but are not limited to, NSAIDs (e.g. ibuprofen) or corticosteroids (e.g. prednisone) Immunosuppressants include, but are not limited to, methotrexate, cyclosporine, antithymocyte globulin, mycophenolate mofetil and cyclophosphamide. Agents for treatment of vascular disease include, but are not limited to, vasodilators e.g., calcium channel blockers such as nifedipine, bosentan (endothelin-1 receptor inhibitor) and epoprostenol (prostacyclin) which can improve blood flow; agents which can reverse vasospasm such as angiotensin converting enzyme inhibitors (ACE) inhibitors, calcium channel blockers, bosentan, prostacyclin, or nitric oxide; and antiplatelet or anticoagulation therapy such as low-dose aspirin. Anti-fibrotic agents include, but are not limited to, colchicine, para-aminobenzoic acid (PABA), dimethyl sulfoxide, and D-penicillamine.

C. Methods of Modulating Anti-DNA Antibody Therapy

Select anti-DNA antibodies can penetrate into live cell nuclei and inhibit DNA repair or directly damage DNA, and efforts to use these antibodies against tumors that are sensitive to DNA damage are underway (Hansen, et al., Sci Transl Med, 4(157):157ra142 (2012), Noble, et al., Cancer Research, 2015; 75(11):2285-2291, Noble, et al., Sci Rep-Uk, 4 (2014), Noble, et al., Nat Rev Rheumatol (2016)).

A panel of hybridomas, including the 3E10 and 5C6 hybridomas was previously generated from the MRLmpj/lpr lupus mouse model and DNA binding activity was evaluated (Zack, et al., J. Immunol. 154:1987-1994 (1995); Gu, et al., J. Immunol., 161:6999-7006 (1998)). Thus in some embodiments, the anti-DNA antibody is 3E10 or 5C6 antibody or a variant, fragment, and fusion protein thereof, or a humanized form thereof.

In specific embodiments, a subject with cancer is administered a compound that inhibits a nucleoside transporter such as dipyridamole or a analogue thereof in combination with a cell penetrating and/or transcellular transportable antibody, or more particularly an anti-DNA antibody such as a 3E10 or 5C6 antibody or fragment or variant or humanized form thereof. 3E10 antibodies are attracted to tumors (see, e.g., WO 2017/218825), whereas systemically administered a nucleoside transporter such as dipyridamole or a analogue thereof can have a wider biodistribution. Thus, a nucleoside transporter such as dipyridamole or a analogue thereof can be used to tune the activity of an anti-DNA antibody such as 3E10 antibody or fragment or variant thereof, by reducing cellular penetration and/or transcellular transport of non-tumor tissues and further drive equilibrium of antibody or fragment or variant thereof towards tumors.

In some embodiments, the inhibitor is administered to a subject in an effective amount to reduce infiltration of the antibody or fragment or variant thereof into the brain, e.g., by reducing transport of the antibody or fragment or variant thereof across the BBB. Such subjects may have a non-CNS and/or non-brain cancer. The inhibitor reduces infiltration of the antibody or fragment or variant thereof into the brain while allowing it to target and act upon non-CNS and/or non-brain tumors.

An exemplary method includes administering to a subject with cancer an effective amount of a nucleoside transporter such as dipyridamole or a analogue thereof to reduce cell penetration and/or transcellular transport of a therapeutic antibody or variant or fragment or fusion protein thereof, without eliminating the ability of the antibody to treat the cancer.

Cell-penetrating antibodies and binding proteins that can induce DNA damage and/or reduce or impair DNA damage repair are known in the art. For example, select lupus anti-DNA autoantibodies can penetrate into live cell nuclei and impair DNA repair or directly damage DNA, and efforts to use these antibodies against tumors that are sensitive to DNA damage are underway (Hansen, et al., Sci Transl Med, 4(157):157ra142 (2012), Noble, et al., Cancer Research, 2015; 75(11):2285-2291, Noble, et al., Sci Rep-Uk, 4 (2014), Noble, et al., Nat Rev Rheumatol (2016)). Therefore, in some embodiments, anti-DNA antibodies can be derived or isolated from patients with SLE. In some embodiments, the anti-DNA antibodies are monoclonal antibodies, or fragments or variants thereof.

Exemplary antibodies that can be used include whole immunoglobulin (i.e., an intact antibody) of any class, fragments thereof, and synthetic proteins containing at least the antigen binding variable domain of an antibody. The variable domains differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each include four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen binding site of antibodies. Therefore, the antibodies can contain the components of the CDRs necessary to penetrate cells, maintain DNA binding and/or interfere with DNA repair.

Also disclosed are variants and fragments of antibodies which have bioactivity. The fragments, whether attached to other sequences or not, include insertions, deletions, substitutions, or other selected modifications of particular regions or specific amino acids residues, provided the activity of the fragment is not significantly altered or impaired compared to the non-modified antibody or antibody fragment.

Techniques can also be adapted for the production of single-chain antibodies specific to an antigenic protein. Methods for the production of single-chain antibodies are well known to those of skill in the art. A single chain antibody can be created by fusing together the variable domains of the heavy and light chains using a short peptide linker, thereby reconstituting an antigen binding site on a single molecule. Single-chain antibody variable fragments (scFvs) in which the C-terminus of one variable domain is tethered to the N-terminus of the other variable domain via a 15 to 25 amino acid peptide or linker have been developed without significantly disrupting antigen binding or specificity of the binding. The linker is chosen to permit the heavy chain and light chain to bind together in their proper conformational orientation.

The anti-DNA antibodies can be modified to improve their therapeutic potential. For example, in some embodiments, the anti-DNA antibody is conjugated to another antibody specific for a second therapeutic target, for example, on or near a cancer cell or in a tumor microenvironment. For example, the anti-DNA antibody can be a fusion protein containing single chain variable fragment that binds DNA or nucleosomes and a single chain variable fragment of a monoclonal antibody that specifically binds the second therapeutic target. In other embodiments, the anti-DNA antibody is a bispecific antibody having a first heavy chain and a first light chain from an anti-DNA antibody and a second heavy chain and a second light chain from a monoclonal antibody that specifically binds the second therapeutic target.

Divalent single-chain variable fragments (di-scFvs) can be engineered by linking two scFvs. This can be done by producing a single peptide chain with two VH and two VL regions, yielding tandem scFvs. ScFvs can also be designed with linker peptides that are too short for the two variable regions to fold together (about five amino acids), forcing scFvs to dimerize. This type is known as diabodies. Diabodies have been shown to have dissociation constants up to 40-fold lower than corresponding scFvs, meaning that they have a much higher affinity to their target. Still shorter linkers (one or two amino acids) lead to the formation of trimers (triabodies or tribodies). Tetrabodies have also been produced. They exhibit an even higher affinity to their targets than diabodies.

The antibody can be a humanized or chimeric antibody, or a fragment, variant, or fusion protein thereof. Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain. Antibody humanization techniques generally involve the use of recombinant DNA technology to manipulate the DNA sequence encoding one or more polypeptide chains of an antibody molecule.

In some embodiments, the antibody is modified to alter its half-life. In some embodiments, it is desirable to increase the half-life of the antibody so that it is present in the circulation or at the site of treatment for longer periods of time. In other embodiments, the half-life of the anti-DNA antibody is decreased to reduce potential side effects. Antibody fragments are expected to have a shorter half-life than full size antibodies. Other methods of altering half-life are known and can be used in the described methods. For example, antibodies can be engineered with Fc variants that extend half-life, e.g., using Xtend™ antibody half-life prolongation technology (Xencor, Monrovia, CA).

In some embodiments, the antibody is conjugated to a cell-penetrating moiety, such as a cell-penetrating peptide, to facilitate entry into the cell and transport to the nucleus. Examples of cell-penetrating peptides include, but are not limited to, Polyarginine (e.g., R₉), Antennapedia sequences, TAT, HIV-Tat, Penetratin, Antp-3A (Antp mutant), Buforin II, Transportan, MAP (model amphipathic peptide), K-FGF, Ku70, Prion, pVEC, Pep-1, SynBl, Pep-7, HN-1, BGSC (Bis-Guanidinium-Spermidine-Cholesterol, and BGTC (Bis-Guanidinium-Tren-Cholesterol). In other embodiments, the antibody is modified using TransMabs™ technology (InNexus Biotech., Inc., Vancouver, BC).

In some embodiments, the anti-DNA antibody is 3E10, 5C6, or a variant, functional fragment, or fusion protein derived therefrom. For example, the anti-DNA antibody can have a V_{H} having an amino acid sequence as shown below and a V_{L} having an amino acid sequence as shown below. Exemplary variants include antibodies having a V_{H} including an amino acid sequence at least 90% identical to the amino acid sequence shown below and a V_{L} including an amino acid sequence at least 90% identical to an amino acid sequence shown below. Other exemplary variants include antibodies having a V_{H} including an amino acid sequence at least 95%, at least 98%, at least 99% identical to the amino acid sequence shown below and a V_{L} including an amino acid sequence at least 95%, at least 98%, at least 99% identical to the sequence as shown below.

1. Exemplary Binding Proteins

A panel of hybridomas, including the 3E10 and 5C6 hybridomas was previously generated from the MRLmpj/lpr lupus mouse model and DNA binding activity was evaluated (Zack, et al., J. Immunol. 154:1987-1994 (1995); Gu, et al., J. Immunol., 161:6999-7006 (1998)). Murine 3E10 can refer to the monoclonal antibody produced by ATCC Accession No. PTA 2439 hybridoma. 5C6 can refer to the monoclonal anti-DNA antibody with nucleolytic activity produced by a hybridoma from MRL/lpr lupus mouse model as described in Noble et al., 2014, Sci Rep 4:5958 doi: 10.1038/srep05958.

Thus in some embodiments, the cell-penetrating antibody is 3E10 or 5C6 antibody or a variant, fragment, and fusion protein thereof, or a humanized form thereof. Each can be used, alone or in combination, in the disclosed methods.

a. 3E10

In the early 1990s a murine lupus anti-DNA antibody, 3E10, was tested in experimental vaccine therapy for SLE. These efforts were aimed at developing anti-idiotype antibodies that would specifically bind anti-DNA antibody in SLE patients. However, 3E10 was serendipitously found to penetrate into living cells and nuclei without causing any observed cytotoxicity (Weisbart R H, et al. J Immunol. 1990 144(7): 2653-2658; Zack D J, et al. J Immunol. 1996 157(5): 2082-2088). Studies on 3E10 in SLE vaccine therapy were then supplanted by efforts focused on development of 3E10 as a molecular delivery vehicle for transport of therapeutic molecules into cells and nuclei. 3E10 preferentially binds DNA single-strand tails, inhibits key steps in DNA single-strand and double-strand break repair (Hansen, et al., Science Translational Medicine, 4:157ra142 (2012)). The 3E10 antibody and its single chain variable fragment which includes a D31N mutation in CDR1 of the V_{H} (3E10 (D31N) scFv) and di- and tri-valent fusions thereof penetrate into cells and nuclei and have proven capable of transporting therapeutic protein cargoes attached to the antibody either through chemical conjugation or recombinant fusion. Protein cargoes delivered to cells by 3E10 or 3E10 (D31N) scFv include catalase, p53, and Hsp70 (Weisbart R H, et al. J Immunol. 2000 164: 6020-6026; Hansen J E, et al. Cancer Res. 2007 Feb. 15; 67(4): 1769-74; Hansen J E, et al. Brain Res. 2006 May 9; 1088(1): 187-96). 3E10 (D31N) scFv effectively mediated delivery of Hsp70 to neurons in vivo and this resulted in decreased cerebral infarct volumes and improved neurologic function in a rat stroke model (Zhan X, et al. Stroke. 2010 41(3): 538-43).

3E10 and 3E10 (D31N) scFv and di- and tri-valent fusions thereof, without being conjugated to any therapeutic protein, enhance cancer cell radiosensitivity and chemosensitivity and that this effect is potentiated in cells deficient in DNA repair. Moreover, 3E10 and 3E10 scFv and di- and tri-valent fusions thereof are selectively lethal to cancer cells deficient in DNA repair even in the absence of radiation or chemotherapy. The Food and Drug Administration (FDA) has established a pathway for the development of monoclonal antibodies into human therapies, and 3E10 has already been approved by the FDA for use in a Phase I human clinical trial designed to test the efficacy of 3E10 in experimental vaccine therapy for SLE (Spertini F, et al. J Rheumatol. 1999 26(12): 2602-8).

Experiments indicate that 3E10 (D31N) scFv penetrates cell nuclei by first binding to extracellular DNA or its degradation products and then following them into cell nuclei through the ENT2 nucleoside salvage pathway (Weisbart, Scientific Reports, 5: Article number: 12022 (2015) doi:10.1038/srep12022). When administered to mice and rats 3E10 is preferentially attracted to tissues in which extracellular DNA is enriched, including tumors, regions of ischemic brain in stroke models, and skeletal muscle subject to contractile injury (Weisbart, et al., Sci Rep., 5:12022 (2015), Hansen, et al., J Biol Chem, 282(29):20790-20793 (2007), Weisbart, et al., Mol Immunol, 39(13):783-789 (2003), Zhan, et al., Stroke: A Journal of Cerebral Circulation, 41(3):538-543 (2010)). Thus the presence of extracellular DNA enhances the nuclear uptake of 3E10 (D31N) scFv. Furthermore, 3E10 (D31N) scFv preferentially localizes into tumor cell nuclei in vivo, likely due to increased DNA in the local environment released from ischemic and necrotic regions of tumor.

b. 5C6

5C6 induces γH2AX in BRCA2⁽⁻⁾ but not BRCA2⁽⁺⁾ cells and selectively suppresses the growth of the BRCA2⁽⁻⁾ cells. Mechanistically, 5C6 appears to induce senescence in the BRCA2⁽⁻⁾ cells. Senescence is a well-known response to DNA damage, and DNA damaging agents, including many chemotherapeutics, induce senescence after prolonged exposure (Sliwinska, et al., Mech. Ageing Dev., 130:24-32 (2009); to Poele, et al., Cancer Res. 62:1876-1883 (2002); Achuthan, et al., J. Biol. Chem., 286:37813-37829 (2011)). These observations establish that 5C6 penetrates cell nuclei and damages DNA, and that cells with preexisting defects in DNA repair due to BRCA2 deficiency are more sensitive to this damage than cells with intact DNA repair. See U.S. Published Application No. 2015/0376279.

2. Fragments and Fusion Proteins

In some embodiments, the antibody is one or more antigen binding antibody fragments and/or antigen binding fusion proteins of the antibody 3E10 or 5C6, or a variant thereof. The antigen binding molecules typically bind to the epitope of 3E10 or 5C6, and can, for example, maintain a function or activity of the full antibody.

Exemplary fragments and fusions include, but are not limited to, single chain antibodies, single chain variable fragments (scFv), di-scFv, tri-scFv, diabody, triabody, tetrabody, disulfide-linked Fvs (sdFv), Fab′, F(ab′)₂, Fv, and single domain antibody fragments (sdAb).

In some embodiments, the antibody includes two or more scFv. For example, the targeting moiety can be a scFv or a di-scFv. In some embodiments, each scFv can include one, two, or all three complementarity determining regions (CDRs) of the heavy chain variable region (V_{L}) of 3E10 or 5C6, or a variant thereof. The scFv can include one, two, or all three CDRs of the light chain variable region (V_{L}) of 3E10 or 5C6, or a variant thereof. The molecule can include the heavy chain variable region and/or light chain variable region of 3E10 or 5C6, or a variant thereof.

A single chain variable fragment can be created by fusing together the variable domains of the heavy and light chains using a short peptide linker, thereby reconstituting an antigen binding site on a single molecule. Single-chain antibody variable fragments (scFvs) in which the C-terminus of one variable domain is tethered to the N-terminus of the other variable domain via a linker have been developed without significantly disrupting antigen binding or specificity of the binding. The linker is chosen to permit the heavy chain and light chain to bind together in their proper conformational orientation. The linker is usually rich in glycine for flexibility, and typically also includes serine or threonine for solubility. The linker can link, for example, the N-terminus of the V_{H} with the C-terminus of the V_{L}, or vice versa. scFv can also be created directly from subcloned heavy and light chains derived from a hybridoma. In some embodiments, the scFv retains, or improves or increases the specificity of the original immunoglobulin, while removing of the constant regions and introducing the linker.

Exemplary molecules that include two or more single chain variable fragments (scFv) including the light chain variable region (V_{L}) of 3E10 or 5C6, or a variant thereof, and the heavy chain variable region (V_{H}) of 3E10 or 5C6, or a variant thereof of the antibody 3E10 or 5C6 include, but are not limited to, divalent-scFv (di-scFv), trivalent-scFv (tri-scFv), multivalent-scFv (multi-scFv), diabodies, triabodies, tetrabodies, etc., of scFvs.

Divalent single chain variable fragments can be engineered by linking two scFvs. This can be done by producing a single peptide chain with two V_{H} and two V_{L} regions, yielding a di-scFvs referred to as a tandem di-scFv. ScFvs can also be designed with linker peptides that are too short for the two variable regions to fold together (about five amino acids), forcing scFvs to dimerize and form a divalent single chain variable fragment referred to as a diabody. Diabodies have been shown to have dissociation constants up to 40-fold lower than corresponding scFvs, indicating that they have a much higher affinity to their target. Even shorter linkers (one or two amino acids) lead to the formation of trimers (triabodies or tribodies). Tetrabodies have also been produced and have been shown to exhibit an even higher affinity to their targets than diabodies.

The disclosed antibodies include antigen binding antibody fragments and fusion proteins of 3E10 or 5C6 and variants thereof that can bind to the same epitope as the parent antibody 3E10 or 5C6. In some embodiments, the antigen binding molecule is a di-, tri-, or multivalent scFv. Although the antigen binding antibody fragment or fusion protein of the antigen binding molecule can include additional antibody domains (e.g., constant domains, hinge domains, etc.), in some embodiments it does not. For example, 3E10 binds DNA and impairs DNA repair, which is synthetically lethal to DNA repair-deficient cells. This function is independent of any 3E10 constant regions. By contrast, non-penetrating antibodies such as cetuximab that target extracellular receptors depend in part on Fc-mediated activation of ADCC and complement to exert an effect on tumors Elimination of the Fc from non-penetrating antibodies could therefore diminish the magnitude of their effect on tumors, but Fc is not required for 3E10 to have an effect on cancer cells. Therefore, 3E10 fragments or fusions that lack an Fc region should be unable to activate ADCC and complement and therefore carry a lower risk of nonspecific side effects.

a. Single Chain Variable Fragments

The single chain variable fragments disclosed herein can include antigen binding fragments of 3E10 or 5C6, or a variant thereof. The monoclonal antibody 3E10 and active fragments and exemplary variants thereof that are transported in vivo to the nucleus of mammalian cells without cytotoxic effect are discussed in U.S. Pat. Nos. 4,812,397 and 7,189,396, and U.S. Published Application No. 2014/0050723. Other 3E10 antibody compositions, including fragments and fusions thereof, suitable for use with the disclosed compositions and methods are discussed in, for example, WO 2012/135831, WO 2016/033321, WO 2015/106290, and WO 2016/033324. 5C6 is described in U.S. Published Application No. 2015/0376279. Sequences for single and two or more linked single chain variable fragments of 3E10 are provided in WO 2017/218825 and WO 2016/033321. Exemplary 3E10 humanized sequences are discussed in WO 2015/106290 and WO 2016/033324.

An scFv includes a light chain variable region (V_{L}) and a heavy chain variable region (V_{H}) joined by a linker. For example, the linker can include in excess of 12 amino acid residues with (Gly₄Ser)₃ (SEQ ID NO:26) being one of the more favored linkers for a scFv. The scFv can be a disulfide stabilized Fv (or diFv or dsFv), in which a single cysteine residue is introduced into a FR of V_{H} and a FR of V_{L} and the cysteine residues linked by a disulfide bond to yield a stable Fv. The scFv can be a dimeric scFv (di-scFV), i.e., a protein including two scFv molecules linked by a non-covalent or covalent linkage, e.g., by a leucine zipper domain (e.g., derived from Fos or Jun) or trimeric scFV (tri-scFv). In another example, two scFv's are linked by a peptide linker of sufficient length to permit both scFv's to form and to bind to an antigen, e.g., as described in U.S. Published Application No. 2006/0263367.

The variable domains differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each include four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen binding site of antibodies.

The fragments and fusions of antibodies disclosed herein can have bioactivity. For example, the fragments and fusions, whether attached to other sequences or not, can include insertions, deletions, substitutions, or other selected modifications of particular regions or specific amino acids residues. In some embodiments, the activity of the fragment or fusion is not significantly reduced or impaired compared to the nonmodified antibody or antibody fragment.

b. Sequences

i. 3E10 Light Chain Variable Region

An amino acid sequence for the light chain variable region of 3E10 is:

DIVLTQSPASLAVSLGQRATISCRASKSVSTSSYSYMHWYQQKPGQP PKLLIKYASYLESGVPARFSGSGSGTDFTLNIHPVEEEDAATYYCQHS REFPWTFGGGTKLEIK (SEQ ID NO:1). The complementarity determining regions (CDRs) as defined by Kabat are shown with underlining. Other 3E10 light chain sequences are known in the art. See, for example, Zack, et al., J. Immunol., 15; 154(4):1987-94 (1995); GenBank: L16981.1—Mouse Ig rearranged L-chain gene, partial cds; GenBank: AAA65681.1—immunoglobulin light chain, partial [Mus musculus]).

An amino acid sequence for the light chain variable region of 3E10 can also be:

(SEQ ID NO: 2)
DIVLTQSPASLAVSLGQRATISCRASKSVSTSSYSYMHWYQQKPGQP
PKLLIKYASYLESGVPARFSGSGSGTDFHLNIHPVEEEDAATYYCQHS
REFPWTFGGGTKLELK.

The complementarity determining regions (CDRs) as defined by Kabat are shown with underlining, including CDR L1.1:

(SEQ ID NO: 34)
RASKSVSTSSYSYMH;
CDR L2.1:
(SEQ ID NO: 36)
YASYLES;
CDR L3.1:
(SEQ ID NO: 37)
QHSREFPWT.
Variants of Kabat CDR L1.1 include
(SEQ ID NO: 91)
RASKSVSTSSYSYLA
and
(SEQ ID NO: 35)
RASKTVSTSSYSYMH.
A variant of Kabat CDR L2.1 is
(SEQ ID NO: 90)
YASYLQS.

Additionally, or alternatively, the heavy chain complementarity determining regions (CDRs) can be defined according to the IMGT system. The complementarity determining regions (CDRs) as defined by the IMGT system include CDR L1.2 KSVSTSSYSY (SEQ ID NO:42); CDR L2.2: YAS (SEQ ID NO:44); CDR L3.2: QHSREFPWT (SEQ ID NO:37).

A variant of CDR L1.2 is
(SEQ ID NO: 43)
KTVSTSSYSY.

In some embodiments, the C-terminal end of sequence of SEQ ID NOS:1 or 2 further includes an arginine in the 3E10 light chain variable region.

ii. 3E10 Heavy Chain Variable Region

An amino acid sequence for the heavy chain variable region of 3E10 is:

### EVQLVESGGGLVKPGGSRKLSCAASGFTFSDYGMHWVRQAPEKGLE WVAYISSGSSTIYYADTVKGRFTISRDNAKNTLFLQMTSLRSEDTAM YYCARRGLLLDYWGQGTTLTVSS

(SEQ ID NO:6; Zack, et al., Immunology and Cell Biology, 72:513-520 (1994); GenBank: L16981.1—Mouse Ig rearranged L-chain gene, partial cds; and GenBank: AAA65679.1—immunoglobulin heavy chain, partial [Mus musculus]). The complementarity determining regions (CDRs) as defined by Kabat are shown with underlining

An amino acid sequence for a preferred variant of the heavy chain variable region of 3E10 is:

EVQLVESGGGLVKPGGSRKLSCAASGFTFSNYGMHWVRQAPEKGLE WVAYISSGSSTIYYADTVKGRFTISRDNAKNTLFLQMTSLRSEDTAM YYCARRGLLLDYWGQGTTLTVSS (SEQ ID NO:7). The complementarity determining regions (CDRs) as defined by Kabat are shown with underlining

In some embodiments, the C-terminal serine of SEQ ID NOS:6 or 7 is absent or substituted, with, for example, an alanine, in 3E10 heavy chain variable region.

Amino acid position 31 of the heavy chain variable region of 3E10 has been determined to be influential in the ability of the antibody and fragments thereof to penetrate nuclei and bind to DNA. For example, D31N mutation (bolded and italicized in SEQ ID NOS:1 and 2) in CDR1 penetrates nuclei and binds DNA with much greater efficiency than the original antibody (Zack, et al., Immunology and Cell Biology, 72:513-520 (1994), Weisbart, et al., J. Autoimmun., 11, 539-546 (1998); Weisbart, Int. J. Oncol., 25, 1867-1873 (2004)).

The complementarity determining regions (CDRs) as defined by Kabat are shown with underlining, including CDR H1.1 (original sequence):

(SEQ ID NO: 8)
DYGMH;
CDR H1.2 (with D31N mutation):
(SEQ ID NO: 30)
NYGMH;
CDR H2.1:
(SEQ ID NO: 10)
YISSGSSTIYYADTVKG;
CDR H3.1:
(SEQ ID NO: 33)
RGLLLDY.
Variants of Kabat CDR H2.1 include
(SEQ ID NO: 32)
YISSGSSTIYYADSVKG
and
(SEQ ID NO: 31)
YISSSSSTIYYADSVKG.

Additionally, or alternatively, the heavy chain complementarity determining regions (CDRs) can be defined according to the IMGT system. The complementarity determining regions (CDRs) as defined by the IMGT system include CDR H1.3 (original sequence): GFTFSDYG (SEQ ID NO:89); CDR H1.4 (with D31N mutation): GFTFSNYG (SEQ ID NO:38); CDR H2.2: ISSGSSTI (SEQ ID NO:40); CDR H3.2: ARRGLLLDY (SEQ ID NO:41).

A variant of CDR H2.2 is
(SEQ ID NO: 39)
ISSSSSTI.

In addition to 3E10 and its fragments described above, additional anti-DNA antibodies may be used in the disclosed compositions and methods. These include the nuclear-penetrating anti-DNA antibody 5C6 as specified below.

iii. 5C6 Light Chain Variable Region

An amino acid sequence for the kappa light chain variable region (V_{L}) of mAb 5C6 is:

(SEQ ID NO: 12)
DIVLTQSPASLAAVSLGERATISYRASKSVSTSGYSYMHWNQQKPGQ
APRLLIYLVSNLESGVPARFSGSGSGTDFTLNIHPVEEEDAATYYCQH
IRELDTFFGGGTKLEIK.

The complementarity determining regions (CDRs) are shown with underlining, including CDR L1: RASKSVSTSGYSYMH (SEQ ID NO:13); CDR L2: LVSNLES (SEQ ID NO:14); CDR L3: QHIRELDTF (SEQ ID NO:15).

iv. 5C6 Heavy Chain Variable Region

An amino acid sequence for the heavy chain variable region (V_{H}) of mAb 5C6 is:

(SEQ ID NO: 16)
QLKLVESGGGLVKPGGSLKLSCAASGFTFSSYTMSWVRQTPAKRLE
WVATISSGGGSTYYPDSVKGRFTISRDNARNTLYLQMSSLRSEDTAM
YYCARRAYSKRGAMDYWGQGTSVTVSS.

The complementarity determining regions (CDRs) are shown with underlining, including CDR H1: SYTMS (SEQ ID NO:17); CDR H2:

(SEQ ID NO: 18)
TISSGGGSTYYPDSVKG;
CDR H3:
(SEQ ID NO: 19)
RAYSKRGAMDY.

c. Linkers

The term “linker” as used herein includes, without limitation, peptide linkers. The peptide linker can be any size provided it does not interfere with the binding of the epitope by the variable regions. In some embodiments, the linker includes one or more glycine and/or serine amino acid residues. Monovalent single-chain antibody variable fragments (scFvs) in which the C-terminus of one variable domain are typically tethered to the N-terminus of the other variable domain via a 15 to 25 amino acid peptide or linker. The linker is chosen to permit the heavy chain and light chain to bind together in their proper conformational orientation. Linkers in diabodies, triabodies, etc., typically include a shorter linker than that of a monovalent scFv as discussed above. Di-, tri-, and other multivalent scFvs typically include three or more linkers. The linkers can be the same, or different, in length and/or amino acid composition. Therefore, the number of linkers, composition of the linker(s), and length of the linker(s) can be determined based on the desired valency of the scFv as is known in the art. The linker(s) can allow for or drive formation of a di-, tri-, and other multivalent scFv.

For example, a linker can include 4-8 amino acids. In a particular embodiment, a linker includes the amino acid sequence GQSSRSS (SEQ ID NO:20). In another embodiment, a linker includes 15-20 amino acids, for example, 18 amino acids. In a particular embodiment, the linker includes the amino acid sequence GQSSRSSSGGGSSGGGGS (SEQ ID NO:21). Other flexible linkers include, but are not limited to, the amino acid sequences Gly-Ser, Gly-Ser-Gly-Ser (SEQ ID NO:22), Ala-Ser, Gly-Gly-Gly-Ser (SEQ ID NO:23), (Gly₄-Ser)₂ (SEQ ID NO:24) and (Gly₄-Ser)₄ (SEQ ID NO:25), and (Gly-Gly-Gly-Gly-Ser)₃ (SEQ ID NO:26).

d. Variants

The antibody can be composed of or include an antibody fragment or fusion protein including an amino acid sequence of a variable heavy chain and/or variable light chain that is at least 45%, at least 50%, at least 55%, at least 60%, at least 65%, at least 70%, at least 75%, at least 80%, at least 85%, at least 90%, at least 95%, or at least 99% identical to the amino acid sequence of the variable heavy chain and/or light chain of 3E10 or 5C6 or a humanized form thereof, including to any of the exemplary sequences provided herein. In some embodiments, the antibody binds to the epitope of 3E10 or 5C6, is selectively lethal to or selectively increases the radiosensitivity and/or chemosensitivity of cells deficient in DNA repair, or a combination thereof.

The antibody can be composed of or include an antibody fragment or fusion protein that includes a CDR that is at least 45%, at least 50%, at least 55%, at least 60%, at least 65%, at least 70%, at least 75%, at least 80%, at least 85%, at least 90%, at least 95%, or at least 99% identical to the amino acid sequence of a CDR of the variable heavy chain and/or light chain of 3E10 or 5C6 and/or a humanized form thereof, including to any of the exemplary sequences provided herein. In some embodiments, the antibody binds to the epitope of 3E10 or 5C6, is selectively lethal to or selectively increases the radiosensitivity and/or chemosensitivity of cells deficient in DNA repair, or a combination thereof.

The determination of percent identity of two amino acid sequences can be determined by BLAST protein comparison. In some embodiments, scFv includes one, two, three, four, five, or all six of the CDRs of the above-described preferred variable domains and which binds to the epitope of 3E10 or 5C6, is selectively lethal to or selectively increases the radiosensitivity and/or chemosensitivity of cells deficient in DNA repair, or a combination thereof.

Predicted complementarity determining regions (CDRs) of the light chain variable sequence for 3E10 or 5C6 are provided above. See also GenBank: AAA65681.1—immunoglobulin light chain, partial [Mus musculus]. Predicted complementarity determining regions (CDRs) of the heavy chain variable sequence for 3E10 and 5C6 are provide above. See, for example, Zack, et al., Immunology and Cell Biology, 72:513-520 (1994) and GenBank Accession number AAA65679.1. Exemplary humanized 3E10 sequences and scFv are provided below.

e. Exemplary Humanized anti-DNA Binding Proteins

Exemplary anti-DNA binding proteins, and exemplary human IgG1 hinge and constant regions are disclosed in International Patent Application PCT/US2018/042532, and International Patent Application PCT/US2018/042534, and provided below. Cell-penetrating antibodies for use in the disclosed combination therapies include those having the exemplary humanized CDR, the exemplary humanized heavy chain variable regions, and/or the exemplary humanized light chain variable regions, and fragments and variants thereof.

The binding proteins and antibodies herein can have, for example, any combination of light and heavy chain CDR1-3 sequences provided herein. The binding protein and antibodies herein can have, for example any combination of light and heavy chain region sequences provided herein.

In some embodiments, the anti-DNA binding proteins include a V_{H} having a CDR1 as shown in SEQ ID NO:30, a CDR2 as shown in SEQ ID NO:31 or SEQ ID NO:32 and a CDR3 as shown in SEQ ID NO:33 and a V_{L} having a CDR1 as shown in SEQ ID NO:34 or SEQ ID NO:35, a CDR2 as shown in SEQ ID NO:36 and a CDR3 as shown in SEQ ID NO:37. For example, an anti-DNA binding protein can include a V_{H} having a CDR1 as shown in SEQ ID NO:30, a CDR2 as shown in SEQ ID NO:31 and a CDR3 as shown in SEQ ID NO:33 and a V_{L} having a CDR1 as shown in SEQ ID NO:34, a CDR2 as shown in SEQ ID NO:36 and a CDR3 as shown in SEQ ID NO:37. In another embodiment, an anti-DNA binding protein can include a V_{H} having a CDR1 as shown in SEQ ID NO:30, a CDR2 as shown in SEQ ID NO:31 and a CDR3 as shown in SEQ ID NO:33 and a V_{L} having a CDR1 as shown in SEQ ID NO:35, a CDR2 as shown in SEQ ID NO:36 and a CDR3 as shown in SEQ ID NO:37. In another embodiment, an anti-DNA binding protein can include a V_{H} having a CDR1 as shown in SEQ ID NO:30, a CDR2 as shown in SEQ ID NO:32 and a CDR3 as shown in SEQ ID NO:33 and a V_{L} having a CDR1 as shown in SEQ ID NO:34, a CDR2 as shown in SEQ ID NO:36 and a CDR3 as shown in SEQ ID NO:37. In another embodiment, an anti-DNA binding protein can include a V_{H} having a CDR1 as shown in SEQ ID NO:30, a CDR2 as shown in SEQ ID NO:32 and a CDR3 as shown in SEQ ID NO:33 and a V_{L} having a CDR1 as shown in SEQ ID NO:35, a CDR2 as shown in SEQ ID NO:36 and a CDR3 as shown in SEQ ID NO:37.

Above exemplified binding proteins may also have CDRs assigned using the IMGT system. Appropriate sequences from this system are referenced below.

In another embodiment, the anti-DNA binding proteins include a V_{H} including a sequence at least 95% identical to the sequence as shown in any one of SEQ ID NOs:9, 11, or 45 to 52 and a V_{L} including a sequence at least 95% identical to the sequence as shown in any one of SEQ ID NOs:3 to 5, or 53 to 58. For example, an anti-DNA binding protein can include a V_{H} including a sequence at least 95% identical to the sequence as shown in SEQ ID NO:47 and a V_{L} including a sequence at least 95% identical to the sequence as shown in SEQ ID NO:54. In another embodiment, an anti-DNA binding protein can include a V_{H} including a sequence at least 95% identical to the sequence as shown in SEQ ID NO:52 and a V_{L} including a sequence at least 95% identical to the sequence as shown in SEQ ID NO:56. In these embodiments, the V_{H} and/or V_{L} can be at least 96%, at least 97%, at least 98% or at least 99% identical to the recited SEQ ID NO.

In some embodiments, the anti-DNA binding proteins include a V_{H} including a sequence as shown in any one of SEQ ID NOs:9, 11, or 45 to 52 and a V_{L} including a sequence as shown in any one of SEQ ID NOs:3 to 5 or 53 to 58. For example, an anti-DNA binding protein can include a V_{H} including a sequence as shown in SEQ ID NO:47 and a V_{L} including a sequence as shown in SEQ ID NO:54. In another embodiment, an anti-DNA binding protein can include a V_{H} including a sequence as shown in SEQ ID NO:52 and a V_{L} including a sequence as shown in SEQ ID NO:56.

In some embodiments, the anti-DNA binding protein can be a cell-penetrating anti-DNA Fv fragment having an antigen binding domain, wherein the antigen binding domain binds to or specifically binds to DNA. For example, the Fv can bind the same epitope as a binding protein having a V_{H} including an amino acid sequence as shown in SEQ ID NO:7 and a V_{L} including an amino acid sequence as shown in SEQ ID NO:2. In another embodiment, the Fv can bind the same epitope as a di-scFv having an amino acid sequence as shown in SEQ ID NO:28. In some embodiments, the Fv includes a V_{H} having a CDR1 as shown in SEQ ID NO:30, a CDR2 as shown in SEQ ID NO:31 or SEQ ID NO:32 and a CDR3 as shown in SEQ ID NO:33 and a V_{L} having a CDR1 as shown in SEQ ID NO:34 or SEQ ID NO:35, a CDR2 as shown in SEQ ID NO:36 and a CDR3 as shown in SEQ ID NO:37. For example, an Fv can include a V_{H} having a CDR1 as shown in SEQ ID NO:30, a CDR2 as shown in SEQ ID NO:31 and a CDR3 as shown in SEQ ID NO:33 and a V_{L} having a CDR1 as shown in SEQ ID NO:34, a CDR2 as shown in SEQ ID NO:36 and a CDR3 as shown in SEQ ID NO:37. In another embodiment, an Fv can include a V_{H} having a CDR1 as shown in SEQ ID NO:30, a CDR2 as shown in SEQ ID NO:31 and a CDR3 as shown in SEQ ID NO:33 and a V_{L} having a CDR1 as shown in SEQ ID NO:35, a CDR2 as shown in SEQ ID NO:36 and a CDR3 as shown in SEQ ID NO:37. In another embodiment, an Fv can include a V_{H} having a CDR1 as shown in SEQ ID NO:30, a CDR2 as shown in SEQ ID NO:32 and a CDR3 as shown in SEQ ID NO:33 and a V_{L} having a CDR1 as shown in SEQ ID NO:34, a CDR2 as shown in SEQ ID NO:36 and a CDR3 as shown in SEQ ID NO:37. In another embodiment, an Fv can include a V_{H} having a CDR1 as shown in SEQ ID NO:30, a CDR2 as shown in SEQ ID NO:32 and a CDR3 as shown in SEQ ID NO:33 and a V_{L} having a CDR1 as shown in SEQ ID NO:35, a CDR2 as shown in SEQ ID NO:36 and a CDR3 as shown in SEQ ID NO:37.

Above exemplified Fv may also have CDRs assigned using the IMGT system. Appropriate sequences from this system are referenced below.

In another embodiment, the Fv includes a V_{H} including a sequence at least 95% identical to the sequence as shown in any one of SEQ ID NOs:9, 1, or 45 to 52 and a V_{L} including a sequence at least 95% identical to the sequence as shown in any one of SEQ ID NOs:3 to 5, or 53 to 58. For example, an Fv can include a V_{H} including a sequence at least 95% identical to the sequence as shown in SEQ ID NO:47 and a V_{L} including a sequence at least 95% identical to the sequence as shown in SEQ ID NO:54. In another embodiment, an Fv can include a V_{H} including a sequence at least 95% identical to the sequence as shown in SEQ ID NO:50 and a V_{L} including a sequence at least 95% identical to the sequence as shown in SEQ ID NO:56. In another embodiment, an Fv can include a V_{H} including a sequence at least 95% identical to the sequence as shown in SEQ ID NO:52 and a V_{L} including a sequence at least 95% identical to the sequence as shown in SEQ ID NO:56. In these embodiments, the V_{H} and/or V_{L} can be at least 96%, at least 97%, at least 98% or at least 99% identical to the recited SEQ ID NO. In these embodiments, the Fv can have an above referenced combination of CDRs. For example, an Fv can include a V_{H} including a sequence at least 95% identical to the sequence as shown in SEQ ID NO:50 and a V_{L} including a sequence at least 95% identical to the sequence as shown in SEQ ID NO:56, wherein the V_{H} has a CDR1 as shown in SEQ ID NO:30, a CDR2 as shown in SEQ ID NO:32 and a CDR3 as shown in SEQ ID NO:33 and the V_{L} has a CDR1 as shown in SEQ ID NO:35, a CDR2 as shown in SEQ ID NO:36 and a CDR3 as shown in SEQ ID NO:37.

In another embodiment, the Fv includes a V_{H} including a sequence as shown in any one of SEQ ID NOs:9, 11, or 45 to 52 and a V_{L} including a sequence as shown in any one of SEQ ID NOs:3 to 5 or 53 to 58. For example, an Fv can include a V_{H} including a sequence as shown in SEQ ID NO:50 and a V_{L} including a sequence as shown in SEQ ID NO:56. In another embodiment, an Fv can include a V_{H} including a sequence as shown in SEQ ID NO:52 and a V_{L} including a sequence as shown in SEQ ID NO:56.

In some embodiments, the V_{H} and V_{L} of the Fv can be in a single polypeptide chain. In another embodiment, the Fv lacks an Fc region. For example, the Fv can be a single chain Fv fragment (scFv), a dimeric scFv (di-scFv), a trimeric scFv (tri-scFv). In some embodiments, the Fv is an scFv. In another embodiment, the Fv is a di-scFv. In another embodiment, the Fv is a tri-scFv.

In another embodiment, the scFv, di-scFv or tri-scFv can be linked to a constant region of an antibody, Fc or a heavy chain constant domain C_{H}2 and/or C_{H}3.

In some embodiments, the present disclosure encompasses a cell-penetrating di-scFv having an antigen binding domain, wherein the antigen binding domain binds to or specifically binds to DNA.

In some embodiments, a di-scFv according to the present disclosure includes an amino acid sequence at least 95% identical to the sequence as shown in any one of SEQ ID NOs:61 to 76. For example, the di-scFv includes an amino acid sequence at least 95% identical to the amino acid sequence shown in any one of SEQ ID NOs:61, 65, 70 or 72. In these embodiments, amino acid sequences can be at least 96%, at least 97%, at least 98% or at least 99% identical to the recited SEQ ID NO. In some embodiments, a di-scFv according to the present disclosure includes an amino acid sequence as shown in any one of SEQ ID NOs:61 to 76. For example, the di-scFv can include an amino acid sequence as shown in any one of SEQ ID NOs:61, 65, 70 or 72.

In another embodiment, the V_{H} and V_{L} of the binding protein are in a separate polypeptide chain. For example, the binding protein can be a diabody, triabody, tetrabody, Fab, F(ab′) 2. In another embodiment, the binding protein can be an Fv which includes a V_{H} and V_{L} in separate polypeptide chains. In these embodiments, the binding proteins may be linked to a constant region of an antibody, Fc or a heavy chain constant domain C_{H}2 and/or C_{H}3.

In another embodiment, the binding protein can be an intact antibody. Accordingly, in some embodiments, the present disclosure encompasses an antibody having an antigen binding domain, wherein the antigen binding domain binds to or specifically binds to DNA. For example, the antibody can bind the same epitope as a binding protein having a V_{H} including an amino acid sequence as shown in SEQ ID NO:7 and a V_{L} including an amino acid sequence as shown in SEQ ID NO:2. In another embodiment, the antibody can bind the same epitope as a di-scFv having an amino acid sequence as shown in SEQ ID NO:28.

In another embodiment, the antibody includes a V_{H} having a CDR1 as shown in SEQ ID NO:30, a CDR2 as shown in SEQ ID NO:31 or SEQ ID NO:32 and a CDR3 as shown in SEQ ID NO:33 and a V_{L} having a CDR1 as shown in SEQ ID NO:34 or SEQ ID NO:35, a CDR2 as shown in SEQ ID NO:36 and a CDR3 as shown in SEQ ID NO:37. For example, an antibody can include a V_{H} having a CDR1 as shown in SEQ ID NO:30, a CDR2 as shown in SEQ ID NO:31 and a CDR3 as shown in SEQ ID NO:33 and a V_{L} having a CDR1 as shown in SEQ ID NO:34, a CDR2 as shown in SEQ ID NO:36 and a CDR3 as shown in SEQ ID NO:37. In another embodiment, an antibody can include a V_{H} having a CDR1 as shown in SEQ ID NO:30, a CDR2 as shown in SEQ ID NO:31 and a CDR3 as shown in SEQ ID NO:33 and a V_{L} having a CDR1 as shown in SEQ ID NO:35, a CDR2 as shown in SEQ ID NO:36 and a CDR3 as shown in SEQ ID NO:37. In another embodiment, an antibody can include a V_{H} having a CDR1 as shown in SEQ ID NO:30, a CDR2 as shown in SEQ ID NO:32 and a CDR3 as shown in SEQ ID NO:33 and a V_{L} having a CDR1 as shown in SEQ ID NO:34, a CDR2 as shown in SEQ ID NO:36 and a CDR3 as shown in SEQ ID NO:37. In another embodiment, an antibody can include a V_{H} having a CDR1 as shown in SEQ ID NO:30, a CDR2 as shown in SEQ ID NO:32 and a CDR3 as shown in SEQ ID NO:33 and a V_{L} having a CDR1 as shown in SEQ ID NO:35, a CDR2 as shown in SEQ ID NO:36 and a CDR3 as shown in SEQ ID NO:37.

Above exemplified antibodies may also have CDRs assigned using the IMGT system. Appropriate sequences from this system are referenced below.

In another embodiment, the antibody includes a V_{H} including a sequence at least 95% identical to the sequence as shown in any one of SEQ ID NOs:9, 11, or 45 to 52 and a V_{L} including a sequence at least 95% identical to the sequence as shown in any one of SEQ ID NOs:3 to 5, or 53 to 58. For example, an antibody can include a V_{H} including a sequence at least 95% identical to the sequence as shown in SEQ ID NO:47 and a V_{L} including a sequence at least 95% identical to the sequence as shown in SEQ ID NO:54. In another embodiment, an antibody can include a V_{H} including a sequence at least 95% identical to the sequence as shown in SEQ ID NO:50 and a V_{L} including a sequence at least 95% identical to the sequence as shown in SEQ ID NO:56. In another embodiment, an antibody can include a V_{H} including a sequence at least 95% identical to the sequence as shown in SEQ ID NO:52 and a V_{L} including a sequence at least 95% identical to the sequence as shown in SEQ ID NO:56. In these embodiments, the V_{H} and/or V_{L} can be at least 96%, at least 97%, at least 98% or at least 99% identical to the recited SEQ ID NO. In these embodiments, the antibody can have an above referenced combination of CDRs. For example, an antibody can include a V_{H} including a sequence at least 95% identical to the sequence as shown in SEQ ID NO:50 and a V_{L} including a sequence at least 95% identical to the sequence as shown in SEQ ID NO:56, wherein the V_{H} has a CDR1 as shown in SEQ ID NO:30, a CDR2 as shown in SEQ ID NO:32 and a CDR3 as shown in SEQ ID NO:33 and the V_{L} has a CDR1 as shown in SEQ ID NO:35, a CDR2 as shown in SEQ ID NO:36 and a CDR3 as shown in SEQ ID NO:37.

In another embodiment, the antibody includes a V_{H} including a sequence as shown in any one of SEQ ID NOs:9, 11, or 45 to 52 and a V_{L} including a sequence as shown in any one of SEQ ID NOs:3 to 5, or 53 to 58. For example, an antibody can include a V_{H} including a sequence as shown in SEQ ID NO:47 and a V_{L} including a sequence as shown in SEQ ID NO:54. In another embodiment, an antibody can include a V_{H} including a sequence as shown in SEQ ID NO:50 and a V_{L} including a sequence as shown in SEQ ID NO:56. In another embodiment, an antibody can include a V_{H} including a sequence as shown in SEQ ID NO:52 and a V_{L} including a sequence as shown in SEQ ID NO:56.

In another embodiment, the antibody has an amino acid sequence shown in any one of SEQ ID NOs:77, 82 or 84 and an amino acid sequence shown in SEQ ID NO:87.

Exemplary sequences from anti-DNA binding protein sequences encompassed by the present disclosure follow:

Heavy Chain CDR1 KABAT
(SEQ ID NO: 30)
NYGMH
Heavy Chain CDR2 (variants 2-4, 6-8, 10-12) KABAT
(SEQ ID NO: 31)
YISSSSSTIYYADSVKG
Heavy Chain CDR2 (variants 13-19) KABAT
(SEQ ID NO: 32)
YISSGSSTIYYADSVKG
Heavy Chain CDR3 KABAT
(SEQ ID NO: 33)
RGLLLDY
Light Chain CDR1 (variants 2-4, 6-8, 10-12) KABAT
(SEQ ID NO: 34)
RASKSVSTSSYSYMH
Light Chain CDR1 (variants 13-19) KABAT
(SEQ ID NO: 35)
RASKTVSTSSYSYMH
Light Chain CDR2 KABAT
(SEQ ID NO: 36)
YASYLES
Light Chain CDR3 KABAT
(SEQ ID NO: 37)
QHSREFPWT
Heavy Chain CDR1 IMGT
(SEQ ID NO: 38)
GFTFSNYG
Heavy Chain CDR2 (variants 2-4, 6-8, 10-12) IMGT
(SEQ ID NO: 39)
ISSSSSTI
Heavy Chain CDR2 (variants 13-19) IMGT
(SEQ ID NO: 40)
ISSGSSTI
Heavy Chain CDR3 IMGT
(SEQ ID NO: 41)
ARRGLLLDY
Light Chain CDR1 (variants 2-4, 6-8, 10-12) IMGT
(SEQ ID NO: 42)
KSVSTSSYSY
Light Chain CDR1 (variants 13-19) IMGT
(SEQ ID NO: 43)
KTVSTSSYSY
Light Chain CDR2 IMGT
(SEQ ID NO: 44)
YAS
Light Chain CDR3 IMGT
(SEQ ID NO: 37)
QHSREFPWT
Heavy Chain variable region (variants 2, 6 and 10)
(SEQ ID NO: 46)
EVQLVESGGGLVQPGGSLRLSCAASGFTFSNYGMHWVRQAPGKGLE
WVSYISSSSSTIYYADSVKGRFTISRDNAKNSLYLQMNSLRAEDTAV
YYCARRGLLLDYWGQGTTVTVSS
Heavy Chain variable region (variants 3, 7 and 11)
(SEQ ID NO: 47)
EVQLVESGGGVVQPGGSLRLSCAASGFTFSNYGMHWVRQAPEKGLE
WVSYISSSSSTIYYADSVKGRFTISRDNSKNTLYLQMNSLRAEDTAVY
YCARRGLLLDYWGQGTTVTVSS
Heavy Chain variable region (variants 4, 8 and 12)
(SEQ ID NO: 48)
EVQLVESGGGDVKPGGSLRLSCAASGFTFSNYGMHWVRQAPEKGLE
WVSYISSSSSTIYYADSVKGRFTISRDNSKNTLYLQMNSLRAEDTAVY
YCARRGLLLDYWGQGTTVTVSS
Heavy Chain variable region (variants 13, 16
and 19)
(SEQ ID NO: 50)
EVQLVESGGGLVQPGGSLRLSCAASGFTFSNYGMHWVRQAPGKGLE
WVSYISSGSSTIYYADSVKGRFTISRDNAKNSLYLQMNSLRAEDTAV
YYCARRGLLLDYWGQGTTVTVSS
Heavy Chain variable region (variants 14 and 17)
(SEQ ID NO: 51)
EVQLVESGGGVVQPGGSLRLSCAASGFTFSNYGMHWVRQAPEKGLE
WVSYISSGSSTIYYADSVKGRFTISRDNSKNTLYLQMNSLRAEDTAV
YYCARRGLLLDYWGQGTTVTVSS
Heavy Chain variable region (variants 15 and 18)
(SEQ ID NO: 52)
EVQLVESGGGDVKPGGSLRLSCAASGFTFSNYGMHWVRQAPEKGLE
WVSYISSGSSTIYYADSVKGRFTISRDNSKNTLYLQMNSLRAEDTAV
YYCARRGLLLDYWGQGTTVTVSS
Heavy Chain variable region (hVH1, WO 2016/033324)
(SEQ ID NO: 9)
EVQLVQSGGGLIQPGGSLRLSCAASGFTFSNYGMHWVRQAPGKGLE
WVSYISSGSSTIYYADSVKGRFTISRDNSKNTLYLQMNSLRAEDTAV
YYCARRGLLLDYWGQGTTVTVSS
Heavy Chain variable region (hVH2, WO 2016/033324)
(SEQ ID NO: 11)
EVQLVESGGGLIQPGGSLRLSCAASGFTFSNYGMHWVRQAPGKGLE
WVSYISSGSSTIYYADSVKGRFTISRDNSKNTLYLQMTSLRAEDTAV
YYCARRGLLLDYWGQGTTLTVSS
Heavy Chain variable region (hVH3, WO 2016/033324)
(SEQ ID NO: 45)
EVQLQESGGGVVQPGGSLRLSCAASGFTFSNYGMHWIRQAPGKGLE
WVSYISSGSSTIYYADSVKGRFTISRDNSKNTLYLQMNSLRSEDTAVY
YCARRGLLLDYWGQGTLVTVSS
Heavy Chain variable region (hVH4, WO 2016/033324)
(SEQ ID NO: 49)
EVQLVESGGGLVQPGGSLRLSCSASGFTFSNYGMHWVRQAPGKGLE
YVSYISSGSSTIYYADTVKGRFTISRDNSKNTLYLQMSSLRAEDTAVY
YCVKRGLLLDYWGQGTLVTVSS
Light Chain variable region (variants 2, 3 and 4)
(SEQ ID NO: 53)
DIQMTQSPSSLSASLGDRATITCRASKSVSTSSYSYMHWYQQKPGQP
PKLLIKYASYLESGVPSRFSGSGSGTDFTLTISSLQPEDAATYYCQHSR
EFPWTFGGGTKVEIK
Light Chain variable region (variants 6, 7 and 8)
(SEQ ID NO: 54)
DIQMTQSPSSLSASLGDRATITCRASKSVSTSSYSYMHWYQQKPGQA
PKLLIKYASYLESGVPSRFSGSGSGTDFTLTISSLQPEDFATYYCQHSR
EFPWTFGQGTKVEIK
Light Chain variable region (variants 10, 11
and 12)
(SEQ ID NO: 55)
DIQMTQSPSSLSASVGDRVTITCRASKSVSTSSYSYMHWYQQKPGKA
PKLLIKYASYLESGVPSRFSGSGSGTDFTLTISSLQPEDFATYYCQHSR
EFPWTFGQGTKVEIK
Light Chain variable region (variants 13, 14
and 15)
(SEQ ID NO: 56)
DIQMTQSPSSLSASLGDRATITCRASKTVSTSSYSYMHWYQQKPGQP
PKLLIKYASYLESGVPSRFSGSGSGTDFTLTISSLQPEDAATYYCQHSR
EFPWTFGGGTKVEIK
Light Chain variable region (variants 16, 17
and 18)
(SEQ ID NO: 57)
DIQMTQSPSSLSASVGDRVTITCRASKTVSTSSYSYMHWYQQKPGKA
PKLLIKYASYLESGVPSRFSGSGSGTDFTLTISSLQPEDFATYYCQHSR
EFPWTFGQGTKVEIK
Light Chain variable region (variant 19)
(SEQ ID NO: 58)
DIQMTQSPSSLSASLGDRATITCRASKTVSTSSYSYMHWYQQKPGQA
PKLLIKYASYLESGVPSRFSGSGSGTDFTLTISSLQPEDFATYYCQHSR
EFPWTFGQGTKVEIK
Light Chain variable region (hVL1, WO 2016/033324)
(SEQ ID NO: 3)
DIQMTQSPSSLSASVGDRVTITCRASKSVSTSSYSYLAWYQQKPEKAP
KLLIKYASYLQSGVPSRFSGSGSGTDFTLTISSLQPEDFATYYCQHSRE
FPWTFGAGTKLELK
Light Chain variable region (hVL2, WO 2016/033324)
(SEQ ID NO: 4)
DIQMTQSPSSLSASVGDRVTISCRASKSVSTSSYSYMHWYQQKPEKA
PKLLIKYASYLQSGVPSRFSGSGSGTDFTLTISSLQPEDVATYYCQHSR
EFPWTFGAGTKLELK
Light Chain variable region (hVL3, WO 2016/033324)
(SEQ ID NO: 5)
DIVLTQSPASLAVSPGQRATITCRASKSVSTSSYSYMHWYQQKPGQP
PKLLIYYASYLESGVPARFSGSGSGTDFTLTINPVEANDTANYYCQHS
REFPWTFGQGTKVEIK
Linker sequence 1
(SEQ ID NO: 59)
RADAAPGGGGSGGGGSGGGGS
Linker sequence 2
(SEQ ID NO: 60)
ASTKGPSVFPLAPLESSGS
Variant 2
(SEQ ID NO: 61)
DIQMTQSPSSLSASLGDRATITCRASKSVSTSSYSYMHWYQQKPGQP
PKLLIKYASYLESGVPSRFSGSGSGTDFTLTISSLQPEDAATYYCQHSR
EFPWTFGGGTKVEIKRADAAPGGGGSGGGGSGGGGSEVQLVESGGG
LVQPGGSLRLSCAASGFTFSNYGMHWVRQAPGKGLEWVSYISSSSST
IYYADSVKGRFTISRDNAKNSLYLQMNSLRAEDTAVYYCARRGLLL
DYWGQGTTVTVSSASTKGPSVFPLAPLESSGSDIQMTQSPSSLSASLG
DRATITCRASKSVSTSSYSYMHWYQQKPGQPPKLLIKYASYLESGVP
SRFSGSGSGTDFTLTISSLQPEDAATYYCQHSREFPWTFGGGTKVEIK
RADAAPGGGGSGGGGSGGGGSEVQLVESGGGLVQPGGSLRLSCAAS
GFTFSNYGMHWVRQAPGKGLEWVSYISSSSSTIYYADSVKGRFTISR
DNAKNSLYLQMNSLRAEDTAVYYCARRGLLLDYWGQGTTVTVSS
Variant 3
(SEQ ID NO: 62)
DIQMTQSPSSLSASLGDRATITCRASKSVSTSSYSYMHWYQQKPGQP
PKLLIKYASYLESGVPSRFSGSGSGTDFTLTISSLQPEDAATYYCQHSR
EFPWTFGGGTKVEIKRADAAPGGGGSGGGGSGGGGSEVQLVESGGG
VVQPGGSLRLSCAASGFTFSNYGMHWVRQAPEKGLEWVSYISSSSST
IYYADSVKGRFTISRDNSKNTLYLQMNSLRAEDTAVYYCARRGLLL
DYWGQGTTVTVSSASTKGPSVFPLAPLESSGSDIQMTQSPSSLSASLG
DRATITCRASKSVSTSSYSYMHWYQQKPGQPPKLLIKYASYLESGVP
SRFSGSGSGTDFTLTISSLQPEDAATYYCQHSREFPWTFGGGTKVEIK
RADAAPGGGGGGGGSGGGGSEVQLVESGGGVVQPGGSLRLSCAAS
GFTFSNYGMHWVRQAPEKGLEWVSYISSSSSTIYYADSVKGRFTISR
DNSKNTLYLQMNSLRAEDTAVYYCARRGLLLDYWGQGTTVTVSS
Variant 4
(SEQ ID NO: 63)
DIQMTQSPSSLSASLGDRATITCRASKSVSTSSYSYMHWYQQKPGQP
PKLLIKYASYLESGVPSRFSGSGSGTDFTLTISSLQPEDAATYYCQHSR
EFPWTFGGGTKVEIKRADAAPGGGGSGGGGSGGGGSEVQLVESGGG
DVKPGGSLRLSCAASGFTFSNYGMHWVRQAPEKGLEWVSYISSSSST
IYYADSVKGRFTISRDNSKNTLYLQMNSLRAEDTAVYYCARRGLLL
DYWGQGTTVTVSSASTKGPSVFPLAPLESSGSDIQMTQSPSSLSASLG
DRATITCRASKSVSTSSYSYMHWYQQKPGQPPKLLIKYASYLESGVP
SRFSGSGSGTDFTLTISSLQPEDAATYYCQHSREFPWTFGGGTKVEIK
RADAAPGGGGSGGGGSGGGGSEVQLVESGGGDVKPGGSLRLSCAAS
GFTFSNYGMHWVRQAPEKGLEWVSYISSSSSTIYYADSVKGRFTISR
DNSKNTLYLQMNSLRAEDTAVYYCARRGLLLDYWGQGTTVTVSS
Variant 6
(SEQ ID NO: 64)
DIQMTQSPSSLSASLGDRATITCRASKSVSTSSYSYMHWYQQKPGQA
PKLLIKYASYLESGVPSRFSGSGSGTDFTLTISSLQPEDFATYYCQHSR
EFPWTFGQGTKVEIKRADAAPGGGGSGGGGSGGGGSEVQLVESGGG
LVQPGGSLRLSCAASGFTFSNYGMHWVRQAPGKGLEWVSYISSSSST
IYYADSVKGRFTISRDNAKNSLYLQMNSLRAEDTAVYYCARRGLLL
DYWGQGTTVTVSSASTKGPSVFPLAPLESSGSDIQMTQSPSSLSASLG
DRATITCRASKSVSTSSYSYMHWYQQKPGQAPKLLIKYASYLESGVP
SRFSGSGSGTDFTLTISSLQPEDFATYYCQHSREFPWTFGQGTKVEIK
RADAAPGGGGSGGGGSGGGGSEVQLVESGGGLVQPGGSLRLSCAAS
GFTFSNYGMHWVRQAPGKGLEWVSYISSSSSTIYYADSVKGRFTISR
DNAKNSLYLQMNSLRAEDTAVYYCARRGLLLDYWGQGTTVTVSS
Variant 7
(SEQ ID NO: 65)
DIQMTQSPSSLSASLGDRATITCRASKSVSTSSYSYMHWYQQKPGQA
PKLLIKYASYLESGVPSRFSGSGSGTDFTLTISSLQPEDFATYYCQHSR
EFPWTFGQGTKVEIKRADAAPGGGGSGGGGSGGGGSEVQLVESGGG
VVQPGGSLRLSCAASGFTFSNYGMHWVRQAPEKGLEWVSYISSSSST
IYYADSVKGRFTISRDNSKNTLYLQMNSLRAEDTAVYYCARRGLLL
DYWGQGTTVTVSSASTKGPSVFPLAPLESSGSDIQMTQSPSSLSASLG
DRATITCRASKSVSTSSYSYMHWYQQKPGQAPKLLIKYASYLESGVP
SRFSGSGSGTDFTLTISSLQPEDFATYYCQHSREFPWTFGQGTKVEIK
RADAAPGGGGSGGGGSGGGGSEVQLVESGGGVVQPGGSLRLSCAAS
GFTFSNYGMHWVRQAPEKGLEWVSYISSSSSTIYYADSVKGRFTISR
DNSKNTLYLQMNSLRAEDTAVYYCARRGLLLDYWGQGTTVTVSS
Variant 8
(SEQ ID NO: 66)
DIQMTQSPSSLSASLGDRATITCRASKSVSTSSYSYMHWYQQKPGQA
PKLLIKYASYLESGVPSRFSGSGSGTDFTLTISSLQPEDFATYYCQHSR
EFPWTFGQGTKVEIKRADAAPGGGGSGGGGSGGGGSEVQLVESGGG
DVKPGGSLRLSCAASGFTFSNYGMHWVRQAPEKGLEWVSYISSSSST
IYYADSVKGRFTISRDNSKNTLYLQMNSLRAEDTAVYYCARRGLLL
DYWGQGTTVTVSSASTKGPSVFPLAPLESSGSDIQMTQSPSSLSASLG
DRATITCRASKSVSTSSYSYMHWYQQKPGQAPKLLIKYASYLESGVP
SRFSGSGSGTDFTLTISSLQPEDFATYYCQHSREFPWTFGQGTKVEIK
RADAAPGGGGSGGGGSGGGGSEVQLVESGGGDVKPGGSLRLSCAAS
GFTFSNYGMHWVRQAPEKGLEWVSYISSSSSTIYYADSVKGRFTISR
DNSKNTLYLQMNSLRAEDTAVYYCARRGLLLDYWGQGTTVTVSS
Variant 10
(SEQ ID NO: 67)
DIQMTQSPSSLSASVGDRVTITCRASKSVSTSSYSYMHWYQQKPGKA
PKLLIKYASYLESGVPSRFSGSGSGTDFTLTISSLQPEDFATYYCQHSR
EFPWTFGQGTKVEIKRADAAPGGGGSGGGGSGGGGSEVQLVESGGG
LVQPGGSLRLSCAASGFTFSNYGMHWVRQAPGKGLEWVSYISSSSST
IYYADSVKGRFTISRDNAKNSLYLQMNSLRAEDTAVYYCARRGLLL
DYWGQGTTVTVSSASTKGPSVFPLAPLESSGSDIQMTQSPSSLSASVG
DRVTITCRASKSVSTSSYSYMHWYQQKPGKAPKLLIKYASYLESGVP
SRFSGSGSGTDFTLTISSLQPEDFATYYCQHSREFPWTFGQGTKVEIK
RADAAPGGGGSGGGGSGGGGSEVQLVESGGGLVQPGGSLRLSCAAS
GFTFSNYGMHWVRQAPGKGLEWVSYISSSSSTIYYADSVKGRFTISR
DNAKNSLYLQMNSLRAEDTAVYYCARRGLLLDYWGQGTTVTVSS
Variant 11
(SEQ ID NO: 68)
DIQMTQSPSSLSASVGDRVTITCRASKSVSTSSYSYMHWYQQKPGKA
PKLLIKYASYLESGVPSRFSGSGSGTDFTLTISSLQPEDFATYYCQHSR
EFPWTFGQGTKVEIKRADAAPGGGGSGGGGSGGGGSEVQLVESGGG
VVQPGGSLRLSCAASGFTFSNYGMHWVRQAPEKGLEWVSYISSSSST
IYYADSVKGRFTISRDNSKNTLYLQMNSLRAEDTAVYYCARRGLLL
DYWGQGTTVTVSSASTKGPSVFPLAPLESSGSDIQMTQSPSSLSASVG
DRVTITCRASKSVSTSSYSYMHWYQQKPGKAPKLLIKYASYLESGVP
SRFSGSGSGTDFTLTISSLQPEDFATYYCQHSREFPWTFGQGTKVEIK
RADAAPGGGGSGGGGSGGGGSEVQLVESGGGVVQPGGSLRLSCAAS
GFTFSNYGMHWVRQAPEKGLEWVSYISSSSSTIYYADSVKGRFTISR
DNSKNTLYLQMNSLRAEDTAVYYCARRGLLLDYWGQGTTVTVSS
Variant 12
(SEQ ID NO: 69)
DIQMTQSPSSLSASVGDRVTITCRASKSVSTSSYSYMHWYQQKPGKA
PKLLIKYASYLESGVPSRFSGSGSGTDFTLTISSLQPEDFATYYCQHSR
EFPWTFGQGTKVEIKRADAAPGGGGSGGGGSGGGGSEVQLVESGGG
DVKPGGSLRLSCAASGFTFSNYGMHWVRQAPEKGLEWVSYISSSSST
IYYADSVKGRFTISRDNSKNTLYLQMNSLRAEDTAVYYCARRGLLL
DYWGQGTTVTVSSASTKGPSVFPLAPLESSGSDIQMTQSPSSLSASVG
DRVTITCRASKSVSTSSYSYMHWYQQKPGKAPKLLIKYASYLESGVP
SRFSGSGSGTDFTLTISSLQPEDFATYYCQHSREFPWTFGQGTKVEIK
RADAAPGGGGSGGGGSGGGGSEVQLVESGGGDVKPGGSLRLSCAAS
GFTFSNYGMHWVRQAPEKGLEWVSYISSSSSTIYYADSVKGRFTISR
DNSKNTLYLQMNSLRAEDTAVYYCARRGLLLDYWGQGTTVTVSS
Variant 13
(SEQ ID NO: 70)
DIQMTQSPSSLSASLGDRATITCRASKTVSTSSYSYMHWYQQKPGQPP
KLLIKYASYLESGVPSRFSGSGSGTDFTLTISSLQPEDAATYYCQHSRE
FPWTFGGGTKVEIKRADAAPGGGGSGGGGSGGGGSEVQLVESGGGL
VQPGGSLRLSCAASGFTFSNYGMHWVRQAPGKGLEWVSYISSGSSTI
YYADSVKGRFTISRDNAKNSLYLQMNSLRAEDTAVYYCARRGLLLD
YWGQGTTVTVSSASTKGPSVFPLAPLESSGSDIQMTQSPSSLSASLGDR
ATITCRASKTVSTSSYSYMHWYQQKPGQPPKLLIKYASYLESGVPSRF
SGSGSGTDFTLTISSLQPEDAATYYCQHSREFPWTFGGGTKVEIKRAD
AAPGGGGSGGGGSGGGGSEVQLVESGGGLVQPGGSLRLSCAASGFTF
SNYGMHWVRQAPGKGLEWVSYISSGSSTIYYADSVKGRFTISRDNAK
NSLYLQMNSLRAEDTAVYYCARRGLLLDYWGQGTTVTVSS
Variant 14
(SEQ ID NO: 71)
DIQMTQSPSSLSASLGDRATITCRASKTVSTSSYSYMHWYQQKPGQP
PKLLIKYASYLESGVPSRFSGSGSGTDFTLTISSLQPEDAATYYCQHSR
EFPWTFGGGTKVEIKRADAAPGGGGSGGGGSGGGGSEVQLVESGGG
VVQPGGSLRLSCAASGFTFSNYGMHWVRQAPEKGLEWVSYISSGSS
TIYYADSVKGRFTISRDNSKNTLYLQMNSLRAEDTAVYYCARRGLLL
DYWGQGTTVTVSSASTKGPSVFPLAPLESSGSDIQMTQSPSSLSASLG
DRATITCRASKTVSTSSYSYMHWYQQKPGQPPKLLIKYASYLESGVP
SRFSGSGSGTDFTLTISSLQPEDAATYYCQHSREFPWTFGGGTKVEIK
RADAAPGGGGSGGGGSGGGGSEVQLVESGGGVVQPGGSLRLSCAAS
GFTFSNYGMHWVRQAPEKGLEWVSYISSGSSTIYYADSVKGRFTISR
DNSKNTLYLQMNSLRAEDTAVYYCARRGLLLDYWGQGTTVTVSS
Variant 15
(SEQ ID NO: 72)
DIQMTQSPSSLSASLGDRATITCRASKTVSTSSYSYMHWYQQKPGQP
PKLLIKYASYLESGVPSRFSGSGSGTDFTLTISSLQPEDAATYYCQHSR
EFPWTFGGGTKVEIKRADAAPGGGGSGGGGSGGGGSEVQLVESGGG
DVKPGGSLRLSCAASGFTFSNYGMHWVRQAPEKGLEWVSYISSGSS
TIYYADSVKGRFTISRDNSKNTLYLQMNSLRAEDTAVYYCARRGLLL
DYWGQGTTVTVSSASTKGPSVFPLAPLESSGSDIQMTQSPSSLSASLG
DRATITCRASKTVSTSSYSYMHWYQQKPGQPPKLLIKYASYLESGVP
SRFSGSGSGTDFTLTISSLQPEDAATYYCQHSREFPWTFGGGTKVEIK
RADAAPGGGGSGGGGSGGGGSEVQLVESGGGDVKPGGSLRLSCAAS
GFTFSNYGMHWVRQAPEKGLEWVSYISSGSSTIYYADSVKGRFTISR
DNSKNTLYLQMNSLRAEDTAVYYCARRGLLLDYWGQGTTVTVSS
Variant 16
(SEQ ID NO: 73)
DIQMTQSPSSLSASVGDRVTITCRASKTVSTSSYSYMHWYQQKPGKA
PKLLIKYASYLESGVPSRFSGSGSGTDFTLTISSLQPEDFATYYCQHSR
EFPWTFGQGTKVEIKRADAAPGGGGSGGGGSGGGGSEVQLVESGGG
LVQPGGSLRLSCAASGFTFSNYGMHWVRQAPGKGLEWVSYISSGSS
TIYYADSVKGRFTISRDNAKNSLYLQMNSLRAEDTAVYYCARRGLL
LDYWGQGTTVTVSSASTKGPSVFPLAPLESSGSDIQMTQSPSSLSASV
GDRVTITCRASKTVSTSSYSYMHWYQQKPGKAPKLLIKYASYLESGV
PSRFSGSGSGTDFTLTISSLQPEDFATYYCQHSREFPWTFGQGTKVEIK
RADAAPGGGGSGGGGSGGGGSEVQLVESGGGLVQPGGSLRLSCAAS
GFTFSNYGMHWVRQAPGKGLEWVSYISSGSSTIYYADSVKGRFTISR
DNAKNSLYLQMNSLRAEDTAVYYCARRGLLLDYWGQGTTVTVSS
Variant 17
(SEQ ID NO: 74)
DIQMTQSPSSLSASVGDRVTITCRASKTVSTSSYSYMHWYQQKPGKA
PKLLIKYASYLESGVPSRFSGSGSGTDFTLTISSLQPEDFATYYCQHSR
EFPWTFGQGTKVEIKRADAAPGGGGSGGGGSGGGGSEVQLVESGGG
VVQPGGSLRLSCAASGFTFSNYGMHWVRQAPEKGLEWVSYISSGSS
TIYYADSVKGRFTISRDNSKNTLYLQMNSLRAEDTAVYYCARRGLLL
DYWGQGTTVTVSSASTKGPSVFPLAPLESSGSDIQMTQSPSSLSASVG
DRVTITCRASKTVSTSSYSYMHWYQQKPGKAPKLLIKYASYLESGVP
SRFSGSGSGTDFTLTISSLQPEDFATYYCQHSREFPWTFGQGTKVEIK
RADAAPGGGGSGGGGSGGGGSEVQLVESGGGVVQPGGSLRLSCAAS
GFTFSNYGMHWVRQAPEKGLEWVSYISSGSSTIYYADSVKGRFTISR
DNSKNTLYLQMNSLRAEDTAVYYCARRGLLLDYWGQGTTVTVSS
Variant 18
(SEQ ID NO: 75)
DIQMTQSPSSLSASVGDRVTITCRASKTVSTSSYSYMHWYQQKPGKA
PKLLIKYASYLESGVPSRFSGSGSGTDFTLTISSLQPEDFATYYCQHSR
EFPWTFGQGTKVEIKRADAAPGGGGSGGGGSGGGGSEVQLVESGGG
DVKPGGSLRLSCAASGFTFSNYGMHWVRQAPEKGLEWVSYISSGSS
TIYYADSVKGRFTISRDNSKNTLYLQMNSLRAEDTAVYYCARRGLLL
DYWGQGTTVTVSSASTKGPSVFPLAPLESSGSDIQMTQSPSSLSASVG
DRVTITCRASKTVSTSSYSYMHWYQQKPGKAPKLLIKYASYLESGVP
SRFSGSGSGTDFTLTISSLQPEDFATYYCQHSREFPWTFGQGTKVEIK
RADAAPGGGGSGGGGSGGGGSEVQLVESGGGDVKPGGSLRLSCAAS
GFTFSNYGMHWVRQAPEKGLEWVSYISSGSSTIYYADSVKGRFTISR
DNSKNTLYLQMNSLRAEDTAVYYCARRGLLLDYWGQGTTVTVSS
Variant 19
(SEQ ID NO: 76)
DIQMTQSPSSLSASLGDRATITCRASKTVSTSSYSYMHWYQQKPGQA
PKLLIKYASYLESGVPSRFSGSGSGTDFTLTISSLQPEDFATYYCQHSR
EFPWTFGQGTKVEIKRADAAPGGGGSGGGGSGGGGSEVQLVESGGG
LVQPGGSLRLSCAASGFTFSNYGMHWVRQAPGKGLEWVSYISSGSS
TIYYADSVKGRFTISRDNAKNSLYLQMNSLRAEDTAVYYCARRGLL
LDYWGQGTTVTVSSASTKGPSVFPLAPLESSGSDIQMTQSPSSLSASL
GDRATITCRASKTVSTSSYSYMHWYQQKPGQAPKLLIKYASYLESGV
PSRFSGSGSGTDFTLTISSLQPEDFATYYCQHSREFPWTFGQGTKVEIK
RADAAPGGGGSGGGGSGGGGSEVQLVESGGGLVQPGGSLRLSCAAS
GFTFSNYGMHWVRQAPGKGLEWVSYISSGSSTIYYADSVKGRFTISR
DNAKNSLYLQMNSLRAEDTAVYYCARRGLLLDYWGQGTTVTVSS
In another embodiment, a humanized Fv3E10
includes
(SEQ ID NO: 88)
DIVLTQSPASLAVSPGQRATITCRASKSVSTSSYSYMHWYQQKPGQP
PKLLIYYASYLESGVPARFSGSGSGTDFTLTINPVEANDTANYYCQHS
REFPWTFGQGTKVEIKGGGGSGGGGSGGGGSEVQLVESGGGLVQPG
GSLRLSCSASGFTFSNYGMHWVRQAPGKGLEYVSYISSGSSTIYYAD
TVKGRFTISRDNSKNTLYLQMSSLRAEDTAVYYCVKRGLLLDYWGQ
GTLVTVSS (Fv3E10, WO 2016/033324))
IgG1 L2345A/L235A heavy chain full length
sequence
(SEQ ID NO: 77)
EVQLVESGGGLVQPGGSLRLSCAASGFTFSNYGMHWVRQAPGKGLE
WVSYISSGSSTIYYADSVKGRFTISRDNAKNSLYLQMNSLRAEDTAV
YYCARRGLLLDYWGQGTTVTVSSASTKGPSVFPLAPSSKSTSGGTAA
LGCLVKDYFPEPVTVSWNSGALTSGVHTFPAVLQSSGLYSLSSVVTV
PSSSLGTQTYICNVNHKPSNTKVDKKVEPKSCDKTHTCPPCPAPEAA
GGPSVFLFPPKPKDTLMISRTPEVTCVVVDVSHEDPEVKFNWYVDGV
EVHNAKTKPREEQYNSTYRVVSVLTVLHQDWLNGKEYKCKVSNKA
LPAPIEKTISKAKGQPREPQVYTLPPSRDELTKNQVSLTCLVKGFYPS
DIAVEWESNGQPENNYKTTPPVLDSDGSFFLYSKLTVDKSRWQQGN
VFSCSVMHEALHNHYTQKSLSLSPGK
IgG1 constant heavy region 1
(SEQ ID NO: 78)
ASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVD
KKV
IgG1 hinge region
(SEQ ID NO: 79)
EPKSCDKTHTCP
IgG1 L2345A/L235A constant heavy region 2
(SEQ ID NO: 80)
PCPAPEAAGGPSVFLFPPKPKDTLMISRTPEVTCVVVDVSHEDPEVKF
NWYVDGVEVHNAKTKPREEQYNSTYRVVSVLTVLHQDWLNGKEY
KCKVSNKALPAPIEKTISKAK
IgG1 constant heavy region 3
(SEQ ID NO: 81)
GQPREPQVYTLPPSRDELTKNQVSLTCLVKGFYPSDIAVEWESNGQP
ENNYKTTPPVLDSDGSFFLYSKLTVDKSRWQQGNVFSCSVMHEALH
NHYTQKSLSLSPGK
IgG1 N297D heavy chain full length sequence
(SEQ ID NO: 82)
EVQLVESGGGLVQPGGSLRLSCAASGFTFSNYGMHWVRQAPGKGLE
WVSYISSGSSTIYYADSVKGRFTISRDNAKNSLYLQMNSLRAEDTAV
YYCARRGLLLDYWGQGTTVTVSSASTKGPSVFPLAPSSKSTSGGTAA
LGCLVKDYFPEPVTVSWNSGALTSGVHTFPAVLQSSGLYSLSSVVTV
PSSSLGTQTYICNVNHKPSNTKVDKKVEPKSCDKTHTCPPCPAPELLG
GPSVFLFPPKPKDTLMISRTPEVTCVVVDVSHEDPEVKFNWYVDGVE
VHNAKTKPREEQYDSTYRVVSVLTVLHQDWLNGKEYKCKVSNKAL
PAPIEKTISKAKGQPREPQVYTLPPSRDELTKNQVSLTCLVKGFYPSDI
AVEWESNGQPENNYKTTPPVLDSDGSFFLYSKLTVDKSRWQQGNVF
SCSVMHEALHNHYTQKSLSLSPGK
IgG1 N297D constant heavy region 2
(SEQ ID NO: 83)
PCPAPELLGGPSVFLFPPKPKDTLMISRTPEVTCVVVDVSHEDPEVKF
NWYVDGVEVHNAKTKPREEQYDSTYRVVSVLTVLHQDWLNGKEY
KCKVSNKALPAPIEKTISKAK
IgG1 L2345A/L235A/N297D heavy chain full length
sequence
(SEQ ID NO: 84)
EVQLVESGGGLVQPGGSLRLSCAASGFTFSNYGMHWVRQAPGKGLE
WVSYISSGSSTIYYADSVKGRFTISRDNAKNSLYLQMNSLRAEDTAV
YYCARRGLLLDYWGQGTTVTVSSASTKGPSVFPLAPSSKSTSGGTAA
LGCLVKDYFPEPVTVSWNSGALTSGVHTFPAVLQSSGLYSLSSVVTV
PSSSLGTQTYICNVNHKPSNTKVDKKVEPKSCDKTHTCPPCPAPEAA
GGPSVFLFPPKPKDTLMISRTPEVTCVVVDVSHEDPEVKFNWYVDGV
EVHNAKTKPREEQYDSTYRVVSVLTVLHQDWLNGKEYKCKVSNKA
LPAPIEKTISKAKGQPREPQVYTLPPSRDELTKNQVSLTCLVKGFYPS
DIAVEWESNGQPENNYKTTPPVLDSDGSFFLYSKLTVDKSRWQQGN
VFSCSVMHEALHNHYTQKSLSLSPGK
(SEQ ID NO: 85)
IgG1 L2345A/L235A/N297D constant heavy region 2
PCPAPEAAGGPSVFLFPPKPKDTLMISRTPEVTCVVVDVSHEDPEVKF
NWYVDGVEVHNAKTKPREEQYDSTYRVVSVLTVLHQDWLNGKEY
KCKVSNKALPAPIEKTISKAK
(SEQ ID NO: 86)
Unmodified constant heavy region 2
PCPAPELLGGPSVFLFPPKPKDTLMISRTPEVTCVVVDVSHEDPEVKF
NWYVDGVEVHNAKTKPREEQYNSTYRVVSVLTVLHQDWLNGKEY
KCKVSNKALPAPIEKTISKAK
Light chain full length sequence
(SEQ ID NO: 87)
DIQMTQSPSSLSASLGDRATITCRASKTVSTSSYSYMHWYQQKPGQP
PKLLIKYASYLESGVPSRFSGSGSGTDFTLTISSLQPEDAATYYCQHSR
EFPWTFGGGTKLEIKRTVAAPSVFIFPPSDEQLKSGTASVVCLLNNFY
PREAKVQWKVDNALQSGNSQESVTEQDSKDSTYSLSSTLTLSKADY
EKHKVYACEVTHQGLSSPVTKSFNRGEC

f. Additional Exemplary Anti-DNA scFv Sequences

Exemplary murine 3E10 scFv sequences, including mono-, di-, and tri-scFv are disclosed in WO 2016/033321 and WO 2017/218825 and provided below. Cell-penetrating antibodies for use in the disclosed combination therapies include exemplary scFv, and fragments and variants thereof.

The amino acid sequence for scFv 3E10 (D31N) is:

(SEQ ID NO: 27)
AGIHDIVLTQSPASLAVSLGQRATISCRASKSVSTSSYSYMHWYQQK
PGQPPKLLIKYASYLESGVPARFSGSGSGTDFTLNIHPVEEEDAATYY
CQHSREFPWTFGGGTKLEIKRADAAPGGGGSGGGGSGGGGSEVQLV
ESGGGLVKPGGSRKLSCAASGFTFSNYGMHWVRQAPEKGLEWVAYI
SSGSSTIYYADTVKGRFTISRDNAKNTLFLQMTSLRSEDTAMYYCAR
RGLLLDYWGQGTTLTVSSLEQKLISEEDLNSAVDHHHHHH.

### Annotation of scFv Protein Domains with Reference to SEQ ID NO:27

AGIH sequence increases solubility (amino acids 1-4 of SEQ ID NO:27)
Vk variable region (amino acids 5-115 of SEQ ID NO:27)
Initial (6 aa) of light chain CH1 (amino acids 116-121 of SEQ ID NO:27)
(GGGGS)₃ (SEQ ID NO:26) linker (amino acids 122-136 of SEQ ID NO:27)
VH variable region (amino acids 137-252 of SEQ ID NO:27)
Myc tag (amino acids 253-268 SEQ ID NO:27)
His 6 tag (amino acids 269-274 of SEQ ID NO:27)

### Amino Acid Sequence of 3E10 Di-scFv (D31N)

Di-scFv 3E10 (D31N) is a di-single chain variable fragment including 2× the heavy chain and light chain variable regions of 3E10 and wherein the aspartic acid at position 31 of the heavy chain is mutated to an asparagine. The amino acid sequence for di-scFv 3E10 (D31N) is:

(SEQ ID NO: 28)
AGIHDIVLTQSPASLAVSLGQRATISCRASKSVSTSSYSYMHWYQQK
PGQPPKLLIKYASYLESGVPARFSGSGSGTDFTLNIHPVEEEDAATYY
CQHSREFPWTFGGGTKLEIKRADAAPGGGGSGGGGSGGGGSEVQLV
ESGGGLVKPGGSRKLSCAASGFTFSNYGMHWVRQAPEKGLEWVAYI
SSGSSTIYYADTVKGRFTISRDNAKNTLFLQMTSLRSEDTAMYYCAR
RGLLLDYWGQGTTLTVSSASTKGPSVFPLAPLESSGSDIVLTQSPASL
AVSLGQRATISCRASKSVSTSSYSYMHWYQQKPGQPPKLLIKYASYL
ESGVPARFSGSGSGTDFTLNIHPVEEEDAATYYCQHSREFPWTFGGG
TKLEIKRADAAPGGGGSGGGGSGGGGSEVQLVESGGGLVKPGGSRK
LSCAASGFTFSNYGMHWVRQAPEKGLEWVAYISSGSSTIYYADTVK
GRFTISRDNAKNTLFLQMTSLRSEDTAMYYCARRGLLLDYWGQGTT
LTVSSLEQKLISEEDLNSAVDHHHHHH.

### Annotation of Di-scFv Protein Domains with Reference to SEQ ID NO:28

AGIH sequence increases solubility (amino acids 1-4 of SEQ ID NO:28)
Vk variable region (amino acids 5-115 of SEQ ID NO:28)
Initial (6 aa) of light chain CH1 (amino acids 116-121 of SEQ ID NO:28)
(GGGGS)₃ (SEQ ID NO:26) linker (amino acids 122-136 of SEQ ID NO:28)
VH variable region (amino acids 137-252 of SEQ ID NO:28)
Linker between Fv fragments consisting of human IgG CH1 initial 13 amino acids (amino acids 253-265 of SEQ ID NO:28)
Swivel sequence (amino acids 266-271 of SEQ ID NO:28)
Vk variable region (amino acids 272-382 of SEQ ID NO:28)
Initial (6 aa) of light chain CH1 (amino acids 383-388 of SEQ ID NO:28)
(GGGGS)₃ (SEQ ID NO:26) linker (amino acids 389-403 of SEQ ID NO:28)
VH variable region (amino acids 404-519 of SEQ ID NO:28)
Myc tag (amino acids 520-535 of SEQ ID NO:28)
His 6 tag (amino acids 536-541 of SEQ ID NO:28)

### Amino Acid Sequence for Tri-scFv

Tri-scFv 3E10 (D31N) is a tri-single chain variable fragment including 3× the heavy chain and light chain variable regions of 310E and wherein the aspartic acid at position 31 of the heavy chain is mutated to an asparagine. The amino acid sequence for tri-scFv 3E10 (D31N) is:

(SEQ ID NO: 29)
AGIHDIVLTQSPASLAVSLGQRATISCRASKSVSTSSYSYMHWYQQK
PGQPPKLLIKYASYLESGVPARFSGSGSGTDFTLNIHPVEEEDAATYY
CQHSREFPWTFGGGTKLEIKRADAAPGGGGSGGGGSGGGGSEVQLV
ESGGGLVKPGGSRKLSCAASGFTFSNYGMHWVRQAPEKGLEWVAYI
SSGSSTIYYADTVKGRFTISRDNAKNTLFLQMTSLRSEDTAMYYCAR
RGLLLDYWGQGTTLTVSSASTKGPSVFPLAPLESSGSDIVLTQSPASL
AVSLGQRATISCRASKSVSTSSYSYMHWYQQKPGQPPKLLIKYASYL
ESGVPARFSGSGSGTDFTLNIHPVEEEDAATYYCQHSREFPWTFGGG
TKLEIKRADAAPGGGGSGGGGSGGGGSEVQLVESGGGLVKPGGSRK
LSCAASGFTFSNYGMHWVRQAPEKGLEWVAYISSGSSTIYYADTVK
GRFTISRDNAKNTLFLQMTSLRSEDTAMYYCARRGLLLDYWGQGTT
LTVSSASTKGPSVFPLAPLESSGSDIVLTQSPASLAVSLGQRATISCRA
SKSVSTSSYSYMHWYQQKPGQPPKLLIKYASYLESGVPARFSGSGSG
TDFTLNIHPVEEEDAATYYCQHSREFPWTFGGGTKLEIKRADAAPGG
GGSGGGGSGGGGSEVQLVESGGGLVKPGGSRKLSCAASGFTFSNYG
MHWVRQAPEKGLEWVAYISSGSSTIYYADTVKGRFTISRDNAKNTLF
LQMTSLRSEDTAMYYCARRGLLLDYWGQGTTLTVSSLEQKLISEED
LNSAVDHHHHHH.

### Annotation of Tri-scFv Protein Domains with Reference to SEQ ID NO:29

AGIH sequence increases solubility (amino acids 1˜4 of SEQ ID NO:29)
Vk variable region (amino acids 5-115 of SEQ ID NO:29)
Initial (6 aa) of light chain CH1 (amino acids 116-121 of SEQ ID NO:29)
(GGGGS)₃ (SEQ ID NO:26) linker (amino acids 122-136 of SEQ ID NO:29)
VH variable region (amino acids 137-252 of SEQ ID NO:29)
Linker between Fv fragments consisting of human IgG CH1 initial 13 amino acids (amino acids 253-265 of SEQ ID NO:29)
Swivel sequence (amino acids 266-271 of SEQ ID NO:29)
Vk variable region (amino acids 272-382 of SEQ ID NO:29)
Initial (6 aa) of light chain CH1 (amino acids 383-388 of SEQ ID NO:29)
(GGGGS)₃ (SEQ ID NO:26) linker (amino acids 389-403 of SEQ ID NO:29
VH variable region (amino acids 404-519 of SEQ ID NO:29)
Linker between Fv fragments consisting of human IgG C_{H}1 initial 13 amino acids (amino acids 520-532 of SEQ ID NO:29)
Swivel sequence (amino acids 533-538 of SEQ ID NO:29)
Vk variable region (amino acids 539-649 of SEQ ID NO:29)
Initial (6 aa) of light chain CH1 (amino acids 650-655 of SEQ ID NO:29)
(GGGGS)₃ (SEQ ID NO:26) linker (amino acids 656-670 of SEQ ID NO:29)
VH variable region (amino acids 671-786 of SEQ ID NO:29)
Myc tag (amino acids 787-802 of SEQ ID NO:29)
His 6 tag (amino acids 803-808 of SEQ ID NO:29)

WO 2016/033321 and Noble, et al., Cancer Research, 75(11):2285-2291 (2015), show that di-scFv and tri-scFv have some improved and additional activities compared to their monovalent counterpart. The subsequences corresponding to the different domains of each of the exemplary fusion proteins are also provided above. One of skill in the art will appreciate that the exemplary fusion proteins, or domains thereof, can be utilized to construct fusion proteins discussed in more detail above. For example, in some embodiments, the di-scFv includes a first scFv including a Vk variable region (e.g., amino acids 5-115 of SEQ ID NO:28, or a functional variant or fragment thereof), linked to a VH variable domain (e.g., amino acids 137-252 of SEQ ID NO:28, or a functional variant or fragment thereof), linked to a second scFv including a Vk variable region (e.g., amino acids 272-382 of SEQ ID NO:28, or a functional variant or fragment thereof), linked to a VH variable domain (e.g., amino acids 404-519 of SEQ ID NO:28, or a functional variant or fragment thereof). In some embodiments, a tri-scFv includes a di-scFv linked to a third scFv domain including a Vk variable region (e.g., amino acids 539-649 of SEQ ID NO:29, or a functional variant or fragment thereof), linked to a VH variable domain (e.g., amino acids 671-786 of SEQ ID NO:29, or a functional variant or fragment thereof).

The Vk variable regions can be linked to VH variable domains by, for example, a linker (e.g., (GGGGS) 3 (SEQ ID NO:26), alone or in combination with a (6 aa) of light chain CH1 (amino acids 116-121 of SEQ ID NO:28). Other suitable linkers are discussed above and known in the art. scFv can be linked by a linker (e.g., human IgG CH1 initial 13 amino acids (253-265) of SEQ ID NO:28), alone or in combination with a swivel sequence (e.g., amino acids 266-271 of SEQ ID NO:28). Other suitable linkers are discussed above and known in the art.

Therefore, a di-scFv can include amino acids 5-519 of SEQ ID NO:28. A tri-scFv can include amino acids 5-786 of SEQ ID NO:29. In some embodiments, the fusion proteins include additional domains. For example, in some embodiments, the fusion proteins include sequences that enhance solubility (e.g., amino acids 1-4 of SEQ ID NO:28). Therefore, in some embodiments, a di-scFv can include amino acids 1-519 of SEQ ID NO:28. A tri-scFv can include amino acids 1-786 of SEQ ID NO:29. In some embodiments that fusion proteins include one or more domains that enhance purification, isolation, capture, identification, separation, etc., of the fusion protein. Exemplary domains include, for example, Myc tag (e.g., amino acids 520-535 of SEQ ID NO:28) and/or a His tag (e.g., amino acids 536-541 of SEQ ID NO:28). Therefore, in some embodiments, a di-scFv can include the amino acid sequence of SEQ ID NO:28. A tri-scFv can include the amino acid sequence of SEQ ID NO:29. Other substitutable domains and additional domains are discussed in more detail above.

The disclosed compositions and methods can be further understood through the following numbered paragraphs.

1. A method of treating an autoimmune disease comprising administering to a subject in need thereof an effective amount of an inhibitor of a nucleoside transporter to reduce transcellular transport of one or more cell penetrating antibodies into a tissue of the subject.

2. The method of paragraph 1, wherein the tissue is the brain.

3. The method of paragraphs 1 and 2, wherein the inhibitor reduces transport of the antibody or antibodies across the blood-brain barrier (BBB).

4. The method of any one of paragraphs 1-3, wherein the subject has central nervous system lupus.

5. The method of paragraph 1, wherein the tissue is the kidney.

6. The method of any one of paragraphs 1-5, wherein the autoimmune disease has one or more symptoms or pathology dependent on or otherwise caused by one or more cell penetrating antibodies.

7. The method of paragraph 6, wherein one or more of cell penetrating antibodies are nuclear penetrating antibodies.

8. The method of paragraphs 6 or 7, wherein one or more of the antibodies bind to nucleic acids, nucleotides, nucleotides, or a combination thereof.

9. The method of any one of paragraphs 1-8 wherein one or more of the antibodies are internalized and/or transcellularly transported at least in part by the nucleotide transporter.

10. The method of any one of paragraphs 1-9, wherein the autoimmune disease is one or more of systemic lupus erythematosus (lupus or SLE), CNS, lupus, systemic sclerosis (scleroderma), Graves' disease, myasthenia gravis, autoimmune hemolytic anemia, and pemphigus vulgaris, and additionally may contribute to the severity of disease in other autoimmune diseases such as rheumatoid arthritis, autoimmune thrombocytopenia, autoimmune atrophic gastritis of pernicious anemia, myasthenia gravis, Goodpasture's syndrome, diabetes mellitus, multiple sclerosis, Hashimoto's thyroiditis, Crohn's disease, and Sjögren's syndrome, primary biliary cirrhosis, primary sclerosing cholangitis, psoriasis, psoriatic arthritis, POEMS syndrome, dermatomyositis, inclusion body myositis, inflammatory myopathies, vasculitis syndromes including but not limited to Churg-Strauss Syndrome, Wegener granulomatosis, Behcet's disease, Buerger's disease, Kawasaki disease, Takayasu's arteritis, Henoch-Schonlein purpura, Giant cell arteritis, or polyarteritis nodosa.

11. The method of any one of paragraphs 1 to 10, wherein the autoimmune disease is a lupus.

12. The method of paragraph 11, wherein the lupus includes or is selected from group consisting of CNS, lupus, systemic lupus erythematosus, discoid lupus erythematosus, subacute cutaneous lupus erythematosus, neonatal lupus, and drug-induced lupus.

13. The method of paragraph 12, wherein the lupus includes CNS lupus.

14. The method of any one of paragraphs 1 to 13, wherein the autoimmune disease is scleroderma.

15. A method of treating lupus comprising administering to a subject in need thereof effective amount of an inhibitor of a nucleoside transporter to reduce one or more symptoms of the lupus.

16. The method of paragraph 15, wherein the lupus is selected from group consisting of systemic lupus erythematosus, discoid lupus erythematosus, subacute cutaneous lupus erythematosus, neonatal lupus, and drug-induced lupus.

17. A method of treating CNS lupus comprising administering to a subject in need thereof effective amount of an inhibitor of a nucleoside transporter to reduce one or more symptoms of the CNS lupus.

18. A method of treating an autoimmune disease comprising administering to a subject in need thereof an effective amount of an inhibitor of a nucleoside transporter to reduce transduction of one or more cell penetrating antibodies into cells of the subject,
wherein the inhibitor of the nucleoside transporter is dipyridamole or a tautomer, geometrical isomer, optically active form, enantiomeric mixture thereof, pharmaceutically acceptable salt or pharmaceutically active analogue thereof.

19. The method of paragraph 18, wherein the cells are BBB cells.

20. The method of paragraph 19, wherein the BBB cells are human endothelial cells.

21. The method of any one of paragraphs 18-20, wherein the tissue is brain tissue.

22. A method of reducing transcellular transport an antibody into or through a tissue in a subject comprising administering the subject an effective amount of an inhibitor of a nucleoside transporter to reduce transcellular transport of the antibody into or through the tissue,
wherein the subject is administered the antibody separately or together with the inhibitor of the nucleoside transporter,
optionally wherein the subject has cancer.

23. A method of reducing translocation of an antibody into cells of a subject comprising administering to the subject an effective amount of an inhibitor of a nucleoside transporter to reduce translocation of the antibody into the cells,
wherein the subject is administered the antibody separately or together with the inhibitor of the nucleoside transporter,
optionally wherein the subject has cancer.

24. The method of paragraphs 22 or 23, wherein the subject is administered the antibody in an effective amount to inhibit DNA repair or directly damage DNA separately or together with the inhibitor of the nucleoside transporter, optionally wherein the antibody is therapeutic for the cancer.

25. The method of any one of paragraphs 22-24, wherein the antibody is selected from the group consisting of 3E10, 5C6, fragments and fusions of 3E10 and 5C6, and variants and humanized forms of 3E10, 5C6, and fragments and fusions of 3E10 and 5C6.

26. The method of paragraph 25, wherein the antibody is a humanized 3E10 di-scFv.

27. The method of paragraph 26, wherein the humanized 3E10 di-scFv is SEQ ID NO:70, or variant thereof with 90% sequence identity thereto.

28. The method of any one of paragraphs 1-27, wherein the inhibitor of the nucleoside transporter inhibits activity or expression of one or more of ENT1, ENT2, ENT3, or ENT4, optionally at least or only ENT2.

29. The method of any one of paragraphs 1-28, wherein the inhibitor of the nucleoside transporter is a purine nucleoside analogue, a pyrimidopyrimidine or pteridine derivative, or a flazine calcium channel blocker.

30. The method of any one of paragraph 1-29, wherein the inhibitor of the nucleoside transporter is dipyridamole or a tautomer, geometrical isomer, optically active form, enantiomeric mixture thereof, pharmaceutically acceptable salt or pharmaceutically active analogue thereof.

31. The method of paragraph 30, wherein the inhibitor of the nucleoside transporter is dipyridamole or pharmaceutically acceptable salt thereof.

32. The method of paragraph 30, wherein the inhibitor of the nucleoside transporter is a dipyridamole analogue or pharmaceutically acceptable salt thereof.

33. The method of paragraph 32, wherein the dipyridamole analogue is a compound of formula I.

34. The method of paragraph 32, wherein the dipyridamole analogue is a compound of any one of Tables 1-5.

35. The method of any one of paragraphs 1-28, wherein the inhibitor of the nucleoside transporter is a peptide.

36. The method of any of paragraphs 1-28, wherein the inhibitor of the nucleoside transporter is a binding protein.

37. The method of paragraph 36, wherein the inhibitor of the nucleoside transporter is an antibody or fragment thereof.

38. The method of any of paragraphs 1-28, wherein the inhibitor of the nucleoside transporter is an oligonucleotide inhibitor.

39. The method of paragraph 38, wherein the oligonucleotide inhibitor is an antisense RNA or DNA, siRNA or siDNA, miRNA, miRNA mimic, shRNA or DNA and Chimeric Antisense DNA or RNA.

40. The method paragraph 39, wherein the inhibitor of the nucleoside transporter is an siRNA, shRNA, or miRNA.

41. The method of any one of paragraphs 1-40 wherein the inhibitor is administered to a subject in need thereof by a parenteral, enteral, transdermal, or transmucosal route of administration.

42. A composition comprising an effective amount an inhibitor of any one of paragraphs 1-41 to reduce one or more symptoms of an autoimmune disease in a subject in need thereof.

43. The composition of paragraph 42, wherein the autoimmune disease is or includes CNS lupus.

### EXAMPLES

### Example 1: h3E10 Di-scFv Suppresses Tumor Growth and Prolongs Survival in a Mouse Model of TNBC Brain Metastases

### Materials and Methods

TNBC brain metastases were generated in mice by intracardiac injection of luciferase-expressing 231-BR cells. Mice were treated with tail vein injection of control (PBS) or h3E10 di-scFv (20 mg/kg) 3×/week for 4 weeks, and brain metastases were tracked by IVIS.

h3E10 di-scFv in this example and the associate
figures refers to SEQ ID NO: 70-Variant 13
DIQMTQSPSSLSASLGDRATITCRASKTVSTSSYSYMHWYQQKPGQPPK
LLIKYASYLESGVPSRFSGSGSGTDFTLTISSLQPEDAATYYCQHSREF
PWTFGGGTKVEIKRADAAPGGGGSGGGGSGGGGSEVQLVESGGGLVQPG
GSLRLSCAASGFTFSNYGMHWVRQAPGKGLEWVSYISSGSSTIYYADSV
KGRFTISRDNAKNSLYLQMNSLRAEDTAVYYCARRGLLLDYWGQGTTVT
VSSASTKGPSVFPLAPLESSGSDIQMTQSPSSLSASLGDRATITCRASK
TVSTSSYSYMHWYQQKPGQPPKLLIKYASYLESGVPSRFSGSGSGTDFT
LTISSLQPEDAATYYCQHSREFPWTFGGGTKVEIKRADAAPGGGGSGGG
GSGGGGSEVQLVESGGGLVQPGGSLRLSCAASGFTFSNYGMHWVRQAPG
KGLEWVSYISSGSSTIYYADSVKGRFTISRDNAKNSLYLQMNSLRAEDT
AVYYCARRGLLLDYWGQGTTVTVSS

### Results

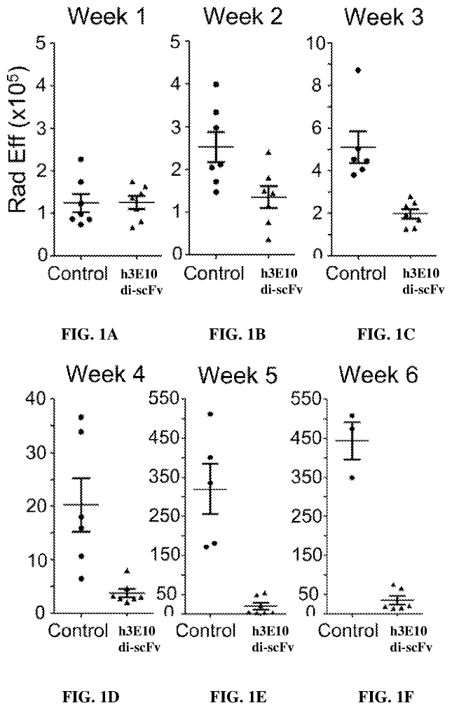

Absolute radiance efficiencies at week 5 are shown in FIGS. 1A-1F.

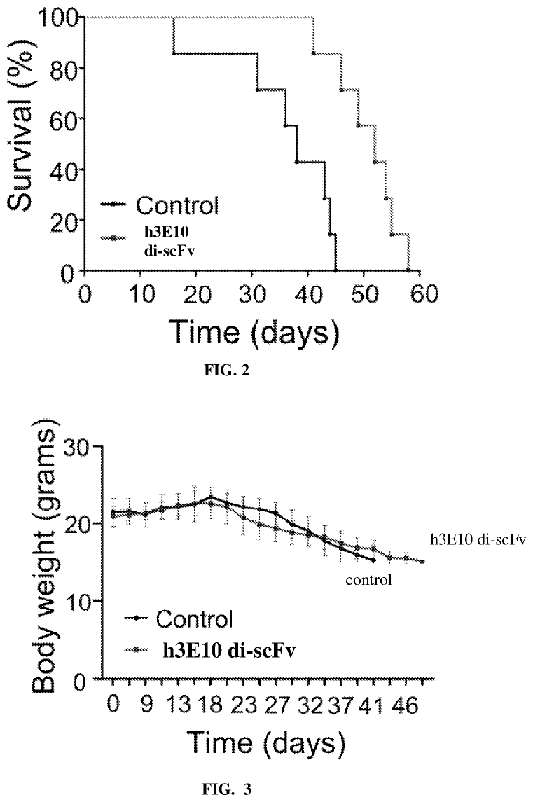

h3E10 di-scFv prolonged survival (median survival 38 and 52 days, P<0.02) without apparent toxicity (FIGS. 2 and 3, respectively).

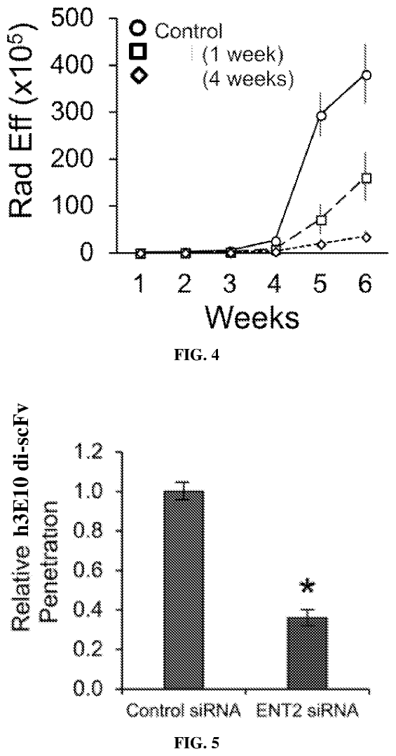

FIG. 4 illustrates differences in dosing over 1 week vs. 4 weeks.

### Example 2: h3E10 Di-scFv Crosses the Blood-Brain Barrier in an ENT2-Dependent Manner

### Materials and Methods

siRNA Knockdown

hCMEC/D3 brain endothelial cells were grown to 50% confluence. 60 nM ENT2 siRNA pool (siGENOME SMARTpool) or non-targeting control siRNA (Dharmacon) were transfected into hCMEC/D3 cells using Lipofectamine RNAiMAX reagent (Invitrogen) according to the manufacturer's instructions. The transfection was repeated the next day to ensure maximum knock down. RNAs were made from cells 3 days after the first transfection and then analyzed by RT-QPCR.

Measurement of mRNA

RNA from hCMEC/D3 cells transfected with ENT2 siRNA or control siRNA were extracted using RNeasy kit (QiaGen) according to the manufacturer's instruction. RNA was reverse transcribed into cDNA using QuantiTect Reverse Transcription Kit (QiaGen) according to the manufacturer protocol using 0.5 μg RNA in a total of 20 μl reaction. The mRNA level of ENT2 and β-actin was assessed using the TaqMan Gene Expression real-time PCR assays (TaqMan probe #Hs01546959_g1 and Hs0160665g1, respectively, Applied Biosystems, Carlsbad, CA, USA). The results were expressed as the threshold cycle (Ct). The relative quantification of the target transcripts normalized to the endogenous control β-actin was determined by the comparative Ct method (ΔCt) and the 2-ΔΔCt method was used to analyze the relative changes in gene expression between the tested samples according to the manufacturer's protocol (User Bulletin No. 2, Applied Biosystems).

Transwell Assay

Immortalized hCMEC/D3 human BECs that recapitulate intercellular junctions to restrict paracellular transport at the BBB are commonly used in Transwell filter assays of drug BBB permeability. hCMEC/D3 cells are seeded onto the apical side of collagencoated Transwell filters, and normal human astrocytes (NHA) are seeded onto the basolateral surface. Filters are transferred to culture plates, which establishes apical and basolateral chambers separated by the BBB model. Efficiency of BBB penetration is tested by adding compounds of interest, in this case h3E10 di-scFv, to the apical chamber, and measuring appearance of compound in the basolateral chamber (Poller, et al., J Neurochem, 107 (5): 1358-1368 (2008) PubMed PMID: 19013850).

h3E10 di-scFv in this example and the associate
figures refers to SEQ ID NO: 70-Variant 13
DIQMTQSPSSLSASLGDRATITCRASKTVSTSSYSYMHWYQQKPGQPPK
LLIKYASYLESGVPSRFSGSGSGTDFTLTISSLQPEDAATYYCQHSREF
PWTFGGGTKVEIKRADAAPGGGGSGGGGSGGGGSEVQLVESGGGLVQPG
GSLRLSCAASGFTFSNYGMHWVRQAPGKGLEWVSYISSGSSTIYYADSV
KGRFTISRDNAKNSLYLQMNSLRAEDTAVYYCARRGLLLDYWGQGTTVT
VSSASTKGPSVFPLAPLESSGSDIQMTQSPSSLSASLGDRATITCRASK
TVSTSSYSYMHWYQQKPGQPPKLLIKYASYLESGVPSRFSGSGSGTDFT
LTISSLQPEDAATYYCQHSREFPWTFGGGTKVEIKRADAAPGGGGSGGG
GSGGGGSEVQLVESGGGLVQPGGSLRLSCAASGFTFSNYGMHWVRQAPG
KGLEWVSYISSGSSTIYYADSVKGRFTISRDNAKNSLYLQMNSLRAEDT
AVYYCARRGLLLDYWGQGTTVTVSS

### Results

h3E10 di-scFv crosses the BBB to suppress the growth of brain tumors (see, e.g., Example 1) Immunofluorescence analysis revealed that ENT2 is expressed in human brain endothelial cells (BECs).

Assays were designed to determine if cell penetration into cells is dependent on ENT2. hCMEC/D3 brain endothelial cells transfected with control siRNA or ENT2 siRNA were treated with h3E10 di-scFv, and then fixed and stained for h3E10 di-scFv using a protein L method. Results presented in FIG. 5 show that ENT2 knockdown impairs h3E10 di-scFv penetration into hCMEC/D3 cells.

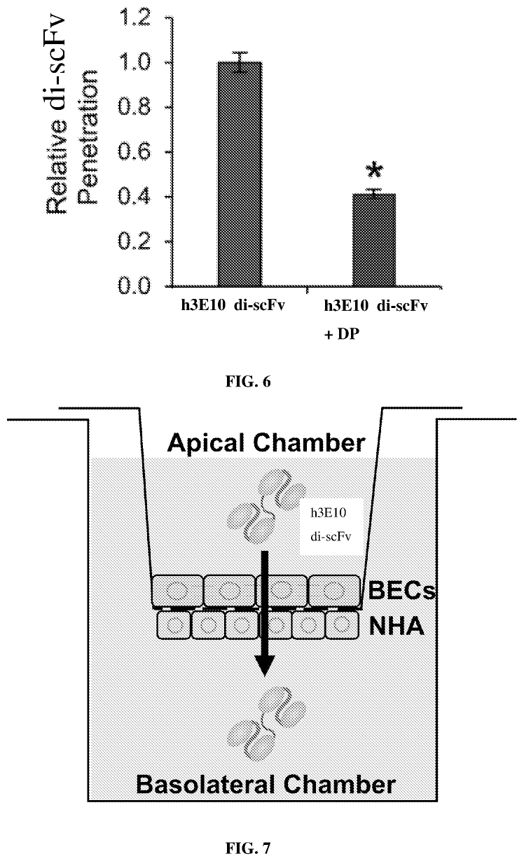

FIG. 6 shows that treatment of the cells with dipyridamole (DP) inhibits the penetration of h3E10 di-scFv into hCMEC/D3 BECs. The observed reduction in h3E10 di-scFv staining in cells treated with DP demonstrates that DP inhibits penetration of h3E10 di-scFv into the BECs.

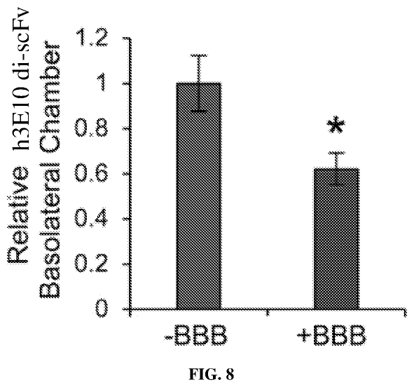

A Transwell filter model (FIG. 7) was used to test the ability of h3E10 di-scFv to cross the human BBB. FIG. 8 shows that h3E10 di-scFv crosses the Transwell filter model of the BBB. The efficiency of h3E10 di-scFv transport across control blank Transwell filters (−BBB) and Transwell filters with BBB (+BBB) was compared by anti-h3E10 di-scFv western blot analysis of basolateral chambers one hour after addition of h3E10 di-scFv to apical chambers. Band intensity was quantified by ImageJ. Quantification of h3E10 di-scFv content in basolateral chambers (relative to control blank filters treated with h3E10 di-scFv) (FIG. 8) is shown, and demonstrates that h3E10 di-scFv crosses the hCMEC/D3 Transwell filter BBB model. Integrity of the model was also confirmed by TEER and exclusion of BSA.

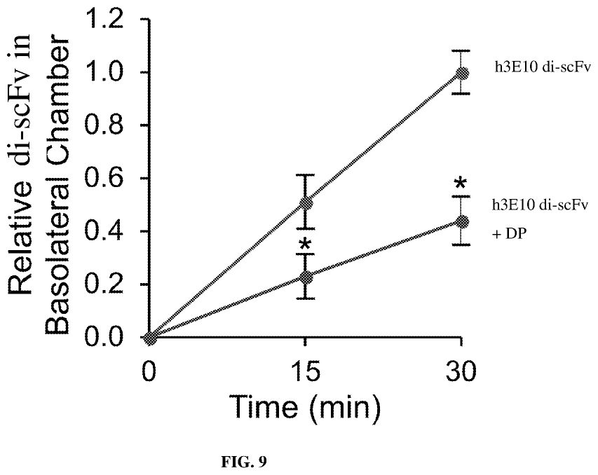

The efficiency of h3E10 di-scFv transport across Transwell filters with BBB in the presence or absence of 50 μM DP was compared by anti-h3E10 di-scFv western and immunoblot analysis of basolateral chambers 15 and 30 minutes after addition of h3E10 di-scFv to apical chambers. Western and immunoblots were quantified by ImageJ. Quantification of h3E10 di-scFv content in basolateral chambers at each time point, relative to the 30 minute time point in the absence of DP, are plotted, and demonstrate that DP inhibits h3E10 di-scFv transport across the hCMEC/D3 Transwell filter BBB model. See FIG. 9. The results indicated that DP inhibits the transport of h3E10 di-scFv across the hCMEC/D3 BBB.

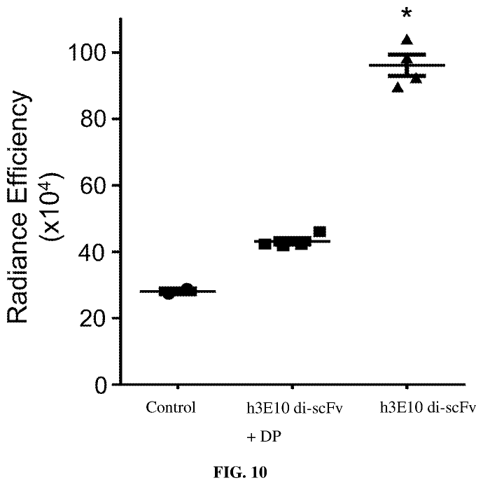

DP inhibits uptake of h3E10 di-scFv into brain tumors. PS30 human GBM glioma stem-like cells (GSCs) were inoculated into the brains of immunodeficient mice to generate orthotopic PDX GBM tumors. These tumors express luciferase, which facilitates tumor visualization by IVIS. h3E10 di-scFv was labeled with IR750 to similarly facilitate its detection by IVIS. Tumor formation was confirmed by IVIS to detect luciferase signal, and mice were then treated with tail vein and intraperitoneal injection of control buffer (N=2), tail vein injection of h3E10 di-scFvIR750 and intraperitoneal injection of control buffer (N=4), or tail vein injection of h3E10 di-scFvIR750 and intraperitoneal injection of DP (70 mg/kg) (N=4). Twenty-four hours after treatment, IVIS measurements to detect h3E10 di-scFv content by IR750 signal demonstrated that DP suppressed uptake of h3E10 di-scFv into the brain tumors. Quantification of radiance efficiencies are shown in FIG. 10.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A method of treating an autoimmune disease comprising administering to a subject in need thereof an effective amount of an inhibitor of a nucleoside transporter to reduce transcellular transport of one or more cell penetrating antibodies into a tissue of the subject.

2. The method of claim 1, wherein the tissue is the brain.

3. The method of claims 1 and 2, wherein the inhibitor reduces transport of the antibody or antibodies across the blood-brain barrier (BBB).

4. The method of any one of claims 1-3, wherein the subject has central nervous system lupus.

5. The method of claim 1, wherein the tissue is the kidney.

6. The method of any one of claims 1-5, wherein the autoimmune disease has one or more symptoms or pathology dependent on or otherwise caused by one or more cell penetrating antibodies.

7. The method of claim 6, wherein one or more of cell penetrating antibodies are nuclear penetrating antibodies.

8. The method of claim 6 or 7, wherein one or more of the antibodies bind to nucleic acids, nucleotides, nucleotides, or a combination thereof.

9. The method of any one of claims 1-8 wherein one or more of the antibodies are internalized and/or transcellularly transported at least in part by the nucleotide transporter.

10. The method of any one of claims 1-9, wherein the autoimmune disease is one or more of systemic lupus erythematosus (lupus or SLE), CNS, lupus, systemic sclerosis (scleroderma), Graves' disease, myasthenia gravis, autoimmune hemolytic anemia, and pemphigus vulgaris, and additionally may contribute to the severity of disease in other autoimmune diseases such as rheumatoid arthritis, autoimmune thrombocytopenia, autoimmune atrophic gastritis of pernicious anemia, myasthenia gravis, Goodpasture's syndrome, diabetes mellitus, multiple sclerosis, Hashimoto's thyroiditis, Crohn's disease, and Sjögren's syndrome, primary biliary cirrhosis, primary sclerosing cholangitis, psoriasis, psoriatic arthritis, POEMS syndrome, dermatomyositis, inclusion body myositis, inflammatory myopathies, vasculitis syndromes including but not limited to Churg-Strauss Syndrome, Wegener granulomatosis, Behcet's disease, Buerger's disease, Kawasaki disease, Takayasu's arteritis, Henoch-Schonlein purpura, Giant cell arteritis, or polyarteritis nodosa.

11. The method of any one of claims 1 to 10, wherein the autoimmune disease is a lupus.

12. The method of claim 11, wherein the lupus includes or is selected from group consisting of CNS, lupus, systemic lupus erythematosus, discoid lupus erythematosus, subacute cutaneous lupus erythematosus, neonatal lupus, and drug-induced lupus.

13. The method of claim 12, wherein the lupus includes CNS lupus.

14. The method of any one of claims 1 to 13, wherein the autoimmune disease is scleroderma.

15. A method of treating lupus comprising administering to a subject in need thereof effective amount of an inhibitor of a nucleoside transporter to reduce one or more symptoms of the lupus.

16. The method of claim 15, wherein the lupus is selected from group consisting of systemic lupus erythematosus, discoid lupus erythematosus, subacute cutaneous lupus erythematosus, neonatal lupus, and drug-induced lupus.

17. A method of treating CNS lupus comprising administering to a subject in need thereof effective amount of an inhibitor of a nucleoside transporter to reduce one or more symptoms of the CNS lupus.

18. A method of treating an autoimmune disease comprising administering to a subject in need thereof an effective amount of an inhibitor of a nucleoside transporter to reduce transduction of one or more cell penetrating antibodies into cells of the subject,
wherein the inhibitor of the nucleoside transporter is dipyridamole or a tautomer, geometrical isomer, optically active form, enantiomeric mixture thereof, pharmaceutically acceptable salt or pharmaceutically active analogue thereof.

19. The method of claim 18, wherein the cells are BBB cells.

20. The method of claim 19, wherein the BBB cells are human endothelial cells.

21. The method of any one of claims 18-20, wherein the tissue is brain tissue.

22. A method of reducing transcellular transport an antibody into or through a tissue in a subject comprising administering the subject an effective amount of an inhibitor of a nucleoside transporter to reduce transcellular transport of the antibody into or through the tissue,
wherein the subject is administered the antibody separately or together with the inhibitor of the nucleoside transporter,
optionally wherein the subject has cancer.

23. A method of reducing translocation of an antibody into cells of a subject comprising administering to the subject an effective amount of an inhibitor of a nucleoside transporter to reduce translocation of the antibody into the cells,
wherein the subject is administered the antibody separately or together with the inhibitor of the nucleoside transporter,
optionally wherein the subject has cancer.

24. The method of claim 22 or 23, wherein the subject is administered the antibody in an effective amount to inhibit DNA repair or directly damage DNA separately or together with the inhibitor of the nucleoside transporter, optionally wherein the antibody is therapeutic for the cancer.

25. The method of any one of claims 22-24, wherein the antibody is selected from the group consisting of 3E10, 5C6, fragments and fusions of 3E10 and 5C6, and variants and humanized forms of 3E10, 5C6, and fragments and fusions of 3E10 and 5C6.

26. The method of claim 25, wherein the antibody is a humanized 3E10 di-scFv.

27. The method of claim 26, wherein the humanized 3E10 di-scFv is SEQ ID NO:70, or variant thereof with 90% sequence identity thereto.

28. The method of any one of claims 1-27, wherein the inhibitor of the nucleoside transporter inhibits activity or expression of one or more of ENT1, ENT2, ENT3, or ENT4, optionally at least or only ENT2.

29. The method of any one of claims 1-28, wherein the inhibitor of the nucleoside transporter is a purine nucleoside analogue, a pyrimidopyrimidine or pteridine derivative, or a flazine calcium channel blocker.

30. The method of any one of claim 1-29, wherein the inhibitor of the nucleoside transporter is dipyridamole or a tautomer, geometrical isomer, optically active form, enantiomeric mixture thereof, pharmaceutically acceptable salt or pharmaceutically active analogue thereof.

31. The method of claim 30, wherein the inhibitor of the nucleoside transporter is dipyridamole or pharmaceutically acceptable salt thereof.

32. The method of claim 30, wherein the inhibitor of the nucleoside transporter is a dipyridamole analogue or pharmaceutically acceptable salt thereof.

33. The method of claim 32, wherein the dipyridamole analogue is a compound of formula I.

34. The method of claim 32, wherein the dipyridamole analogue is a compound of any one of Tables 1-5.

35. The method of any one of claims 1-28, wherein the inhibitor of the nucleoside transporter is a peptide.

36. The method of any of claims 1-28, wherein the inhibitor of the nucleoside transporter is a binding protein.

37. The method of claim 36, wherein the inhibitor of the nucleoside transporter is an antibody or fragment thereof.

38. The method of any one of claims 1-28, wherein the inhibitor of the nucleoside transporter is an oligonucleotide inhibitor.

39. The method of claim 38, wherein the oligonucleotide inhibitor is an antisense RNA or DNA, siRNA or siDNA, miRNA, miRNA mimic, shRNA or DNA and Chimeric Antisense DNA or RNA.

40. The method claim 39, wherein the inhibitor of the nucleoside transporter is an siRNA, shRNA, or miRNA.

41. The method of any one of claims 1-40 wherein the inhibitor is administered to a subject in need thereof by a parenteral, enteral, transdermal, or transmucosal route of administration.

42. A composition comprising an effective amount an inhibitor of any one of claims 1-41 to reduce one or more symptoms of an autoimmune disease in a subject in need thereof.

43. The composition of claim 42, wherein the autoimmune disease is or includes CNS lupus.
